(19) United States
(12) Patent Application Publication
(10) Pub. No.: US 2023/0174454 A1
(43) Pub. Date: Jun. 8, 2023

(54) PROCESS OF SYNTHESIZING AND PURIFYING (3R)-HYDROXYBUTYL (3R)-HYDROXYBUTANOATE

(71) Applicant: Genomatica, Inc., San Diego, CA (US)

(72) Inventors: Bo CHEN, San Diego, CA (US); Iman SAFARI, San Diego, CA (US); Seth LEVINE, Encinitas, CA (US)

(21) Appl. No.: 17/998,111

(22) PCT Filed: May 10, 2021
(86) PCT No.: PCT/US2021/031531

§ 371 Date: Nov. 7, 2022

Related U.S. Application Data
(60) Provisional application No. 63/023,776, filed on May 12, 2020.

(51) Int. Cl. C07C 67/08; C07C 67/54; C12P 7/18

(52) U.S. Cl. CPC C07C 67/08; C07C 67/54; C12P 7/18

(57) ABSTRACT

A process for synthesizing (R)-3 -hy droxybutyl (R)-3 -hy droxybutanoate from ethyl (R)-3-hydroxybutanoate and (R)-1,3-butanediol, as well a process for synthesizing (R)-3-hy droxybutyl (R)-3-hy droxybutanoate from (R)-3-hydroxybutyric acid and (R)-1,3-butanediol. Also provided are processes for isolating (R)-3-hy droxybutyl (R)-3-hy droxybutanoate, including from a fermentation broth.

### CROSS REFERENCE TO RELATED APPLICATIONS

This Application claims the benefit of U.S. Provisional Application No. 63/023,776, filed May 12, 2020, the disclosure of which is incorporated herein by reference in its entirety.

### BACKGROUND

Certain compounds containing a β-hydroxybutyrate moiety have pharmaceutical and nutritional properties. One example is (3R)-hydroxybutyl (3R)-hydroxybutyrate, which has been studied as a source of nutritional ketones. Regulatory Toxicology and Pharmacology 63(2012), 196-208.

Thus, there is a need to develop a process for the synthesis and purification of (R)-3-hydroxybutyl (R)-3-hydroxybutanoate.

### SUMMARY

In some aspects, embodiments disclosed herein relate to processes of isolating (3R)-hydroxybutyl (3R)-hydroxybutyrate from a fermentation broth that includes separating a liquid fraction enriched in (3R)-hydroxybutyl (3R)-hydroxybutyrate from a solid fraction including cells, removing salts from said liquid fraction, removing water from said liquid fraction, and purifying (R)-3-hydroxybutyl (R)-3-hydroxybutanoate.

In some aspects, embodiments disclosed herein relate to processes of isolating (3R)-hydroxybutyl (3R)-hydroxybutyrate from a fermentation broth that includes separating a liquid fraction enriched in (3R)-hydroxybutyl (3R)-hydroxybutyrate from a solid fraction including cells, and purifying (3R)-hydroxybutyl (3R)-hydroxybutyrate by liquid-liquid extraction.

In some aspects, embodiments disclosed herein relate to synthesizing (3R)-hydroxybutyl (3R)-hydroxybutyrate from (R)-3-hydroxybutanoic acid.

In some aspects, embodiments disclosed herein relate to synthesizing (3R)-hydroxybutyl (3R)-hydroxybutyrate from ethyl (R)-3-hydroxybutanoate.

### BRIEF DESCRIPTION OF THE DRAWINGS

FIG. 1 shows a schematic of the overall process of the formation of (3R)-hydroxybutyl (3R)-hydroxybutyrate starting with the fermentation of (R)-1,3-butanediol and (R)-3-hydroxybutyric acid.

FIG. 2 shows a schematic of an integrated process of esterifications and downstream separation units.

FIG. 3 shows a schematic of the chemical reaction of (R)-3-hydroxybutyric acid to (R)-ethyl-3-hydroxybutyrate in a reactive distillation column.

FIG. 4 shows a schematic of the overall process for production and purification of (3R)-hydroxybutyl (3R)-hydroxybutyrate from (R)-1,3-butanediol and (R)-3-hydroxybutyric acid.

FIG. 5 shows a schematic of the process of the formation of (3R)-hydroxybutyl (3R)-hydroxybutyrate from an enzymatic reaction with (R)-1,3-butanediol to (3R)-hydroxybutyl (3R)-hydroxybutyrate.

FIG. 6 shows a schematic of the process of the formation of (R)-ethyl-3-hydroxybutyrate by fermentation and reactive distillation with (R)-1,3-butanediol to form (3R)-hydroxybutyl (3R)-hydroxybutyrate.

FIG. 7 shows the overall process for production and purification of (3R)-hydroxybutyl (3R)-hydroxybutyrate through direct fermentation.

FIG. 8 shows a schematic of distillation units.

FIG. 9 shows the liquid-liquid extraction process for recovery of (3R)-hydroxybutyl (3R)-hydroxybutyrate from the fermentation broth when a lower boiling point solvent is used.

FIG. 10 shows the liquid-liquid extraction process for recovery of (3R)-hydroxybutyl (3R)-hydroxybutyrate from the fermentation broth when a higher boiling point solvent is used.

### DETAILED DESCRIPTION

The compound (R)-3-hydroxybutyl (R)-3-hydroxybutanoate (“Ketone Ester”) has the structure:

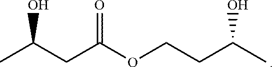

Ketone Ester can be made by the processes disclosed in US 2016/0108442, which is incorporated in its entirety.

One embodiment provided herein is a process for preparing the compound of Formula

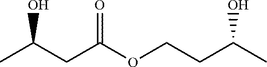

the process including the steps of performing a first esterification between HOR and a compound of Formula (II):

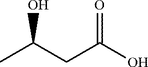

to form the compound of Formula (III) in a first esterification product stream:

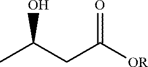

followed by performing a second esterification with the compound of Formula (IV):

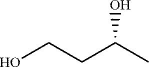

to produce the compound of Formula (I) and HOR in a second esterification product stream, wherein R is methyl, ethyl, propyl, or isopropyl.

In one embodiment, the first esterification is promoted with an acid.

In one embodiment, the acid is selected from sulfuric acid, hydrochloric acid, acetic acid, benzoic acid, tosylic acid, candium(III) triflate, trifluoroacetic acid, phosphoric acid nitric acid, sulfamic acid, sulfonic acids, formic acid, acetic acid, lactic acid, propionic acid, oxalic acid, malonic acid, succinic acid, glutaric acid, or adipic acid.

In one embodiment, the second esterification is promoted with an acid.

In one embodiment, the acid is selected from sulfuric acid, hydrochloric acid, acetic acid, benzoic acid, tosylic acid, candium(III) triflate, trifluoroacetic acid, phosphoric acid nitric acid, sulfamic acid, sulfonic acids, formic acid, acetic acid, lactic acid, propionic acid, oxalic acid, malonic acid, succinic acid, glutaric acid, or adipic acid.

In one embodiment, the first esterification is promoted with an immobilized enzyme.

In one embodiment, the immobilized enzyme is an lipase.

In one embodiment, the lipase is selected from Novozyme 435, patatin, or Candida.

In one embodiment, the immobilized enzyme is an esterase. In one embodiment the esterase is a carboxylesterase.

In one embodiment, the second esterification is promoted with an immobilized enzyme. In one embodiment, the immobilized enzyme is a lipase.

In one embodiment, the lipase is selected from Novozyme 435, patatin, or Candida.

In one embodiment, the immobilized enzyme is an esterase. In one embodiment the esterase is a carboxylesterase.

In one embodiment, the HOR generated during the second esterification is recovered and recycled.

In one embodiment, the HOR generated during the second esterification is aqueous.

In one embodiment provided herein, a process for preparing (R)-3-hydroxybutyl (R)-3-hydroxybutanoate, including the steps of performing a first esterification between HOR and a compound of Formula (I):

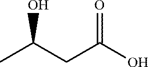

to form the compound of Formula (II) and water in a first esterification product stream:

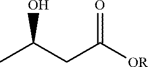

subjecting the first esterification product stream to distillation to remove water to form a concentrated first esterification product stream;
subjecting the concentrated first esterification product stream to distillation to form an enriched first esterification product stream and a heavies stream including the compound of Formula (I);
subjecting the enriched first esterification product stream to a second esterification with the compound of Formula (III):

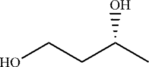

to produce (R)-3-hydroxybutyl (R)-3-hydroxybutanoate and HOR in a second esterification product stream, wherein R is methyl, ethyl, propyl, or isopropyl.

In one embodiment, the heavies stream including the compound of Formula (I) is recycled into the first esterification.

In one embodiment, the process further includes purifying the second esterification product stream.

In one embodiment, water is removed during the esterification reaction.

In one embodiment, water removal during the esterification reaction is accomplished with reactive distillation.

In one embodiment, purifying the second esterification product stream is accomplished by distillation.

In one embodiment, distillation includes:
(a) subjecting the second esterification product stream to a first column distillation procedure to remove materials with a boiling point lower than (R)-3-hydroxybutyl (R)-3-hydroxybutanoate from the second esterification product stream to produce a first (R)-3-hydroxybutyl (R)-3-hydroxybutanoate-containing product stream; and
(b) subjecting the first (R)-3-hydroxybutyl (R)-3-hydroxybutanoate-containing product stream to a second column distillation procedure to remove materials with boiling points higher than (R)-3-hydroxybutyl (R)-3-hydroxybutanoate as a first high-boilers stream, to produce a purified (R)-3-hydroxybutyl (R)-3-hydroxybutanoate product.

In one embodiment, the process further includes:
(c) subjecting the first high-boilers stream to wiped-film evaporation (WFE) to produce a first WFE distillate and subjecting the first WFE distillate to step (b);
(d) subjecting the first (R)-3-hydroxybutyl (R)-3-hydroxybutanoate-containing product stream, prior to performing step (b), to an intermediate column distillation procedure to remove materials with boiling points higher than (R)-3-hydroxybutyl (R)-3-hydroxybutanoate as a second high-boilers stream; and
(e) subjecting the second high-boilers stream to wiped-film evaporation (WFE) producing a second WFE distillate and subjecting the second WFE distillate to step (d).

In one embodiment, the HOR generated during the second esterification is recovered and recycled.

In one embodiment, the HOR generated during the second esterification is aqueous.

In one embodiment, the first column distillation procedure and second column distillation procedures are each performed at pressures equal to or less than atmospheric pressure.

In one embodiment, the pressure of the first column distillation procedure differs from the pressure of the second distillation procedure.

In one embodiment, the process further includes subjecting the purified (R)-3-hydroxybutyl (R)-3-hydroxybutanoate product to a polishing column.

In one embodiment, the polishing column is an ion exchange column.

In one embodiment, the ion exchange column uses an exchange resin that is an anion exchange resin.

In one embodiment, the ion exchange column uses an exchange resin that is a cation exchange resin.

In one embodiment, the purified (R)-3-hydroxybutyl (R)-3-hydroxybutanoate is at least 90% pure.

In one embodiment, the first esterification is promoted with an acid.

In one embodiment, the acid is selected from sulfuric acid, hydrochloric acid, acetic acid, benzoic acid, tosylic acid, candium(III) triflate, trifluoroacetic acid, phosphoric acid nitric acid, sulfamic acid, sulfonic acids, formic acid, acetic acid, lactic acid, propionic acid, oxalic acid, malonic acid, succinic acid, glutaric acid, or adipic acid.

In one embodiment, the second esterification is promoted with an acid.

In one embodiment, the acid is selected from sulfuric acid, hydrochloric acid, acetic acid, benzoic acid, tosylic acid, candium(III) triflate, trifluoroacetic acid, phosphoric acid nitric acid, sulfamic acid, sulfonic acids, formic acid, acetic acid, lactic acid, propionic acid, oxalic acid, malonic acid, succinic acid, glutaric acid, or adipic acid.

In one embodiment, the first esterification is promoted with an immobilized enzyme.

In one embodiment, the immobilized enzyme is an lipase.

In one embodiment, the lipase is selected from Novozyme 435, patatin, or Candida.

In one embodiment, the immobilized enzyme is an esterase. In one embodiment the esterase is a carboxylesterase.

In one embodiment, the second esterification is promoted with an immobilized enzyme.

In one embodiment, the immobilized enzyme is a lipase.

In one embodiment, the lipase is selected from Novozyme 435, patatin, or Candida.

In one embodiment, the immobilized enzyme is an esterase. In one embodiment the esterase is a carboxylesterase.

In one embodiment provided herein is a process for preparing (R)-3-hydroxybutyl (R)-3-hydroxybutanoate, including the step of esterifying ethyl (R)-3-hydroxybutanoate with (R)-1,3-butanediol in a reactor to form a product stream including (R)-3-hydroxybutyl (R)-3-hydroxybutanoate and ethanol.

In one embodiment, the esterification is promoted with an acid.

In one embodiment, the acid is selected from sulfuric acid, hydrochloric acid, acetic acid, benzoic acid, tosylic acid, candium(III) triflate, trifluoroacetic acid, phosphoric acid nitric acid, sulfamic acid, sulfonic acids, formic acid, acetic acid, lactic acid, propionic acid, oxalic acid, malonic acid, succinic acid, glutaric acid, or adipic acid.

In one embodiment, the second esterification is promoted with an immobilized enzyme.

In one embodiment, the immobilized enzyme is a lipase.

In one embodiment, the lipase is selected from Novozyme 435, patatin, or Candida.

In one embodiment, the immobilized enzyme is an esterase. In one embodiment the esterase is a carboxylesterase.

In one embodiment, the ethanol generated during the second esterification is recovered and recycled.

Wherein the ethanol generated during the second esterification is aqueous.

In one embodiment, the reaction operates at a temperature of 0° C. to 120° C.

In one embodiment, the reactor operates at a temperature of 10° C. to 50° C.

In one embodiment, the reactor operates under reduced pressure. In one embodiment, the pressure is between 5 and 400 mmHg.

In one embodiment, the reactor operates under positive pressure. In one embodiment, the pressure is between 1 and 2 atmospheres.

In one embodiment, the product stream is subjected to distillation.

In one embodiment, the distillation includes:
(a) subjecting the product stream to a first column distillation procedure to remove materials with a boiling point lower than (R)-3-hydroxybutyl (R)-3-hydroxybutanoate from the product stream to produce a first (R)-3-hydroxybutyl (R)-3-hydroxybutanoate-containing product stream; and
(b) subjecting the first (R)-3-hydroxybutyl (R)-3-hydroxybutanoate-containing product stream to a second column distillation procedure to remove materials with boiling points higher than (R)-3-hydroxybutyl (R)-3-hydroxybutanoate as a first high-boilers stream, to produce a purified (R)-3-hydroxybutyl (R)-3-hydroxybutanoate product.

In one embodiment, the process further includes:
(c) subjecting the first high-boilers stream to wiped-film evaporation (WFE) to produce a first WFE distillate and subjecting the first WFE distillate to step (a);
(d) subjecting the first (R)-3-hydroxybutyl (R)-3-hydroxybutanoate-containing product stream, prior to performing step (b), to an intermediate column distillation procedure to remove materials with boiling points higher than (R)-3-hydroxybutyl (R)-3-hydroxybutanoate as a second high-boilers stream; and
(e) subjecting the second high-boilers stream to wiped-film evaporation (WFE) producing a second WFE distillate and subjecting the second WFE distillate to step (d).

In one embodiment, the purified (R)-3-hydroxybutyl (R)-3-hydroxybutanoate is at least 90% pure.

In one embodiment, the first column distillation procedure and second column distillation procedures are each performed at pressures equal to or less than atmospheric pressure.

In one embodiment, the pressure of the first column distillation procedure differs from the pressure of the second distillation procedure.

In one embodiment, the process further includes subjecting the purified (R)-3-hydroxybutyl (R)-3-hydroxybutanoate product to a polishing column.

In one embodiment, the polishing column is an ion exchange column.

In one embodiment, the ion exchange column uses an exchange resin that is an anion exchange resin.

In one embodiment, the ion exchange column uses an exchange resin that is a cation exchange resin.

In one embodiment, the materials with a boiling point lower than (R)-3-hydroxybutyl (R)-3-hydroxybutanoate include (R)-3-hydroxybutanoate and (R)-1,3-butanediol.

In one embodiment, the (R)-3-hydroxybutanoate and (R)-1,3-butanediol are recycled back into the reactor.

In one embodiment, ethanol is removed during the esterification reaction.

In one embodiment, ethanol removal during the esterification reaction is accomplished with reactive distillation.

One embodiment provided herein is a process for preparing (R)-3-hydroxybutyl (R)-3-hydroxybutanoate, including the step of esterifying (R)-3-hydroxybutanoic acid with (R)-1,3-butanediol in a reactor to form a product stream including (R)-3-hydroxybutyl (R)-3-hydroxybutanoate and ethanol.

In one embodiment, the esterification is promoted with an acid.

In one embodiment, the acid is selected from sulfuric acid, hydrochloric acid, acetic acid, benzoic acid, tosylic acid, candium(III) triflate, trifluoroacetic acid, phosphoric acid nitric acid, sulfamic acid, sulfonic acids, formic acid, acetic acid, lactic acid, propionic acid, oxalic acid, malonic acid, succinic acid, glutaric acid, or adipic acid.

In one embodiment, the second esterification is promoted with an immobilized enzyme.

In one embodiment, the immobilized enzyme is a lipase.

In one embodiment, the lipase is selected from Novozyme 435, patatin, or Candida.

In one embodiment, the immobilized enzyme is an esterase. In one embodiment the esterase is a carboxylesterase.

In one embodiment, the reactor operates at a temperature of 0° C. to 120° C.

In one embodiment, the reactor operates at a temperature of 10° C. to 50° C.

In one embodiment, the reactor operates under reduced pressure. In one embodiment, the pressure is between 5 and 400 mmHg.

In one embodiment, the reactor operates under positive pressure. In one embodiment, the pressure is between 1 and 2 atmospheres.

In one embodiment, the product stream is subjected to distillation.

In one embodiment, the distillation includes:
(a) subjecting the product stream to a first column distillation procedure to remove materials with a boiling point lower than (R)-3-hydroxybutyl (R)-3-hydroxybutanoate from the product stream to produce a first (R)-3-hydroxybutyl (R)-3-hydroxybutanoate-containing product stream; and
(b) subjecting the first (R)-3-hydroxybutyl (R)-3-hydroxybutanoate-containing product stream to a second column distillation procedure to remove materials with boiling points higher than (R)-3-hydroxybutyl (R)-3-hydroxybutanoate as a first high-boilers stream, to produce a purified (R)-3-hydroxybutyl (R)-3-hydroxybutanoate product.

In one embodiment, the process further includes:
(c) subjecting the first high-boilers stream to wiped-film evaporation (WFE) to produce a first WFE distillate and subjecting the first WFE distillate to step (a);
(d) subjecting the first (R)-3-hydroxybutyl (R)-3-hydroxybutanoate-containing product stream, prior to performing step (b), to an intermediate column distillation procedure to remove materials with boiling points higher than (R)-3-hydroxybutyl (R)-3-hydroxybutanoate as a second high-boilers stream; and
(e) subjecting the second high-boilers stream to wiped-film evaporation (WFE) producing a second WFE distillate and subjecting the second WFE distillate to step (d).

In one embodiment, the purified (R)-3-hydroxybutyl (R)-3-hydroxybutanoate is at least 90% pure.

In one embodiment, the first column distillation procedure and second column distillation procedures are each performed at pressures equal to or less than atmospheric pressure.

In one embodiment, the pressure of the first column distillation procedure differs from the pressure of the second distillation procedure.

In one embodiment, the process further includes subjecting the purified (R)-3-hydroxybutyl (R)-3-hydroxybutanoate product to a polishing column.

In one embodiment, the polishing column is an ion exchange column.

In one embodiment, the ion exchange column uses an exchange resin that is an anion exchange resin.

In one embodiment, the ion exchange column uses an exchange resin that is a cation exchange resin.

In one embodiment, the materials with a boiling point lower than (R)-3-hydroxybutyl (R)-3-hydroxybutanoate include (R)-3-hydroxybutanoate and (R)-1,3-butanediol.

In one embodiment, the (R)-3-hydroxybutanoate and (R)-1,3-butanediol are recycled back into the reactor.

In one embodiment, water is removed during the esterification reaction.

In one embodiment, water removal during the esterification reaction is accomplished with reactive distillation.

One embodiment provided herein is a process of isolating (R)-3-hydroxybutyl (R)-3-hydroxybutanoate from a fermentation broth, the process including:
separating a liquid fraction enriched in (R)-3-hydroxybutyl (R)-3-hydroxybutanoate from a solid fraction of the fermentation broth including cells;
contacting the cell-free fermentation broth with an extraction solvent in a solvent contact column to make an extraction solvent enriched in (R)-3-hydroxybutyl (R)-3-hydroxybutanoate;
removing the extraction solvent enriched in (R)-3-hydroxybutyl (R)-3-hydroxybutanoate; and
purifying the extraction solvent enriched in (R)-3-hydroxybutyl (R)-3-hydroxybutanoate.

In one embodiment, the separation step includes filtration or centrifugation.

In one embodiment, the centrifugation is accomplished with a disc-stack centrifuge or a decanter centrifuge.

In one embodiment, the filtration consists of ultrafiltration or microfiltration.

In one embodiment, the ultrafiltration includes filtering through a membrane having a pore size from about 0.005 to about 0.1 microns.

In one embodiment, the microfiltration includes filtering through a membrane having a pore size from about 0.1 microns to about 5.0 microns.

In one embodiment, purifying the extraction solvent enriched in (R)-3-hydroxybutyl (R)-3-hydroxybutanoate is accomplished by distillation.

In one embodiment, the distillation includes:
(a) subjecting the extraction solvent enriched in (R)-3-hydroxybutyl (R)-3-hydroxybutanoate to a first column distillation procedure to remove materials with a boiling point lower than (R)-3-hydroxybutyl (R)-3-hydroxybutanoate from the extraction solvent enriched in (R)-3-hydroxybutyl (R)-3-hydroxybutanoate to produce a first (R)-3-hydroxybutyl (R)-3-hydroxybutanoate-containing product stream and a recovered extraction solvent stream; and
(b) subjecting the first (R)-3-hydroxybutyl (R)-3-hydroxybutanoate-containing product stream to a second column distillation procedure to remove materials with boiling points higher than (R)-3-hydroxybutyl (R)-3-hydroxybutanoate as a first high-boilers stream, to produce a purified (R)-3-hydroxybutyl (R)-3-hydroxybutanoate product.

In one embodiment, the process further includes:
(c) subjecting the first high-boilers stream to wiped-film evaporation (WFE) to produce a first WFE distillate and subjecting the first WFE distillate to step (b);
(d) subjecting the first (R)-3-hydroxybutyl (R)-3-hydroxybutanoate-containing product stream, prior to performing step (b), to an intermediate column distillation procedure to remove materials with boiling points higher than (R)-3-hydroxybutyl (R)-3-hydroxybutanoate as a second high-boilers stream; and
(e) subjecting the second high-boilers stream to wiped-film evaporation (WFE) producing a second WFE distillate and subjecting the second WFE distillate to step (d).

In one embodiment, the (R)-3-hydroxybutyl (R)-3-hydroxybutanoate is bioderived.

In one embodiment, the first column distillation procedure and second column distillation procedures are each performed at pressures equal to or less than atmospheric pressure.

In one embodiment, the pressure of the first column distillation procedure differs from the pressure of the second distillation procedure.

In one embodiment, the process further includes subjecting the purified (R)-3-hydroxybutyl (R)-3-hydroxybutanoate product to a polishing column.

In one embodiment, the polishing column is an ion exchange column.

In one embodiment, the ion exchange column uses an exchange resin that is an anion exchange resin.

In one embodiment, the ion exchange column uses an exchange resin that is a cation exchange resin.

In one embodiment, the recovered extraction solvent stream is recycled to the solvent contact column.

In one embodiment, the fermentation broth includes (R)-3-hydroxybutyl (R)-3-hydroxybutanoate at a concentration of about 1%-50% by weight of (R)-3-hydroxybutyl (R)-3-hydroxybutanoate.

In one embodiment, the purified (R)-3-hydroxybutyl (R)-3-hydroxybutanoate is at least 90% pure.

In one embodiment, the solvent contact column is operated at room temperature and atmospheric pressure.

In one embodiment, the extraction solvent is 1-hexanol, 1-butanol, or tributyl phosphate.

In one embodiment, the diameter of the solvent contact column is 1 cm to 10 m.

In one embodiment, the solvent contact column is static.

In one embodiment, the static solvent contact column is a structured packing column, random packing column, or a column including a sieve tray.

In one embodiment, the solvent contact column is agitated.

In one embodiment, the solvent contact column is agitated for a period of time.

In one embodiment, the agitation period is 1 second to 10 hours.

In one embodiment, the agitated solvent contact column is a rotating disc contactor or a pulsed column.

In one embodiment, the agitated solvent contact column is a Karr® column.

In one embodiment, the agitated solvent contact column is a Scheibel® column.

In one embodiment, the solvent contact column is a mixer-settler.

In one embodiment, the fermentation broth includes (R)-3-hydroxybutyl (R)-3-hydroxybutanoate at a concentration of about 5%-15% by weight of (R)-3-hydroxybutyl (R)-3-hydroxybutanoate.

In one embodiment, the purified (R)-3-hydroxybutyl (R)-3-hydroxybutanoate product is greater than 90% (w/w), 91% (w/w), 92% (w/w), 93% (w/w), 94% (w/w), 95% (w/w), 96% (w/w), 97%, (w/w) 98% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w) or 99.9% (w/w), (R)-3-hydroxybutyl (R)-3-hydroxybutanoate.

In one embodiment, the recovery of (R)-3-hydroxybutyl (R)-3-hydroxybutanoate in the purified (R)-3-hydroxybutyl (R)-3-hydroxybutanoate product from the crude (R)-3-hydroxybutyl (R)-3-hydroxybutanoate mixture is greater than 40%, 50%, 60%, 70%, 80%, 90%, 95%, 96%, 97%, 98% or 99%.

In one embodiment, the (R)-3-hydroxybutyl (R)-3-hydroxybutanoate is bioderived.

In one embodiment, the distillation includes:
(a) subjecting the extraction solvent enriched in (R)-3-hydroxybutyl (R)-3-hydroxybutanoate to a first column distillation procedure to remove materials with a boiling point lower than (R)-3-hydroxybutyl (R)-3-hydroxybutanoate from the extraction solvent enriched in (R)-3-hydroxybutyl (R)-3-hydroxybutanoate to produce a first (R)-3-hydroxybutyl (R)-3-hydroxybutanoate-containing product stream; and
(b) subjecting the first (R)-3-hydroxybutyl (R)-3-hydroxybutanoate-containing product stream to a second column distillation procedure to remove materials with boiling points higher than (R)-3-hydroxybutyl (R)-3-hydroxybutanoate as a first high-boilers stream, to produce a purified (R)-3-hydroxybutyl (R)-3-hydroxybutanoate product and a recovered extraction solvent stream.

In one embodiment, the process further includes:
(c) subjecting the first high-boilers stream to wiped-film evaporation (WFE) to produce a first WFE distillate and subjecting the first WFE distillate to step (b);
(d) subjecting the first (R)-3-hydroxybutyl (R)-3-hydroxybutanoate-containing product stream, prior to performing step (b), to an intermediate column distillation procedure to remove materials with boiling points higher than (R)-3-hydroxybutyl (R)-3-hydroxybutanoate as a second high-boilers stream; and
(e) subjecting the second high-boilers stream to wiped-film evaporation (WFE) producing a second WFE distillate and subjecting the second WFE distillate to step (d).

In one embodiment, the first column distillation procedure and second column distillation procedures are each performed at pressures equal to or less than atmospheric pressure.

In one embodiment, the pressure of the first column distillation procedure differs from the pressure of the second distillation procedure.

In one embodiment, the recovered extraction solvent stream is recycled to the solvent contact column.

In one embodiment, the process further includes subjecting the purified (R)-3-hydroxybutyl (R)-3-hydroxybutanoate product to a polishing column.

In one embodiment, the polishing column is an ion exchange column.

In one embodiment, the ion exchange column uses an exchange resin that is an anion exchange resin.

In one embodiment, the ion exchange column uses an exchange resin that is a cation exchange resin.

One embodiment provided herein is a process of isolating (R)-3-hydroxybutyl (R)-3-hydroxybutanoate from a fermentation broth including
separating a liquid fraction enriched in (R)-3-hydroxybutyl (R)-3-hydroxybutanoate from a solid fraction including cells, wherein said step of separating said liquid fraction includes microfiltration or ultrafiltration, and nanofiltration;
removing salts from said liquid fraction, wherein salts are removed by ion exchange;
reducing water from said liquid fraction, wherein removing water is accomplished by evaporation; and
purifying (R)-3-hydroxybutyl (R)-3-hydroxybutanoate from said liquid fraction.

In one embodiment, the microfiltration includes filtering through a membrane having a pore size from about 0.1 microns to about 5.0 microns

In one embodiment, the ultrafiltration includes filtering through a membrane having a pore size from about 0.005 to about 0.1 microns.

In one embodiment, the nanofiltration includes filtering through a membrane having a pore size from about 0.0005 microns to about 0.005 microns

In one embodiment, the evaporation is accomplished with an evaporator system.

In one embodiment, the evaporator system includes an evaporator selected from the group consisting of a falling film evaporator, a short path falling film evaporator, a forced circulation evaporator, a plate evaporator, a circulation evaporator, a fluidized bed evaporator, a rising film evaporator, a counterflow-trickle evaporator, a stirrer evaporator, and a spiral tube evaporator.

In one embodiment, the reduction of water is from about 85% by weight to about 15% by weight.

In one embodiment, the purifying is accomplished by distillation.

In one embodiment, the distillation includes:
(a) subjecting the liquid fraction containing (R)-3-hydroxybutyl (R)-3-hydroxybutanoate to a first column distillation procedure to remove materials with boiling points higher than (R)-3-hydroxybutyl (R)-3-hydroxybutanoate from the liquid fraction containing (R)-3-hydroxybutyl (R)-3-hydroxybutanoate to produce a first (R)-3-hydroxybutyl (R)-3-hydroxybutanoate-containing product stream and a high-boilers stream; and
(b) subjecting the first (R)-3-hydroxybutyl (R)-3-hydroxybutanoate-containing product stream to a second column distillation procedure to remove materials with boiling points lower than (R)-3-hydroxybutyl (R)-3-hydroxybutanoate as a first low-boilers stream, to produce a purified (R)-3-hydroxybutyl (R)-3-hydroxybutanoate product.

In one embodiment, the process further includes:
(c) subjecting the high-boilers stream to wiped-film evaporation (WFE) to produce a first WFE distillate and subjecting the first WFE distillate to step (a).

In one embodiment, the (R)-3-hydroxybutyl (R)-3-hydroxybutanoate is bioderived.

In one embodiment, the first column distillation procedure and second column distillation procedures are each performed at pressures equal to or less than atmospheric pressure.

In one embodiment, the pressure of the first column distillation procedure differs from the pressure of the second distillation procedure.

In one embodiment, the purified (R)-3-hydroxybutyl (R)-3-hydroxybutanoate product is greater than 90% (w/w), 91% (w/w), 92% (w/w), 93% (w/w), 94% (w/w), 95% (w/w), 96% (w/w), 97%, (w/w) 98% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w) or 99.9% (w/w), (R)-3-hydroxybutyl (R)-3-hydroxybutanoate.

In one embodiment, the recovery of (R)-3-hydroxybutyl (R)-3-hydroxybutanoate in the purified (R)-3-hydroxybutyl (R)-3-hydroxybutanoate product from the crude (R)-3-hydroxybutyl (R)-3-hydroxybutanoate mixture is greater than 40%, 50%, 60%, 70%, 80%, 90%, 95%, 96%, 97%, 98% or 99%.

In one embodiment, the fermentation broth includes (R)-3-hydroxybutyl (R)-3-hydroxybutanoate at a concentration of about 5%-15% by weight of (R)-3-hydroxybutyl (R)-3-hydroxybutanoate.

In one embodiment disclosed herein is a process for preparing (R)-3-hydroxybutyl (R)-3-hydroxybutanoate, the process including:
(a) isolating (R)-3-hydroxybutyric acid from a fermentation broth;
(b) reacting (R)-3-hydroxybutyric acid with ethanol to form a (R)-ethyl-3-hydroxybutyrate containing stream;
(c) isolating (R)-1,3-butanediol from a fermentation broth to form a (R)-1,3-butanediol containing stream;
(d) combining the (R)-ethyl-3-hydroxybutyrate containing stream with the (R)-1,3-butanediol containing stream in the presence of an esterifying agent to produce a (R)-3-hydroxybutyl (R)-3-hydroxybutanoate product stream; and
(e) purifying the (R)-3-hydroxybutyl (R)-3-hydroxybutanoate product stream.

In one embodiment, the (R)-3-hydroxybutyric acid is produced from glucose according to a fermentation process.

In one embodiment, (R)-1,3-butanediol is produced from glucose according to a fermentation process.

In one embodiment, the esterifying agent is an acid or an immobilized enzyme.

In one embodiment, the acid is selected from sulfuric acid, hydrochloric acid, acetic acid, benzoic acid, tosylic acid, candium(III) triflate, trifluoroacetic acid, phosphoric acid nitric acid, sulfamic acid, sulfonic acids, formic acid, acetic acid, lactic acid, propionic acid, oxalic acid, malonic acid, succinic acid, glutaric acid, or adipic acid.

In one embodiment, an immobilized enzyme is a lipase.

In one embodiment, the lipase is Novozyme 435, patatin, or Candida.

In one embodiment, the immobilized enzyme is an esterase. In one embodiment the esterase is a carboxylesterase.

In one embodiment, the purification includes a liquid-liquid extraction, distillation, filtration, or a combination thereof.

In one embodiment, the filtration is a microfiltration, nanofiltration, an ultrafiltration, or a combination thereof.

In one embodiment, step (d) is accomplished with reactive distillation.

Fermentation production of chemicals is a useful alternative to traditional synthesis using nonrenewable fossil fuel feedstocks. With the ability to utilize renewable feedstocks such as recycled biomass and the like, the process can prove more economical and environmentally sound than fossil fuel based production. In specific embodiments, the present disclosure provides methods for the production of (R)-3-hydroxybutyl (R)-3-hydroxybutanoate.

The cell-free broth, or liquid fraction, can be further processed by removal of salts. This can be achieved by several methods before or after removal of some or substantially all of the water from the fermentation broth. Salts are not often recovered for recycle in a fermentation process. Usually any salt recovery involves a salt form of a desired biosynthetic product such as lactate, citrate or other carboxylate product or ammonium salts of amine-containing products, rather than media salts and the like. The process described herein allows for recovery of media salts and optional recycle back into fermentation. The isolation process also involves removal of water, which can be reintroduced into the fermentation system. In the final purification, the compound produced by fermentation can be distilled, or recrystallized if solid, from the remaining liquid fraction after removal of cells, salts, and water. In the case of a liquid, the final purification can be accomplished by fractional distillation, for example.

In some embodiments disclosed herein is a process of isolating a water miscible compound of interest having a boiling point higher than water from a fermentation broth. The process includes (a) separating a liquid fraction enriched in the compound from a solid fraction that includes cells; (b) removing water from the liquid fraction; (c) removing salts from the liquid fraction, and (d) purifying the compound of interest by distillation or recrystallization. Steps (b) and (c) above may be performed in either order, or together.

In one specific embodiment, the compound of interest is (R)-3-hydroxybutyl (R)-3-hydroxybutanoate. (R)-3-hydroxybutyl (R)-3-hydroxybutanoate has a boiling point of about XX °C and is completely miscible with water in both a 50/50 (w/w) mixture, and a 60/40 (w/w) mixture of water/(R)-3-hydroxybutyl (R)-3-hydroxybutanoate.

As a neutral molecule, isolation by crystallization of a salt form is precluded. (R)-3-hydroxybutyl (R)-3-hydroxybutanoate has a molecular weight sufficiently low to pass through a nanofiltration membrane. Furthermore, the solubility of various fermentation media salts in pure (R)-3-hydroxybutyl (R)-3-hydroxybutanoate is relatively low.

In some embodiments disclosed herein is a process of isolating (R)-3-hydroxybutyl (R)-3-hydroxybutanoate from a fermentation broth that includes (a) separating a liquid fraction enriched in (R)-3-hydroxybutyl (R)-3-hydroxybutanoate from a solid fraction that includes cells; (b) removing water from the liquid fraction; (c) removing salts from the liquid fraction, and (d) purifying (R)-3-hydroxybutyl (R)-3-hydroxybutanoate.

In some embodiments disclosed herein is a process of isolating (R)-3-hydroxybutyl (R)-3-hydroxybutanoate from a fermentation broth. The process includes separating a liquid fraction enriched in (R)-3-hydroxybutyl (R)-3-hydroxybutanoate from a solid fraction that includes cells. Water is evaporated from the liquid fraction before or after separating salts from the liquid fraction. In some embodiments (R)-3-hydroxybutyl (R)-3-hydroxybutanoate is separated from salts that have crystallized after water removal as described further below. The salts have a low solubility in (R)-3-hydroxybutyl (R)-3-hydroxybutanoate such that the separated (R)-3-hydroxybutyl (R)-3-hydroxybutanoate is about 98% salt-free. In some embodiments, salts are separated by special filtration methods and/or ion exchange, or chromatographic methods prior to water removal as described further below.

In one embodiment disclosed herein is a process for preparing (3R)-hydroxybutyl (3R)-hydroxybutyrate, including the steps of:
(a) performing a first esterification between a C₁-C₃ alcohol and (R)-3-hydroxybutyric acid to form a first esterification product and water in a first esterification product stream
(b) subjecting the first esterification product stream to distillation to remove water to form a concentrated first esterification product stream;
(c) subjecting the concentrated first esterification product stream to distillation to form an enriched first esterification product stream and a heavies stream including (R)-3-hydroxybutyric acid
(d) subjecting the enriched first esterification product stream to a second esterification with (R)-1,3-butanediol to produce (3R)-hydroxybutyl (3R)-hydroxybutyrate and the C₁-C₃ alcohol in a second esterification product stream.

In one embodiment, the heavies stream including C₁-C₃ alcohol is recycled into the first esterification.

In one embodiment, the process further including subjecting the second esterification product stream to a purification procedure.

In one embodiment, the purification procedure includes distillation.

In one embodiment, the distillation includes:
(a) subjecting the second esterification product stream to a first column distillation procedure to remove materials with a boiling point lower than (3R)-hydroxybutyl (3R)-hydroxybutyrate from the second esterification product stream to produce a first (3R)-hydroxybutyl (3R)-hydroxybutyrate-containing product stream; and
(b) subjecting the first (3R)-hydroxybutyl (3R)-hydroxybutyrate-containing product stream to a second column distillation procedure to remove materials with boiling points higher than (3R)-hydroxybutyl (3R)-hydroxybutyrate as a first high-boilers stream, to produce a purified (3R)-hydroxybutyl (3R)-hydroxybutyrate product.

In one embodiment, the process further including:
(c) subjecting the first high-boilers stream to wiped-film evaporation (WFE) to produce a first WFE distillate and subjecting the first WFE distillate to step (b).

In one embodiment, the process further including:
(d) subjecting the first (3R)-hydroxybutyl (3R)-hydroxybutyrate-containing product stream, prior to performing step (b), to an intermediate column distillation procedure to remove materials with boiling points higher than (3R)-hydroxybutyl (3R)-hydroxybutyrate as a second high-boilers stream; and
(e) subjecting the second high-boilers stream to wiped-film evaporation (WFE) producing a second WFE distillate and subjecting the second WFE distillate to step (d).

In one embodiment, the C₁-C₃ alcohol generated during the second esterification is recovered and recycled.

In one embodiment, the C₁-C₃ alcohol generated during the second esterification is aqueous.

In one embodiment, the first column distillation procedure and second column distillation procedures are each performed at pressures equal to or less than atmospheric pressure.

In one embodiment, the pressure of the first column distillation procedure differs from the pressure of the second distillation procedure.

In one embodiment, the process further includes subjecting the purified (3R)-hydroxybutyl (3R)-hydroxybutyrate product to a polishing column.

In one embodiment, the polishing column is an ion exchange column.

In one embodiment, the ion exchange column uses an exchange resin that is an anion exchange resin.

In one embodiment, the ion exchange column uses an exchange resin that is a cation exchange resin.

In one embodiment, the purified (3R)-hydroxybutyl (3R)-hydroxybutyrate product is at least 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% pure.

In one embodiment, the first esterification is promoted with an acid.

In one embodiment, the acid is selected from sulfuric acid, hydrochloric acid, acetic acid, benzoic acid, tosylic acid, candium(III) triflate, trifluoroacetic acid, phosphoric acid nitric acid, sulfamic acid, sulfonic acids, formic acid, acetic acid, lactic acid, propionic acid, oxalic acid, malonic acid, succinic acid, glutaric acid, or adipic acid.

In one embodiment, the second esterification is promoted with an immobilized enzyme.

In one embodiment, the immobilized enzyme is a lipase.

In one embodiment, the lipase is selected from Novozyme 435, patatin, or Candida.

In one embodiment, the immobilized enzyme is an esterase. In one embodiment the esterase is a carboxylesterase.

In one embodiment, water is removed during the esterification reaction.

In one embodiment, the water removal during the esterification reaction is accomplished with reactive distillation.

In one embodiment provided herein is a process for preparing (3R)-hydroxybutyl (3R)-hydroxybutyrate, the process including:
(a) isolating (R)-3-hydroxybutyric acid from a fermentation broth;
(b) reacting (R)-3-hydroxybutyric acid with a C₁-C₃ alcohol to form a first esterification product stream;
(c) isolating (R)-1,3-butanediol from a fermentation broth to form a (R)-1,3-butanediol containing stream;
(d) combining the first esterification product stream with the (R)-1,3-butanediol containing stream in the presence of an esterifying agent to produce a (3R)-hydroxybutyl (3R)-hydroxybutyrate product stream; and
(e) purifying the (3R)-hydroxybutyl (3R)-hydroxybutyrate product stream.

In one embodiment, the (R)-3-hydroxybutyric acid from a fermentation broth is made by culturing a non-naturally occurring microbial organism.

In one embodiment, the non-naturally occurring microbial organism includes a (3R)-hydroxybutyrate pathway.

In one embodiment, the (3R)-hydroxybutyrate pathway includes a pathway selected from:
(1) 2B, 2C, and 2I;
(2) 2B, and 2H;
(3) 2J, 2K, 2C, and 2I;
(4) 2J, 2K, and 2H;
(5) 2A, 2B, 2C, and 2I;
(6) 2A, 2B, and 2H;
(7) 2A, 2J, 2K, 2C, and 2I;
(8) 2A, 2J, 2K, and 2H;
(9) 2E, 2F, 2B, 2C, and 2I;
(10) 2E, 2F, 2B, and 2H;
(11) 2E, 2F, 2J, 2K, 2C, and 2I;
(12) 2E, 2F, 2J, 2K, and 2H;
(13) 3A, 3B, and 3G;
(14) 3A, 3C, 2B, and 2H;
(15) 3A, 3C, 2B, 2C, and 2I;
(16) 3A, 3C, 2J, 2K, and 2H; and
(17) 3A, 3C, 2J, 2K, 2C, and 2I,
wherein 2A is an acetoacetyl-CoA thiolase, wherein 2B is a (3R)-hydroxybutyryl-CoA dehydrogenase, wherein 2C is a (3R)-hydroxybutyryl-CoA reductase, wherein 2E is an acetyl-CoA carboxylase, wherein 2F is an acetoacetyl-CoA synthase, wherein 2G is an acetoacetyl-CoA transferase, an acetoacetyl-CoA synthetase or an acetoacetyl-CoA hydrolase, wherein 2H is a (3R)-hydroxybutyryl-CoA transferase, a (3R)-hydroxybutyryl-CoA synthetase, or a (3R)-hydroxybutyryl-CoA hydrolase, wherein 2I is a (3R)-hydroxybutyraldehyde dehydrogenase, a (3R)-hydroxybutyraldehyde oxidase or a (3R)-hydroxybutyrate reductase, wherein 2J is a (3S)-hydroxybutyryl-CoA dehydrogenase, wherein 2K is a 3-hydroxybutyryl-CoA epimerase, wherein 3A is a 3-ketoacyl-ACP synthase, wherein 3B is an acetoacetyl-ACP reductase, wherein 3C is an acetoacetyl-CoA:ACP transferase, wherein 3G is an (3R)-hydroxybutyryl-ACP thioesterase.

In one embodiment, the (R)-1,3-butanediol from a fermentation broth is made by culturing a non-naturally occurring microbial organism.

In one embodiment, the non-naturally occurring microbial organism includes a (R)-1,3-butanediol pathway.

In one embodiment, the (R)-1,3-butanediol pathway includes a pathway selected from:
(1) 2B, 2C, and 2D;
(2) 2B, 2H, 2I,and 2D;
(3) 2J, 2K, 2C, and 2D;
(4) 2J, 2K, 2H, 2I,and 2D;
(5) 2A, 2B, 2C, and 2D;
(6) 2A, 2B, 2H, 2I,and 2D;
(7) 2A, 2J, 2K, 2C, and 2D;
(8) 2A, 2J, 2K, 2H, 2I,and 2D;
(9) 2E, 2F, 2B, 2C, and 2D;
(10) 2E, 2F, 2B, 2H, 2I,and 2D;
(11) 2E, 2F, 2J, 2K, 2C, and 2D;
(12) 2E, 2F, 2J, 2K, 2H, 2I,and 2D;
(13) 3A, 3B, and 3E;
(14) 3A, 3C, 2B, 2C, and 2D;
(15) 3A, 3C, 2B, 2H, 2I,and 2D;
(16) 3A, 3C, 2J, 2K, 2C, and 2D;
(17) 3A, 3C, 2J, 2K, 2H, 2I,and 2D;
(18) 3A, 3B, 3D, 2C, and 2D;
(19) 3A, 3B, 3D, 2H, 2I,and 2D;
(20) 3A, 3B, 3G, 2I,and 2D; and
(21) 3A, 3B, 3F, and 2D,
wherein 2A is an acetoacetyl-CoA thiolase, wherein 2B is a (3R)-hydroxybutyryl-CoA dehydrogenase, wherein 2C is a (3R)-hydroxybutyryl-CoA reductase, wherein 2D is a (3R)-hydroxybutyraldehyde reductase, wherein 2E is an acetyl-CoA carboxylase, wherein 2F is an acetoacetyl-CoA synthase, wherein 2H is a (3R)-hydroxybutyryl-CoA transferase, a (3R)-hydroxybutyryl-CoA synthetase, or a (3R)-hydroxybutyryl-CoA hydrolase, wherein 2I is a (3R)-hydroxybutyraldehyde dehydrogenase, (3R)-hydroxybutyraldehyde oxidase or (3R)-hydroxybutyrate reductase, wherein 2J is a (3 S)-hydroxybutyryl-CoA dehydrogenase, wherein 2K is a 3-hydroxybutyryl-CoA epimerase, wherein 3A is a 3-ketoacyl-ACP synthase, wherein 3B is an acetoacetyl-ACP reductase, wherein 3C is an acetoacetyl-CoA:ACP transferase, wherein 3D is a (3R)-hydroxybutyryl-CoA:ACP transferase, wherein 3E is a (3R)-hydroxybutyryl-ACP reductase (alcohol forming), wherein 3F is a (3R)-hydroxybutyryl-ACP reductase (aldehyde forming), wherein 3G is a (3R)-hydroxybutyryl-ACP thioesterase.

In one embodiment, (R)-3-hydroxybutyric acid is produced from glucose, xylose, arabinose, galactose, mannose, fructose, sucrose or starch according to a fermentation process.

In one embodiment, (R)-1,3-butanediol is produced from glucose, xylose, arabinose, galactose, mannose, fructose, sucrose or starch according to a fermentation process.

In one embodiment, the esterifying agent is an acid.

In one embodiment, the acid is selected from sulfuric acid, hydrochloric acid, acetic acid, benzoic acid, tosylic acid, candium(III) triflate, trifluoroacetic acid, phosphoric acid nitric acid, sulfamic acid, sulfonic acids, formic acid, acetic acid, lactic acid, propionic acid, oxalic acid, malonic acid, succinic acid, glutaric acid, or adipic acid.

In one embodiment, the esterifying agent is an immobilized enzyme.

In one embodiment, the immobilized enzyme is a lipase.

In one embodiment, the lipase is Novozyme 435, patatin, or Candida.

In one embodiment, the immobilized enzyme is an esterase. In one embodiment the esterase is a carboxylesterase.

In one embodiment, the purification includes a liquid-liquid extraction, distillation, filtration, or a combination thereof.

In one embodiment, the filtration is a microfiltration, nanofiltration, an ultrafiltration, or a combination thereof.

In one embodiment, the distillation includes:
(a) subjecting the (3R)-hydroxybutyl (3R)-hydroxybutyrate product stream to a first column distillation procedure to remove materials with a boiling point lower than (3R)-hydroxybutyl (3R)-hydroxybutyrate from the (3R)-hydroxybutyl (3R)-hydroxybutyrate product stream to produce a first (3R)-hydroxybutyl (3R)-hydroxybutyrate-containing product stream; and
(b) subjecting the first (3R)-hydroxybutyl (3R)-hydroxybutyrate-containing product stream to a second column distillation procedure to remove materials with boiling points higher than (3R)-hydroxybutyl (3R)-hydroxybutyrate as a first high-boilers stream, to produce a purified (3R)-hydroxybutyl (3R)-hydroxybutyrate product.

In one embodiment, the process further including:

(c) subjecting the first high-boilers stream to wiped-film evaporation (WFE) to produce a first WFE distillate and subjecting the first WFE distillate to step (b).

In one embodiment, the process further including:
(d) subjecting the first (3R)-hydroxybutyl (3R)-hydroxybutyrate-containing product stream, prior to performing step (b), to an intermediate column distillation procedure to remove materials with boiling points higher than (3R)-hydroxybutyl (3R)-hydroxybutyrate as a second high-boilers stream; and
(e) subjecting the second high-boilers stream to wiped-film evaporation (WFE) producing a second WFE distillate and subjecting the second WFE distillate to step (d).

In one embodiment, the first column distillation procedure and second column distillation procedures are each performed at pressures equal to or less than atmospheric pressure.

In one embodiment, the pressure of the first column distillation procedure differs from the pressure of the second distillation procedure.

In one embodiment, the process further includes subjecting the purified (3R)-hydroxybutyl (3R)-hydroxybutyrate product to a polishing column.

In one embodiment, the polishing column is an ion exchange column.

In one embodiment, the ion exchange column uses an exchange resin that is an anion exchange resin.

In one embodiment, the ion exchange column uses an exchange resin that is a cation exchange resin.

In one embodiment, the purified (3R)-hydroxybutyl (3R)-hydroxybutyrate is at least 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% pure.

In one embodiment, step (d) is accomplished with reactive distillation.

In one embodiment, C₁-C₃ alcohol generated in the (3R)-hydroxybutyl (3R)-hydroxybutyrate product stream is recovered and recycled.

In one embodiment, the C₁-C₃ alcohol generated during the second esterification is aqueous.

In one embodiment, isolating (R)-3-hydroxybutyric acid from a fermentation broth includes:
separating a liquid fraction enriched in (R)-3-hydroxybutyric acid from a solid fraction including cells, wherein said step of separating said liquid fraction includes one or more processes selected from the group consisting of microfiltration, ultrafiltration and nanofiltration;
removing salts from said liquid fraction, wherein salts are removed by ion exchange;
reducing water from said liquid fraction, wherein removing water is accomplished by evaporation; and
purifying (R)-3-hydroxybutyric acid from said liquid fraction.

In one embodiment, isolating (R)-1,3-butanediol from a fermentation broth includes
separating a liquid fraction enriched in (R)-1,3-butanediol from a solid fraction including cells, wherein said step of separating said liquid fraction includes one or more processes selected from the group consisting of microfiltration, ultrafiltration and nanofiltration;
removing salts from said liquid fraction, wherein salts are removed by ion exchange;
reducing water from said liquid fraction, wherein removing water is accomplished by evaporation; and
purifying (R)-1,3-butanediol from said liquid fraction.

In one embodiment, purifying (3R)-hydroxybutyl (3R)-hydroxybutyrate includes:
contacting the (3R)-hydroxybutyl (3R)-hydroxybutyrate product stream with an extraction solvent in a solvent contact column to make an extraction solvent enriched in (3R)-hydroxybutyl (3R)-hydroxybutyrate;
removing the extraction solvent enriched in (3R)-hydroxybutyl (3R)-hydroxybutyrate; and
subjecting the extraction solvent enriched in (3R)-hydroxybutyl (3R)-hydroxybutyrate to a purification process.

In one embodiment, the extraction solvent has a boiling point lower than (3R)-hydroxybutyl (3R)-hydroxybutyrate.

In one embodiment, the extraction solvent is 1-hexanol or 1-butanol.

In one embodiment, the purification process includes distillation.

In one embodiment, distillation includes:
(a) subjecting the extraction solvent enriched in (3R)-hydroxybutyl (3R)-hydroxybutyrate to a first column distillation procedure to remove materials with a boiling point lower than (3R)-hydroxybutyl (3R)-hydroxybutyrate from the extraction solvent enriched in (3R)-hydroxybutyl (3R)-hydroxybutyrate to produce a first (3R)-hydroxybutyl (3R)-hydroxybutyrate-containing product stream and a recovered extraction solvent stream; and
(b) subjecting the first (3R)-hydroxybutyl (3R)-hydroxybutyrate-containing product stream to a second column distillation procedure to remove materials with boiling points higher than (3R)-hydroxybutyl (3R)-hydroxybutyrate as a first high-boilers stream, to produce a purified (3R)-hydroxybutyl (3R)-hydroxybutyrate product.

In one embodiment, the process further including:

(c) subjecting the first high-boilers stream to wiped-film evaporation (WFE) to produce a first WFE distillate and subjecting the first WFE distillate to step (b).

In one embodiment, the process further including:
(d) subjecting the first (3R)-hydroxybutyl (3R)-hydroxybutyrate-containing product stream, prior to performing step (b), to an intermediate column distillation procedure to remove materials with boiling points higher than (3R)-hydroxybutyl (3R)-hydroxybutyrate as a second high-boilers stream; and
(e) subjecting the second high-boilers stream to wiped-film evaporation (WFE) producing a second WFE distillate and subjecting the second WFE distillate to step (d).

In one embodiment, the (3R)-hydroxybutyl (3R)-hydroxybutyrate is bioderived.

In one embodiment, the first column distillation procedure and second column distillation procedures are each performed at pressures equal to or less than atmospheric pressure.

In one embodiment, the pressure of the first column distillation procedure differs from the pressure of the second distillation procedure.

In one embodiment, the process further includes subjecting the purified (3R)-hydroxybutyl (3R)-hydroxybutyrate product to a polishing column.

In one embodiment, the polishing column is an ion exchange column.

In one embodiment, the ion exchange column uses an exchange resin that is an anion exchange resin.

In one embodiment, the ion exchange column uses an exchange resin that is a cation exchange resin.

In one embodiment, the recovered extraction solvent stream is recycled to the solvent contact column.

In one embodiment, the purified (3R)-hydroxybutyl (3R)-hydroxybutyrate product is greater than 90% (w/w), 91% (w/w), 92% (w/w), 93% (w/w), 94% (w/w), 95% (w/w), 96% (w/w), 97% (w/w), 98% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w) or 99.9% (w/w), (3R)-hydroxybutyl (3R)-hydroxybutyrate.

In one embodiment, recovery of (3R)-hydroxybutyl (3R)-hydroxybutyrate in the purified (3R)-hydroxybutyl (3R)-hydroxybutyrate product from the crude (3R)-hydroxybutyl (3R)-hydroxybutyrate mixture is greater than 40%, 50%, 60%, 70%, 80%, 90%, 95%, 96%, 97%, 98% or 99%.

In one embodiment, the purified (3R)-hydroxybutyl (3R)-hydroxybutyrate is enantiopure.

In one embodiment, the diameter of the solvent contact column is 1 cm to 10 m.

In one embodiment, the solvent contact column is static.

In one embodiment, the static solvent contact column is a structured packing column, random packing column, or a column including a sieve tray.

In one embodiment, the solvent contact column is agitated.

In one embodiment, the solvent contact column is agitated for a period of time.

In one embodiment, the agitation period is 1 second to 10 hours.

In one embodiment, the agitated solvent contact column is a rotating disc contactor or a pulsed column.

In one embodiment, the agitated solvent contact column is a Karr® column.

In one embodiment, the agitated solvent contact column is a Scheibel® column.

In one embodiment, the solvent contact column is a mixer-settler.

In one embodiment, the extraction solvent has a boiling point higher than (3R)-hydroxybutyl (3R)-hydroxybutyrate.

In one embodiment, the extraction solvent is tributyl phosphate.

In one embodiment, the purification process includes distillation.

In one embodiment, distillation includes:
(a) subjecting the extraction solvent enriched in (3R)-hydroxybutyl (3R)-hydroxybutyrate to a first column distillation procedure to remove materials with a boiling point lower than (3R)-hydroxybutyl (3R)-hydroxybutyrate from the extraction solvent enriched in (3R)-hydroxybutyl (3R)-hydroxybutyrate to produce a first (3R)-hydroxybutyl (3R)-hydroxybutyrate-containing product stream; and
(b) subjecting the first (3R)-hydroxybutyl (3R)-hydroxybutyrate-containing product stream to a second column distillation procedure to remove materials with boiling points higher than (3R)-hydroxybutyl (3R)-hydroxybutyrate as a first high-boilers stream, to produce a purified (3R)-hydroxybutyl (3R)-hydroxybutyrate product and a recovered extraction solvent stream.

In one embodiment, the process further including:

(c) subjecting the first high-boilers stream to wiped-film evaporation (WFE) to produce a first WFE distillate and subjecting the first WFE distillate to step (b).

In one embodiment, the process further including:
(d) subjecting the first (3R)-hydroxybutyl (3R)-hydroxybutyrate-containing product stream, prior to performing step (b), to an intermediate column distillation procedure to remove materials with boiling points higher than (3R)-hydroxybutyl (3R)-hydroxybutyrate as a second high-boilers stream; and
(e) subjecting the second high-boilers stream to wiped-film evaporation (WFE) producing a second WFE distillate and subjecting the second WFE distillate to step (d).

In one embodiment, the first column distillation procedure and second column distillation procedures are each performed at pressures equal to or less than atmospheric pressure.

In one embodiment, the pressure of the first column distillation procedure differs from the pressure of the second distillation procedure.

In one embodiment, the recovered extraction solvent stream is recycled to the solvent contact column.

In one embodiment, the process further includes subjecting the purified (3R)-hydroxybutyl (3R)-hydroxybutyrate product to a polishing column.

In one embodiment, the polishing column is an ion exchange column.

In one embodiment, the ion exchange column uses an exchange resin that is an anion exchange resin.

In one embodiment, the ion exchange column uses an exchange resin that is a cation exchange resin.

In one embodiment provided here, a process for preparing (3R)-hydroxybutyl (3R)-hydroxybutyrate, including the steps of
(a) performing an esterification reaction between ethyl (R)-3-hydroxybutanoate and (R)-1,3-butanediol in a reactor to form a product stream including (3R)-hydroxybutyl (3R)-hydroxybutyrate and ethanol;
(b) subjecting the product stream including (3R)-hydroxybutyl (3R)-hydroxybutyrate and ethanol to a first column distillation procedure to remove materials with a boiling point lower than (3R)-hydroxybutyl (3R)-hydroxybutyrate from the product stream to produce a first (3R)-hydroxybutyl (3R)-hydroxybutyrate-containing product stream;
(c) subjecting the first (3R)-hydroxybutyl (3R)-hydroxybutyrate-containing product stream to a second column distillation procedure to remove materials with boiling points higher than (3R)-hydroxybutyl (3R)-hydroxybutyrate as a first high-boilers stream, to produce a purified (3R)-hydroxybutyl (3R)-hydroxybutyrate product; and
(d) subjecting the first high-boilers stream to wiped-film evaporation (WFE) to produce a first WFE distillate and subjecting the first WFE distillate to step (c).

In one embodiment, the esterification reaction is promoted with an acid.

In one embodiment, the acid is selected from sulfuric acid, hydrochloric acid, acetic acid, benzoic acid, tosylic acid, candium(III) triflate, trifluoroacetic acid, phosphoric acid nitric acid, sulfamic acid, sulfonic acids, formic acid, acetic acid, lactic acid, propionic acid, oxalic acid, malonic acid, succinic acid, glutaric acid, or adipic acid.

In one embodiment, the second esterification is promoted with an immobilized enzyme.

In one embodiment, the immobilized enzyme is a lipase.

In one embodiment, the lipase is selected from Novozyme 435, patatin, or Candida.

In one embodiment, the immobilized enzyme is an esterase. In one embodiment the esterase is a carboxylesterase.

In one embodiment, the ethanol generated during the second esterification is recovered and recycled.

In one embodiment, the ethanol generated during the second esterification is aqueous.

In one embodiment, the reactor operates at a temperature of 0° C. to 120° C.

In one embodiment, the reactor operates at a temperature of 10° C. to 50° C.

In one embodiment, the reactor operates under reduced pressure. In one embodiment, the pressure is between 5 and 400 mmHg.

In one embodiment, the reactor operates under positive pressure. In one embodiment, the pressure is between 1 and 2 atmospheres.

In one embodiment, the process further including:
(e) subjecting the first (3R)-hydroxybutyl (3R)-hydroxybutyrate-containing product stream, prior to performing step (c), to an intermediate column distillation procedure to remove materials with boiling points higher than (3R)-hydroxybutyl (3R)-hydroxybutyrate as a second high-boilers stream; and
(f) subjecting the second high-boilers stream to wiped-film evaporation (WFE) producing a second WFE distillate and subjecting the second WFE distillate to step (e).

In one embodiment, the purified (3R)-hydroxybutyl (3R)-hydroxybutyrate is at least 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% pure.

In one embodiment, the first column distillation procedure and second column distillation procedures are each performed at pressures equal to or less than atmospheric pressure.

In one embodiment, the pressure of the first column distillation procedure differs from the pressure of the second distillation procedure.

In one embodiment, the process further includes subjecting the purified (3R)-hydroxybutyl (3R)-hydroxybutyrate product to a polishing column.

In one embodiment, the polishing column is an ion exchange column.

In one embodiment, the ion exchange column uses an exchange resin that is an anion exchange resin.

In one embodiment, the ion exchange column uses an exchange resin that is a cation exchange resin.

In one embodiment, the materials with a boiling point lower than (3R)-hydroxybutyl (3R)-hydroxybutyrate include (R)-3-hydroxybutanoate and (R)-1,3-butanediol.

In one embodiment, the (R)-3-hydroxybutanoate and (R)-1,3-butanediol are recycled back into the reactor.

In one embodiment, the ethanol is removed during the esterification reaction.

In one embodiment, ethanol removal during the esterification reaction is accomplished with reactive distillation.

In one embodiment, a process for preparing (3R)-hydroxybutyl (3R)-hydroxybutyrate, including the steps of
(a) isolating (R)-3-hydroxybutanoic acid from a fermentation broth;
(b) performing an esterification reaction between (R)-3-hydroxybutanoic acid and (R)-1,3-butanediol in a reactor to form a product stream including (3R)-hydroxybutyl (3R)-hydroxybutyrate; and
(c) subjecting the product stream including (3R)-hydroxybutyl (3R)-hydroxybutyrate to a first column distillation procedure to remove materials with a boiling point lower than (3R)-hydroxybutyl (3R)-hydroxybutyrate from the product stream to produce a first (3R)-hydroxybutyl (3R)-hydroxybutyrate-containing product stream;
(d) subjecting the first (3R)-hydroxybutyl (3R)-hydroxybutyrate-containing product stream to a second column distillation procedure to remove materials with boiling points higher than (3R)-hydroxybutyl (3R)-hydroxybutyrate as a first high-boilers stream, to produce a purified (3R)-hydroxybutyl (3R)-hydroxybutyrate product; and
(e) subjecting the first high-boilers stream to wiped-film evaporation (WFE) to produce a first WFE distillate and subjecting the first WFE distillate to step (d).

In one embodiment, the esterification reaction is promoted with an acid.

In one embodiment, the acid is selected from sulfuric acid, hydrochloric acid, acetic acid, benzoic acid, tosylic acid, candium(III) triflate, trifluoroacetic acid, phosphoric acid nitric acid, sulfamic acid, sulfonic acids, formic acid, acetic acid, lactic acid, propionic acid, oxalic acid, malonic acid, succinic acid, glutaric acid, or adipic acid.

In one embodiment, the second esterification is promoted with an immobilized enzyme.

In one embodiment, the immobilized enzyme is a lipase.

In one embodiment, the lipase is selected from Novozyme 435, patatin, or Candida.

In one embodiment, the immobilized enzyme is an esterase. In one embodiment the esterase is a carboxylesterase.

In one embodiment, the reactor operates at a temperature of 0° C. to 120° C.

In one embodiment, the reactor operates at a temperature of 10° C. to 50° C.

In one embodiment, the reactor operates under reduced pressure. In one embodiment, the pressure is between 5 and 400 mmHg.

In one embodiment, the reactor operates under positive pressure. In one embodiment, the pressure is between 1 and 2 atmospheres.

In one embodiment the process further including:
(f) subjecting the first (3R)-hydroxybutyl (3R)-hydroxybutyrate-containing product stream, prior to performing step (d), to an intermediate column distillation procedure to remove materials with boiling points higher than (3R)-hydroxybutyl (3R)-hydroxybutyrate as a second high-boilers stream; and
(g) subjecting the second high-boilers stream to wiped-film evaporation (WFE) producing a second WFE distillate and subjecting the second WFE distillate to step (f).

In one embodiment, the purified (3R)-hydroxybutyl (3R)-hydroxybutyrate is at least 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% pure.

In one embodiment, the first column distillation procedure and second column distillation procedures are each performed at pressures equal to or less than atmospheric pressure.

In one embodiment, the pressure of the first column distillation procedure differs from the pressure of the second distillation procedure.

In one embodiment, the process further includes subjecting the purified (3R)-hydroxybutyl (3R)-hydroxybutyrate product to a polishing column.

In one embodiment, the polishing column is an ion exchange column.

In one embodiment, the ion exchange column uses an exchange resin that is an anion exchange resin.

In one embodiment, the ion exchange column uses an exchange resin that is a cation exchange resin.

In one embodiment, the materials with a boiling point lower than (3R)-hydroxybutyl (3R)-hydroxybutyrate include (R)-3-hydroxybutanoate and (R)-1,3-butanediol.

In one embodiment, the (R)-3-hydroxybutanoate and (R)-1,3-butanediol are recycled back into the reactor.

In one embodiment, the water is removed during the esterification reaction.

In one embodiment, the water removal during the esterification reaction is accomplished with reactive distillation.

In one embodiment provided herein, a process of isolating (3R)-hydroxybutyl (3R)-hydroxybutyrate from a fermentation broth, the process including:
separating a liquid fraction enriched in (3R)-hydroxybutyl (3R)-hydroxybutyrate from a solid fraction of the fermentation broth including cells;
contacting the liquid fraction enriched in (3R)-hydroxybutyl (3R)-hydroxybutyrate with an extraction solvent in a solvent contact column to make an extraction solvent enriched in (3R)-hydroxybutyl (3R)-hydroxybutyrate;
removing the extraction solvent enriched in (3R)-hydroxybutyl (3R)-hydroxybutyrate; and
subjecting the extraction solvent enriched in (3R)-hydroxybutyl (3R)-hydroxybutyrate to a purification process.

In one embodiment, the purification process includes filtration or centrifugation.

In one embodiment, the centrifugation is accomplished with a disc-stack centrifuge or a decanter centrifuge.

In one embodiment, the filtration includes one or more processes selected from the group consisting of microfiltration, ultrafiltration and nanofiltration

In one embodiment, ultrafiltration includes filtering through a membrane having a pore size from about 0.005 to about 0.1 microns.

In one embodiment, microfiltration includes filtering through a membrane having a pore size from about 0.1 microns to about 5.0 microns.

In one embodiment, nanofiltration includes filtering through a membrane having a pore size from about 0.0005 microns to about 0.005 microns.

In one embodiment, the extraction solvent has a boiling point lower than (3R)-hydroxybutyl (3R)-hydroxybutyrate.

In one embodiment, the extraction solvent is 1-hexanol or 1-butanol.

In one embodiment, purifying the extraction solvent enriched in (3R)-hydroxybutyl (3R)-hydroxybutyrate is accomplished by distillation.

In one embodiment, distillation includes:
(a) subjecting the extraction solvent enriched in (3R)-hydroxybutyl (3R)-hydroxybutyrate to a first column distillation procedure to remove materials with a boiling point lower than (3R)-hydroxybutyl (3R)-hydroxybutyrate from the extraction solvent enriched in (3R)-hydroxybutyl (3R)-hydroxybutyrate to produce a first (3R)-hydroxybutyl (3R)-hydroxybutyrate-containing product stream and a recovered extraction solvent stream; and
(b) subjecting the first (3R)-hydroxybutyl (3R)-hydroxybutyrate-containing product stream to a second column distillation procedure to remove materials with boiling points higher than (3R)-hydroxybutyl (3R)-hydroxybutyrate as a first high-boilers stream, to produce a purified (3R)-hydroxybutyl (3R)-hydroxybutyrate product.

In one embodiment the process further including:

(c) subjecting the first high-boilers stream to wiped-film evaporation (WFE) to produce a first WFE distillate and subjecting the first WFE distillate to step (b).

In one embodiment the process further including:
(d) subjecting the first (3R)-hydroxybutyl (3R)-hydroxybutyrate-containing product stream, prior to performing step (b), to an intermediate column distillation procedure to remove materials with boiling points higher than (3R)-hydroxybutyl (3R)-hydroxybutyrate as a second high-boilers stream; and
(e) subjecting the second high-boilers stream to wiped-film evaporation (WFE) producing a second WFE distillate and subjecting the second WFE distillate to step (d).

In one embodiment, wherein the (3R)-hydroxybutyl (3R)-hydroxybutyrate is bioderived.

In one embodiment, the first column distillation procedure and second column distillation procedures are each performed at pressures equal to or less than atmospheric pressure.

In one embodiment, the pressure of the first column distillation procedure differs from the pressure of the second distillation procedure.

In one embodiment, the process further includes subjecting the purified (3R)-hydroxybutyl (3R)-hydroxybutyrate product to a polishing column.

In one embodiment, the polishing column is an ion exchange column.

In one embodiment, the ion exchange column uses an exchange resin that is an anion exchange resin.

In one embodiment, the ion exchange column uses an exchange resin that is a cation exchange resin.

In one embodiment, the recovered extraction solvent stream is recycled to the solvent contact column.

In one embodiment, the fermentation broth includes (3R)-hydroxybutyl (3R)-hydroxybutyrate at a concentration of about 1%-50% by weight of (3R)-hydroxybutyl (3R)-hydroxybutyrate.

In one embodiment, the purified (3R)-hydroxybutyl (3R)-hydroxybutyrate is at least 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% pure.

In one embodiment, the solvent contact column is operated at room temperature, atmospheric pressure or both.

In one embodiment, the diameter of the solvent contact column is 1 cm to 10 m.

In one embodiment, the solvent contact column is static.

In one embodiment, the static solvent contact column is a structured packing column, random packing column, or a column including a sieve tray.

In one embodiment, the solvent contact column is agitated.

In one embodiment, the solvent contact column is agitated for a period of time.

In one embodiment, the agitation period is 1 second to 10 hours.

In one embodiment, the agitated solvent contact column is a rotating disc contactor or a pulsed column.

In one embodiment, the agitated solvent contact column is a Karr® column.

In one embodiment, the agitated solvent contact column is a Scheibel® column.

In one embodiment, the solvent contact column is a mixer-settler.

In one embodiment, the fermentation broth includes (3R)-hydroxybutyl (3R)-hydroxybutyrate at a concentration of about 5%-15% by weight of (3R)-hydroxybutyl (3R)-hydroxybutyrate.

In one embodiment, the purified (3R)-hydroxybutyl (3R)-hydroxybutyrate product is greater than 90% (w/w), 91% (w/w), 92% (w/w), 93% (w/w), 94% (w/w), 95% (w/w), 96% (w/w), 97%, (w/w) 98% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w) or 99.9% (w/w), (3R)-hydroxybutyl (3R)-hydroxybutyrate.

In one embodiment, the recovery of (3R)-hydroxybutyl (3R)-hydroxybutyrate in the purified (3R)-hydroxybutyl (3R)-hydroxybutyrate product (3R)-hydroxybutyl (3R)-hydroxybutyrate is greater than 40%, 50%, 60%, 70%, 80%, 90%, 95%, 96%, 97%, 98% or 99%.

In one embodiment, the extraction solvent has a boiling point higher than (3R)-hydroxybutyl (3R)-hydroxybutyrate.

In one embodiment, the extraction solvent is tributyl phosphate.

In one embodiment, purifying the extraction solvent enriched in (3R)-hydroxybutyl (3R)-hydroxybutyrate is accomplished by distillation.

In one embodiment, distillation includes:
(a) subjecting the extraction solvent enriched in (3R)-hydroxybutyl (3R)-hydroxybutyrate to a first column distillation procedure to remove materials with a boiling point lower than (3R)-hydroxybutyl (3R)-hydroxybutyrate from the extraction solvent enriched in (3R)-hydroxybutyl (3R)-hydroxybutyrate to produce a first (3R)-hydroxybutyl (3R)-hydroxybutyrate-containing product stream; and
(b) subjecting the first (3R)-hydroxybutyl (3R)-hydroxybutyrate-containing product stream to a second column distillation procedure to remove materials with boiling points higher than (3R)-hydroxybutyl (3R)-hydroxybutyrate as a first high-boilers stream, to produce a purified (3R)-hydroxybutyl (3R)-hydroxybutyrate product and a recovered extraction solvent stream.

In one embodiment the process further including:

(c) subjecting the first high-boilers stream to wiped-film evaporation (WFE) to produce a first WFE distillate and subjecting the first WFE distillate to step (b).

In one embodiment the process further including:
(d) subjecting the first (3R)-hydroxybutyl (3R)-hydroxybutyrate-containing product stream, prior to performing step (b), to an intermediate column distillation procedure to remove materials with boiling points higher than (3R)-hydroxybutyl (3R)-hydroxybutyrate as a second high-boilers stream; and
(e) subjecting the second high-boilers stream to wiped-film evaporation (WFE) producing a second WFE distillate and subjecting the second WFE distillate to step (d).

In one embodiment, the first column distillation procedure and second column distillation procedures are each performed at pressures equal to or less than atmospheric pressure.

In one embodiment, the pressure of the first column distillation procedure differs from the pressure of the second distillation procedure.

In one embodiment, the recovered extraction solvent stream is recycled to the solvent contact column.

In one embodiment, the process further includes subjecting the purified (3R)-hydroxybutyl (3R)-hydroxybutyrate product to a polishing column.

In one embodiment, the polishing column is an ion exchange column.

In one embodiment, the ion exchange column uses an exchange resin that is an anion exchange resin.

In one embodiment, the ion exchange column uses an exchange resin that is a cation exchange resin.

In one embodiment provided herein, a process of isolating (3R)-hydroxybutyl (3R)-hydroxybutyrate from a fermentation broth including
(a) separating a liquid fraction enriched in (3R)-hydroxybutyl (3R)-hydroxybutyrate from a solid fraction including cells, wherein said step of separating said liquid fraction includes one or more processes selected from the group consisting of microfiltration, ultrafiltration and nanofiltration;
(b) removing salts from said liquid fraction, wherein salts are removed by ion exchange;
(c) reducing water from said liquid fraction, wherein removing water is accomplished by evaporation, to form a concentrated liquid fraction;
(d) subjecting the concentrated liquid fraction to a first column distillation procedure to remove materials with boiling points higher than (3R)-hydroxybutyl (3R)-hydroxybutyrate from the concentrated liquid fraction containing (3R)-hydroxybutyl (3R)-hydroxybutyrate to produce a first (3R)-hydroxybutyl (3R)-hydroxybutyrate-containing product stream and a high-boilers stream;
(e) subjecting the first (3R)-hydroxybutyl (3R)-hydroxybutyrate-containing product stream to a second column distillation procedure to remove materials with boiling points lower than (3R)-hydroxybutyl (3R)-hydroxybutyrate as a first low-boilers stream, to produce a purified (3R)-hydroxybutyl (3R)-hydroxybutyrate product; and
(f) subjecting the high-boilers stream to wiped-film evaporation (WFE) to produce a first WFE distillate and subjecting the first WFE distillate to step (d).

In one embodiment, microfiltration includes filtering through a membrane having a pore size from about 0.1 microns to about 5.0 microns

In one embodiment, ultrafiltration includes filtering through a membrane having a pore size from about 0.005 to about 0.1 microns.

In one embodiment, nanofiltration includes filtering through a membrane having a pore size from about 0.0005 microns to about 0.005 microns.

In one embodiment, the evaporation is accomplished with an evaporator system.

In one embodiment, said evaporator system includes an evaporator selected from the group consisting of a falling film evaporator, a short path falling film evaporator, a forced circulation evaporator, a plate evaporator, a circulation evaporator, a fluidized bed evaporator, a rising film evaporator, a counterflow-trickle evaporator, a stirrer evaporator, and a spiral tube evaporator.

In one embodiment, the reduction of water is from about 85% by weight to about 15% by weight.

In one embodiment, the (3R)-hydroxybutyl (3R)-hydroxybutyrate is bioderived.

In one embodiment, the first column distillation procedure and second column distillation procedures are each performed at pressures equal to or less than atmospheric pressure.

In one embodiment, the pressure of the first column distillation procedure differs from the pressure of the second distillation procedure.

In one embodiment, the purified (3R)-hydroxybutyl (3R)-hydroxybutyrate product is greater than 90% (w/w), 91% (w/w), 92% (w/w), 93% (w/w), 94% (w/w), 95% (w/w), 96% (w/w), 97%, (w/w) 98% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w) or 99.9% (w/w), (3R)-hydroxybutyl (3R)-hydroxybutyrate.

In one embodiment, recovery of (3R)-hydroxybutyl (3R)-hydroxybutyrate in the purified (3R)-hydroxybutyl (3R)-hydroxybutyrate product (3R)-hydroxybutyl (3R)-hydroxybutyrate is greater than 40%, 50%, 60%, 70%, 80%, 90%, 95%, 96%, 97%, 98% or 99%.

In one embodiment, the fermentation broth includes (3R)-hydroxybutyl (3R)-hydroxybutyrate at a concentration of about 5%-15% by weight of (3R)-hydroxybutyl (3R)-hydroxybutyrate.

As used herein, “isolating” refers to a process that includes purification steps to obtain a substantially purified compound of interest. In particular embodiments, a compound of interest includes (R)-3-hydroxybutyl (R)-3-hydroxybutanoate. A substantially purified compound includes those that are at least 98% salt free, in some embodiments, at least 99% salt free in other embodiments, and at least 99.5% salt free in still other embodiments. A substantially purified compound also includes those that are also free of other impurities in addition to salts such that the compound of interest is at least 98% pure in some embodiments, at least 99% pure in other embodiments, and at least 99.5% pure in still further embodiments.

As used herein, the term “liquid fraction” refers to a centrate or supernatant liquid obtained upon removal of solid mass from the fermentation broth. Solid mass removal includes, some, substantially all, or all of a solid mass. For example, in centrifugation, the liquid fraction is the centrate or supernatant which is separated from the solids. The liquid fraction is also the portion that is the permeate or supernatant obtained after filtration through a membrane. The liquid fraction is also the portion that is the filtrate or supernatant obtained after one or more filtration methods have been applied.

As used herein, the term “solid fraction” refers to a portion of the fermentation broth containing insoluble materials. Such insoluble materials include, for example, cells, cell debris, precipitated proteins, fines, and the like. Fines refer to small, usually amorphous solids. Fines can also be created during crystallization or during removal of water from the fermentation broth. Fines can be made up of a compound of interest which can be dissolved and recrystallized out. Fines can include portions of the solid fraction that are too small to be captured in a membrane filtration.

As used herein, the term “bioderived” means produced from or synthesized by a biological organism and can be considered a renewable resource since it can be generated by a biological organism. Such a biological organism, in particular the microbial organisms disclosed herein, can utilize feedstock or biomass, such as, sugars or carbohydrates obtained from an agricultural, plant, bacterial, or animal source; or other renewable sources such as synthesis gas (CO, CO₂ and/or H₂). Coal products can also be used as a carbon source for a biological organism to synthesize a biobased product. Alternatively, the biological organism can utilize atmospheric carbon. As used herein, the term “biobased” means a product as described above that is composed, in whole or in part, of a bioderived compound. A biobased or bioderived product is in contrast to a petroleum derived product, wherein such a product is derived from or chemically synthesized from petroleum or a petrochemical feedstock.

As used herein, the term “non-naturally occurring” when used in reference to a microbial organism or microorganism of the invention is intended to mean that the microbial organism has at least one genetic alteration not normally found in a naturally occurring strain of the referenced species, including wild-type strains of the referenced species. Genetic alterations include, for example, modifications introducing expressible nucleic acids encoding metabolic polypeptides, other nucleic acid additions, nucleic acid deletions and/or other functional disruption of the microbial organism’s genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon. Exemplary metabolic polypeptides include enzymes or proteins within a (R)-3-hydroxybutyl (R)-3-hydroxybutanoate biosynthetic pathway.

A metabolic modification refers to a biochemical reaction that is altered from its naturally occurring state. Therefore, non-naturally occurring microorganisms can have genetic modifications to nucleic acids encoding metabolic polypeptides, or functional fragments thereof. Exemplary metabolic modifications are disclosed herein.

As used herein, the term “isolated” when used in reference to a microbial organism is intended to mean an organism that is substantially free of at least one component as the referenced microbial organism is found in nature. The term includes a microbial organism that is removed from some or all components as it is found in its natural environment. The term also includes a microbial organism that is removed from some or all components as the microbial organism is found in non-naturally occurring environments. Therefore, an isolated microbial organism is partly or completely separated from other substances as it is found in nature or as it is grown, stored or subsisted in non-naturally occurring environments. Specific examples of isolated microbial organisms include partially pure microbes, substantially pure microbes and microbes cultured in a medium that is non-naturally occurring.

As used herein, the terms “microbial,” “microbial organism” or “microorganism” are intended to mean any organism that exists as a microscopic cell that is included within the domains of archaea, bacteria or eukarya. Therefore, the term is intended to encompass prokaryotic or eukaryotic cells or organisms having a microscopic size and includes bacteria, archaea and eubacteria of all species as well as eukaryotic microorganisms such as yeast and fungi. The term also includes cell cultures of any species that can be cultured for the production of a biochemical.

As used herein, the term “CoA” or “coenzyme A” is intended to mean an organic cofactor or prosthetic group (nonprotein portion of an enzyme) whose presence is required for the activity of many enzymes (the apoenzyme) to form an active enzyme system. Coenzyme A functions in certain condensing enzymes, acts in acetyl or other acyl group transfer and in fatty acid synthesis and oxidation, pyruvate oxidation and in other acetylation.

As used herein, the term “substantially anaerobic” when used in reference to a culture or growth condition is intended to mean that the amount of oxygen is less than about 10% of saturation for dissolved oxygen in liquid media. The term also is intended to include sealed chambers of liquid or solid medium maintained with an atmosphere of less than about 1% oxygen.

“Exogenous” as it is used herein is intended to mean that the referenced molecule or the referenced activity is introduced into the host microbial organism. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the microbial organism. When used in reference to a biosynthetic activity, the term refers to an activity that is introduced into the host reference organism. The source can be, for example, a homologous or heterologous encoding nucleic acid that expresses the referenced activity following introduction into the host microbial organism. Therefore, the term “endogenous” refers to a referenced molecule or activity that is present in the host. Similarly, the term when used in reference to expression of an encoding nucleic acid refers to expression of an encoding nucleic acid contained within the microbial organism. The term “heterologous” refers to a molecule or activity derived from a source other than the referenced species whereas “homologous” refers to a molecule or activity derived from the host microbial organism. Accordingly, exogenous expression of an encoding nucleic acid of the invention can utilize either or both a heterologous or homologous encoding nucleic acid.

It is understood that when more than one exogenous nucleic acid is included in a microbial organism that the more than one exogenous nucleic acids refers to the referenced encoding nucleic acid or biosynthetic activity, as discussed above. It is further understood, as disclosed herein, that such more than one exogenous nucleic acids can be introduced into the host microbial organism on separate nucleic acid molecules, on polycistronic nucleic acid molecules, or a combination thereof, and still be considered as more than one exogenous nucleic acid. For example, as disclosed herein a microbial organism can be engineered to express two or more exogenous nucleic acids encoding a desired pathway enzyme or protein. In the case where two exogenous nucleic acids encoding a desired activity are introduced into a host microbial organism, it is understood that the two exogenous nucleic acids can be introduced as a single nucleic acid, for example, on a single plasmid, on separate plasmids, can be integrated into the host chromosome at a single site or multiple sites, and still be considered as two exogenous nucleic acids. Similarly, it is understood that more than two exogenous nucleic acids can be introduced into a host organism in any desired combination, for example, on a single plasmid, on separate plasmids, can be integrated into the host chromosome at a single site or multiple sites, and still be considered as two or more exogenous nucleic acids, for example three exogenous nucleic acids. Thus, the number of referenced exogenous nucleic acids or biosynthetic activities refers to the number of encoding nucleic acids or the number of biosynthetic activities, not the number of separate nucleic acids introduced into the host organism.

As used herein, the term “salts,” used interchangeably with media salts and fermentation media salts, refers to the dissolved ionic compounds used in a fermentation broth. Salts in a fermentation broth can include, for example, sodium chloride, potassium chloride, calcium chloride, ammonium chloride, magnesium sulfate, ammonium sulfate, and buffers such as sodium and/or potassium and/or ammonium salts of phosphate, citrate, acetate, and borate.

As used herein, the term “substantially all” when used in reference to removal of water or salts refers to the removal of at least 95% of water or salts. “Substantially all” can also include at least 96%, 97%, 98%, 99%, or 99.9% removal or any value in between.

As used herein, the term “gene disruption” or grammatical equivalents thereof, is intended to mean a genetic alteration that renders the encoded gene product inactive. The genetic alteration can be, for example, deletion of the entire gene, deletion of a regulatory sequence required for transcription or translation, deletion of a portion of the gene with results in a truncated gene product or by any of various mutation strategies that inactivate the encoded gene product. One particularly useful method of gene disruption is complete gene deletion because it reduces or eliminates the occurrence of genetic reversions in non-naturally occurring microorganisms.

As used herein, the term “microorganism” is intended to mean a prokaryotic or eukaryotic cell or organism having a microscopic size. The term is intended to include bacteria of all species and eukaryotic organisms such as yeast and fungi. The term also includes cell cultures of any species that can be cultured for the production of a biochemical.

As used herein, the term “(R)-3-hydroxybutyl (R)-3-hydroxybutanoate-producing microorganism” is intended to mean a microorganism engineered to biosynthesize (R)-3-hydroxybutyl (R)-3-hydroxybutanoate in useful amounts. The engineered organism can include gene insertions, which includes plasmid inserts and/or chromosomal insertions. The engineered organism can also include gene disruptions to further optimize carbon flux through the desired pathways for production of (R)-3-hydroxybutyl (R)-3-hydroxybutanoate. (R)-3-hydroxybutyl (R)-3-hydroxybutanoate-producing organisms can include combination of insertions and deletions.

“Exogenous” as it is used herein is intended to mean that the referenced molecule or the referenced activity is introduced into the host microbial organism. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the microbial organism. When used in reference to a biosynthetic activity, the term refers to an activity that is introduced into the host reference organism. The source can be, for example, a homologous or heterologous encoding nucleic acid that expresses the referenced activity following introduction into the host microbial organism. Therefore, the term “endogenous” refers to a referenced molecule or activity that is present in the host. Similarly, the term when used in reference to expression of an encoding nucleic acid refers to expression of an encoding nucleic acid contained within the microbial organism. The term “heterologous” refers to a molecule or activity derived from a source other than the referenced species whereas “homologous” refers to a molecule or activity derived from the host microbial organism. Accordingly, exogenous expression of an encoding nucleic acid can utilize either or both a heterologous or homologous encoding nucleic acid.

In some embodiments disclosed herein is a process of purifying a compound of interest from a fermentation broth. Applicable compounds include those having a boiling point higher than water and a low salt solubility. An exemplary compound of interest is (R)-3-hydroxybutyl (R)-3-hydroxybutanoate. The process includes separating a liquid fraction which contains the product of interest, from a solid fraction which includes the cells mass. The product of interest can be any compound having a higher boiling point than water. The cell mass includes the microbial organisms used in the production of the compound of interest. The solid fraction also includes cell debris, fines, proteins, and other insoluble materials from the fermentation.

The isolation process also includes removing the salts and water from the liquid fraction. The order in which they are removed is inconsequential. In some embodiments, there can be partial removal of salts, followed by removal of substantially all the water, and then the remaining salts. In other embodiments, there can be partial removal of water, followed by removal of substantially all of the salts, and then the remaining water. In other embodiments, water can be partially removed prior to separation of the solid fraction from the fermentation broth. In still other embodiments, final removal of substantially all the water can be done as part of the purification steps, for example by distillation. (R)-3-hydroxybutyl (R)-3-hydroxybutanoate can be separated from salts by evaporation of the water from the liquid fraction. In some embodiments, (R)-3-hydroxybutyl (R)-3-hydroxybutanoate is a least 98% salt free upon separation of (R)-3-hydroxybutyl (R)-3-hydroxybutanoate from salts crystallized or precipitated by water removal. Other methods can be employed to remove salts even after removal of substantially all the water.

Eventually when the salts and water have been removed, the remaining liquid or solid can undergo final purification. When the product of interest is a liquid, purification can be accomplished by distillation including by fractional distillation or multiple distillation, for example. When the product of interest is a solid, purification can be accomplished by recrystallization.

In some embodiments, a process of isolating a compound of interest, including (R)-3-hydroxybutyl (R)-3-hydroxybutanoate, from a fermentation broth involves separating a liquid fraction enriched in the compound of interest from a solid fraction that includes cells. In separating a liquid fraction enriched in the compound of interest, any amount of the fermentation broth can be processed including 1%, 5%, 10%, 20%, 30%, 40%, 50%, 60%, 70%, 80%, 90%, including up to the entirety of the volume of the fermentation broth and all values in between, and further including volumes less than 1% of the total volume of the fermentation broth. One skilled in the art will recognize that the amount of fermentation broth processed can depend on the type of fermentation process, such as batch, fed batch, or continuous, as detailed below. Separation of solids which includes cells and other solid byproducts and impurities from the fermentation broth can be accomplished by centrifugation, filtration, or a combination of these methods.

In some embodiments, centrifugation can be used to provide a liquid fraction including the compound of interest, such as (R)-3-hydroxybutyl (R)-3-hydroxybutanoate, substantially free of solids including the cell mass. Depending on the centrifuge configuration and size, operating speeds can vary between 500 to 12,000 rpm which produce a centrifugal force of up to 15,000 times the force of gravity. Many centrifuge configurations for removal of cells and solids from a fermentation broth are known in the art.

A disc stack centrifuge separates solids and one or two liquid phases from each other, typically in a continuous process. The denser solids are forced outwards by centrifugal forces while the less dense liquid phases form inner concentric layers. By inserting special plates (disc stack) separation efficiency is increased. The solids can be removed manually, intermittently or continuously. In accordance with some embodiments, the cell mass can be introduced back into the fermentation. In a typical disc-stack centrifuge apparatus, the liquid phase overflows in an outlet area on top of a bowl into a separate chamber.

During operation of a disc-stack centrifuge, feed is introduced at the axis of the bowl, accelerated to speed, often by a radial vane assembly, and flows through a stack of closely spaced conical disks. Disk spacing is often between 0.5 to 3 mm in order to reduce the distance needed for separating settling particles from the fluid. The disc angle is often between 40 and 50 degrees to facilitate solids transport down the disk surface into the solids holding space.

The separating mechanism is based on the settling of solids under the influence of centrifugal force against the underside of the disks and slide down the disk into the solids hold space. Concurrently the clarified fluid moves up the channel between the disks and leaves the centrifuge via a centripetal pump. The settled solids are discharged either continuously though nozzles or intermittently through ports at the bowl periphery.

The disc-stack centrifuge can be used at low concentration and particle size of cells in a fermentation broth. A disc-stack centrifuge can be employed when the cell and other solid mass includes as little as about 0.2% to about 3% by weight of the fermentation broth. The disc-stack centrifuge can also be used when the cell and other solid mass is less than about 0.2% by weight, for example, 0.01%, 0.05%, and 0.1% by weight, including all values in between. The disc-stack centrifuge can also be used when the cell and other solid mass is more than 3% by weight, for example, 4%, 5%, 6%, 7%, 8%, 9%, 10%, and 15% by weight, including all values in between. When the combined cell mass and other solids is higher than about 3% to about 15% by weight other centrifugation configurations can be used, such as a decanter centrifuge.

Cells and other solid particles that are soft, plastic, and not abrasive, ranging from about 0.5 microns to about 500 microns are generally well-suited for disc-centrifugation. For particulate matter less than about 0.5 microns, ultrafiltration is useful. Likewise, above about 500 microns, a decanter-type centrifuge can be useful. The size of a typical prokaryotic cell that can be cultured to produce a compound of interest, including (R)-3-hydroxybutyl (R)-3-hydroxybutanoate, can range in size from about 0.5 microns to about 10 microns, making disc-stack centrifugation a well-suited method.

Following batch, or during fed-batch or continuous fermentation, cells and insoluble solids can be removed from the fermentation broth by a disc-stack centrifuge. Outputs from a disc-stack centrifuge are a clarified (cell-free) centrate and an underflow stream containing about 5% to about 50% solids. The underflow solids stream from the disc stack centrifuge can contain a significant amount of the product of interest which can be recovered. One way to recover additional compound of interest from the solids is to include further centrifugation steps. In addition to providing greater recovery of the compound of interest, multiple centrifugation also serves to further concentrate the cells and solids. The concentrated cells can be recycled back to the fermentation. Cell recycle is particularly useful when valuable engineered organisms are being used.

In some embodiments, a decanter centrifuge can be employed to separate out the cells and solids. Good performance with a decanter centrifuge is normally realized with solids having particle sizes with a lower limit approaching about 10 microns, although smaller particles can be processed depending on their settling speed as described further below. This centrifuge configuration can be used when the cells of a culture are at the larger size range of a typical prokaryotic organism. One skilled in the art will appreciate that eukaryotic cells are often much larger than prokaryotic cells, with an average eukaryotic cell ranging in size from about 10 microns to about 100 microns or larger. Although a disc-stack centrifuge can operate well in this size range, a decanter centrifuge is useful because it is able to handle larger amounts of solids. Thus, when the cell mass plus other solids is more than about 3 to about 50% of the mass by weight, a decanter centrifuge can be used. This concentration applies to the underflow of the disc stack centrifuge described above, making a decanter centrifuge a well suited method to further concentrate the cell mass and recover additional product.

The decanter, or solid bowl, centrifuge operates on the principle of sedimentation. Exemplary apparatus are described in U.S. Pat Nos. 4,228,949 and 4,240,578, which are incorporated herein by reference in their entirety.

The drum and the screw rotate independently of one another at speeds up to about 3,600 rpm, depending on the type and size of machine. The dewatering principles used are known in the art as the “concurrent” or “counter-current” method. The concurrent method permits very low differential speeds. The differential speed is the difference between the speed of the drum and the speed of the screw. Low differential speeds mean longer residence times in the centrifuge, which result in drier sludge and considerably less wear. The counter-current principle can be more suitable for a feed that is easy to dewater and when a high capacity is desired.

Solids can be separated in solid bowl centrifuges provided their sedimentation speed in the liquid phase portion of the feed is sufficient. Factors that influence sedimentation speed include, for example, particle size, shape, differences in density between the cells/solids and the fermentation broth liquid phase, and viscosity. The geometry of the bowl, especially the relation between the length and diameter, are adaptable to suit the particular conditions. In some embodiments, good results can be obtained at length diameter ratio ranging from about 2:1 to about 3:1.

In operation, separation takes place in a horizontal conical cylindrical bowl equipped with a screw conveyor. The fermentation broth is fed into the bowl through a stationery inlet tube and accelerated by an inlet distributor. Centrifugal force provides the means for sedimentation of the solids on the wall of bowl. A conveyor, rotating in the same direction as bowl with differential speed, conveys the solids to the conical end. The solids are then lifted clear of the liquid phase and centrifugally dewatered before being discharged into a collecting channel. The remaining liquid phase then flows into a housing through an opening in cylindrical end of the bowl.

As described above, the cells and solids can be separated by multiple centrifugation to increase the isolated yield of the compound of interest. Multiple centrifugation can include centrifugation twice, three times, four times, and five times, for example. Intermediate underflow streams can be diluted with water to further increase recovery of the liquid product. Any combination of centrifugation configurations can also be used to perform multiple centrifugations, such as combinations of the disc-stack and decanter centrifugations described above. Further solids that are not separable by centrifugation can be removed through a filtration process, such as ultrafiltration.

Ultrafiltration is a selective separation process through a membrane using pressures up to about 145 psi (10 bar). Useful configurations include cross-flow filtration using spiral-wound, hollow fiber, or flat sheet (cartridge) ultrafiltration elements. These elements consist of polymeric or ceramic membranes with a molecular weight cut-off of less than about 200,000 Daltons. Ceramic ultrafiltration membranes are also useful since they have long operating lifetimes of up to or over 10 years. Ceramics have the disadvantage of being much more expensive than polymeric membranes. Ultrafiltration concentrates suspended solids and solutes of molecular weight greater than about 1,000 Daltons. Ultrafiltration includes filtering through a membrane having nominal molecular weight cut-offs (MWCO) from about 1,000 Daltons to about 200,000 Daltons (pore sizes of about 0.005 to 0.1 microns). For example, ultrafiltration membranes can have pore sizes from about 0.005 microns to 0.1 micron, or from about 0.005 microns to 0.05 microns, about 0.005 microns to 0.02 micron, or about 0.005 microns to 0.01 microns. For example, ultrafiltration membranes can have a MWCO from about 1,000 Daltons to 200,000 Daltons, about 1,000 Daltons to 50,000 Daltons, about 1,000 Daltons to 20,000 Daltons, about 1,000 Daltons to 5,000 Daltons, or with about 5,000 Daltons to 50,000 Daltons. Using ultrafiltration the permeate liquid will contain low-molecular-weight organic solutes, such as (R)-3-hydroxybutyl (R)-3-hydroxybutanoate or target compound, media salts, and water. The captured solids can include, for example, residual cell debris, DNA, and proteins. Diafiltration techniques well known in the art can be used to increase the recovery of (R)-3-hydroxybutyl (R)-3-hydroxybutanoate or target compound in the ultrafiltration step.

In addition to the use ultrafiltration downstream of centrifugation, ultrafiltration can also be used downstream of microfiltration. Microfiltration, for example, involves a low-pressure membrane process for separating colloidal and suspended particles in the range of about 0.05-10 microns. Useful configurations include cross-flow filtration using spiral-wound, hollow fiber, or flat sheet (cartridge) microfiltration elements. Microfiltration includes filtering through a membrane having pore sizes from about 0.05 microns to about 10.0 microns. Microfiltration membranes can have nominal molecular weight cut-offs (MWCO) of about 20,000 Daltons and higher. The term molecular weight cut-off is used to denote the size of particle, including polypeptides, or aggregates of peptides, that will be approximately 90% retained by the membrane. Polymeric, ceramic, or steel microfiltration membranes can be used to separate cells. Ceramic or steel microfiltration membranes have long operating lifetimes including up to or over 10 years. Microfiltration can be used in the clarification of fermentation broth. For example, microfiltration membranes can have pore sizes from about 0.05 microns to 10 micron, or from about 0.05 microns to 2 microns, about 0.05 microns to 1.0 micron, about 0.05 microns to 0.5 microns, about 0.05 microns to 0.2 microns, about 1.0 micron to 10 microns, or about 1.0 micron to 5.0 microns, or membranes can have a pore size of about 0.05 microns, about 0.1 microns, or about 0.2 microns For example, microfiltration membranes can have a MWCO from about 20,000 Daltons to 500,000 Daltons, about 20,000 Daltons to 200,000 Daltons, about 20,000 Daltons to 100,000 Daltons, about 20,000 Daltons to 50,000 Daltons, or with about 50,000 Daltons to 300,000 Daltons; or with a MWCO of about 20,000 Daltons, about 50,000 Dalton, about 100,000 Daltons or about 300,000 Daltons can be used in separating cell and solids from the fermentation broth.

A further filtration procedure called nanofiltration can be used to separate out certain materials by size and charge, including carbohydrates, inorganic and organic salts, residual proteins and other high molecular weight impurities that remain after the previous filtration step. This procedure can allow the recovery of certain salts without prior evaporation of water, for example. Nanofiltration can separate salts, remove color, and provide desalination. In nanofiltration, the permeate liquid generally contains monovalent ions and low-molecular-weight organic compounds as exemplified by (R)-3-hydroxybutyl (R)-3-hydroxybutanoate or target compound. Nanofiltration includes filtering through a membrane having nominal molecular weight cut-offs (MWCO) from about 100 Daltons to about 2,000 Daltons (pore sizes of about 0.0005 to 0.005 microns). For example, nanofiltration membranes can have a MWCO from about 100 Daltons to 500 Daltons, about 100 Daltons to 300 Daltons, or about 150 Daltons to 250 Daltons. The mass transfer mechanism in nanofiltration is diffusion. The nanofiltration membrane allows the partial diffusion of certain ionic solutes (such as sodium and chloride), predominantly monovalent ions, as well as water. Larger ionic species, including divalent and multivalent ions, and more complex molecules are substantially retained (rejected). Larger nonionic species, such as carbohydrates are also substantially retained (rejected). Nanofiltration is generally operated at pressures from 70 psi to 700 psi, from 200 psi to 650 psi, from 200 psi to 600 psi, from 200 psi to 450 psi, from 70 psi to 400 psi, of about 400 psi, of about 450 psi or of about 500 psi.

Since monovalent ions are partially diffusing through the nanofiltration membrane along with the water, the osmotic pressure difference between the solutions on each side of the membrane is not as great and this typically results in somewhat lower operating pressure with nanofiltration compared with, for example, reverse osmosis.

Nanofiltration not only removes a portion of the inorganic salts but can also remove salts of organic acids. The removal of organic acid byproducts can be important in the isolation process because such acids can catalyze or serve as a reactant in undesirable side reactions with a product of interest. In the context of specific embodiments related to the isolation of (R)-3-hydroxybutyl (R)-3-hydroxybutanoate, for example, the removal of organic acids is particularly useful because it can prevent reactions such as esterification of the hydroxyl groups during the elevated temperatures of any downstream evaporation or distillation steps. These ester byproducts may have higher boiling points than (R)-3-hydroxybutyl (R)-3-hydroxybutanoate resulting in yield losses to the heavies stream in distillation.

Nanofiltration can also separate the glucose or sucrose substrate from the product of interest, preventing degradation reactions during evaporation and distillation. These degradation reactions can produce coloration of the compound of interest. The salt and substrate rich nanofiltration retentate can be better suited for recycle to fermentation compared to a recovered salt stream from evaporative crystallization. For example, the use of filtration methods in lieu of methods involving application of heat can result in fewer degradation products. Such degradation products can be toxic to the fermentation organism.

Both nanofiltration and ion exchange can remove color forming compounds and UV absorbing compounds. This can be useful in the context of some compounds of interest.

Multiple filtration membranes can be used serially with gradually increasing refinement of the size of the solids that are retained. This can be useful to reduce fouling of membranes and aid in recovering individual components of the fermentation broth for recycle. For example, a series of filtrations can utilize microfiltration, followed by ultrafiltration, followed by nanofiltration. Thus, microfiltration aids in recovery of cell mass, ultrafiltration removes large components such as cell debris, DNA, and proteins, and nanofiltration aids in recovery of salts.

Those skilled in the art will recognize that any of the various filtration types can be integrated within the context of a variety of fermentation bioreactor configurations given the teachings and guidance provide herein. In some embodiments the filtration occurs external to the bioreactor. In this mode, any amount of the fermentation broth can be removed from the bioreactor and filtered separately. Filtration can be aided by use of vacuum methods, or the use of positive pressure. In some embodiments, cell filtration can be accomplished by means of a filtration element internal to the bioreactor. Such configurations include those found in membrane cell-recycle bioreactors (MCRBs). Chang et al. U.S. Pat. No. 6,596,521 have described a two-stage cell-recycle continuous reactor.

In some embodiments, the cells can be separated and recycled into the fermentation mixture by means of an acoustic cell settler as described by Yang et al. (Biotechnol. Bioprocess. Eng., 7:357-361(2002)). Acoustic cell settling utilizes ultrasound to concentrate the suspension of cells in a fermentation broth. This method allows for facile return of the cells to the bioreactor and avoids the issue of membrane fouling that sometimes complicates filtration-type cell recycle systems.

With respect to isolation of salts prior to water evaporation, other methods can be used alone, or in combination with the above exemplary filtration processes. Such other methods include, for example, ion exchange. For example, Gong et al. (Desalination 191:1-3, 193-199 (2006)) have described the effects of transport properties of ion-exchange membranes on desalination of 1,3-propanediol fermentation broth by electrodialysis.

Ion exchange can be used to remove salts from a mixture, such as for example, a fermentation broth. Ion exchange elements can take the form of resin beads as well as membranes. Frequently, the resins can be cast in the form of porous beads. The resins can be cross-linked polymers having active groups in the form of electrically charged sites. At these sites, ions of opposite charge are attracted, but can be replaced by other ions depending on their relative concentrations and affinities for the sites. Ion exchange resins can be cationic or anionic, for example. Factors that determine the efficiency of a given ion exchange resin include the favorability for a given ion, and the number of active sites available. To maximize the active sites, large surface areas can be useful. Thus, small porous particles are useful because of their large surface area per unit volume.

The anion exchange resins can be strongly basic or weakly basic anion exchange resins, and the cation exchange resin can be strongly acidic or weakly acidic cation exchange resin. Non-limiting examples of ion-exchange resin that are strongly acidic cation exchange resins include AMBERJET™ 1000 Na, AMBERLITE™ IR10 or DOWEX™ 88; weakly acidic cation exchange resins include AMBERLITE™ IRC86 or DOWEX™ MAC3; strongly basic anion exchange resins include AMBERJET™ 4200 Cl or DOWEX™ 22; and weakly basic anion exchange resins include AMBERLITE™ IRA96, DOWEX™ 66 or DOWEX™ Marathon WMA. Ion exchange resins can be obtained from a variety of manufacturers such as Dow, Purolite, Rohm and Haas, Mitsubishi or others.

A primary ion exchange can be utilized for the removal of salts. The primary ion exchange can include, for example, both a cation exchange or an anion exchange, or a mixed cation-anion exchange, which include both cation exchange and anion exchange resins. In certain embodiments, primary ion exchange can be cation exchange and anion exchange in any order. In some embodiments, the primary ion exchange is an anion exchange followed by a cation exchange, or a cation exchange followed by an anion exchange, or a mixed cation-anion exchange. In certain embodiments, the primary ion exchange is an anion exchange, or a cation exchange. More than one ion exchange of a given type, can be used in the primary ion exchange. For example, the primary ion exchange can include a cation exchange, followed by an anion exchange, followed by a cation exchange and finally followed by an anion exchange.

In certain embodiments, the primary ion exchange uses a strongly acidic cation exchange and a weakly basic anion exchange Ion exchange, for example, primary ion exchange, can be carried out at temperatures from 20° C. to 60° C., from 30° C. to 60° C., 30° C. to 50° C., 30° C. to 40° C. or 40° C. to 50° C.; or at about 30° C., about 40° C., about 50° C., or about 60° C. Flow rates in ion exchange, such as primary ion exchange, can be from 1 bed volume per hour (BV/h) to 10 BV/h, 2 BV/h to 8 BV/h, 2 BV/h to 6 BV/h, 2 BV/h to 4 BV/h, 4 BV/h to 6 BV/h, 4 BV/h to 8 BV/h, 4 BV/h to 10 BV/h or 6 BV/h to 10 BV/h.A useful aspect of ion exchange is the facility with which the resin can be regenerated. The resin can be flushed free of the exchanged ions and contacted with a solution of desirable ions to replace them. With regeneration, the same resin beads can be used over and over again, and the isolated ions can be concentrated in a waste effluent. As with the many filtration methods, serial ion exchange can be performed. Thus, a feed can be passed through both any number of anionic and cationic exchangers, or mixed-bed exchangers, and in any order.

When salts are removed by nanofiltration and/or ion exchange, a reverse osmosis (RO) membrane filtration can be used to remove a portion of the water prior to evaporation. Water permeates the RO membrane while (R)-3-hydroxybutyl (R)-3-hydroxybutanoate or target compound is retained. In some embodiments, an RO membrane can concentrate a product, such as (R)-3-hydroxybutyl (R)-3-hydroxybutanoate or target compound to about 20%. One skilled in the art will recognize that the osmotic pressure from the (R)-3-hydroxybutyl (R)-3-hydroxybutanoate or target compound increases to a point where further concentration using an RO membrane can no longer be viable. Nonetheless, the use of an RO membrane is a useful low energy input method for concentrating (R)-3-hydroxybutyl (R)-3-hydroxybutanoate or target compound prior to the more energy intensive water evaporation process. Thus, on large scale, employing a RO membrane can be particularly useful.

Polishing is a procedure to remove any remaining salts and/or other impurities in a crude (R)-3-hydroxybutyl (R)-3-hydroxybutanoate or target compound mixture. The polishing can include contacting the crude (R)-3-hydroxybutyl (R)-3-hydroxybutanoate or target compound mixture with a number of materials that can react with or adsorb the impurities in the crude (R)-3-hydroxybutyl (R)-3-hydroxybutanoate or target compound mixture. The materials used in the polishing can include ion exchange resins, activated carbon, or adsorbent resins, such as, for example, DOWEX™ 22, DOWEX™ 88, OPTIPORE™ L493, AMBERLITE™ XAD761 or AMBERLITE™ FPX66, or mixtures of these resins, such as a mixture of DOWEX™ 22 and DOWEX™ 88.

In one embodiment, the polishing is a polishing ion exchange. The polishing ion exchange can be used to remove any residual salts, color bodies and color precursors before further purification. The polishing ion exchange can include an anion exchange, a cation exchange, both a cation exchange and anion exchange, or can be a mixed cation-anion exchange, which includes both cation exchange and anion exchange resins. In certain embodiments, the polishing ion exchange is an anion exchange followed by a cation exchange, a cation exchange followed by an anion exchange, or a mixed cation-anion exchange. In certain embodiment, the polishing ion exchange is an anion exchange. The polishing ion exchange includes both strong cation and strong anion exchange, or includes strong anion exchange without other polishing cation exchange or polishing anion exchange. The polishing ion exchange occurs after a water removal step such as evaporation, and prior to a subsequent distillation.

In some embodiments, water removal via evaporation is used to facilitate salt recovery. In some embodiments, the salts have been removed prior to water removal. In either case, evaporated water can be recycled as makeup water to the fermentation, minimizing the overall water requirements for the process. In the case where the salts have not been removed, their solubility in the (R)-3-hydroxybutyl (R)-3-hydroxybutanoate enriched liquid phase is sufficiently low that they can crystallize after water removal. In some embodiments the salts have a sufficiently low solubility in (R)-3-hydroxybutyl (R)-3-hydroxybutanoate that the separated (R)-3-hydroxybutyl (R)-3-hydroxybutanoate is about 98% salt-free.

An evaporative crystallizer can be used to generate precipitated salts which can be removed by centrifugation, filtration or other mechanical means. In the context of (R)-3-hydroxybutyl (R)-3-hydroxybutanoate isolation, an evaporative crystallizer serves to remove water from the fermentation broth creating a liquid phase that has removed enough water to cause supersaturation of the fermentation media salts and subsequent crystallization in the remaining liquid phase or mother liquor.

The mother liquor refers to the bulk solvent in a crystallization. Frequently, the mother liquor is a combination of solvents with different capacity to solublize or dissolve various solutes. In the context of the purification of (R)-3-hydroxybutyl (R)-3-hydroxybutanoate from a fermentation broth, for example, the mother liquor includes the liquid fraction obtained after removing cells and other solids from the fermentation broth. In the context of isolating a compound of interest from a fermentation broth, the primary solute includes the fermentation media salts and organic acids.

Supersaturation in crystallization refers to a condition in which a solute is more concentrated in a bulk solvent than is normally possible under given conditions of temperature and pressure. The bulk solvent of the fermentation broth is water containing relatively smaller amounts of (R)-3-hydroxybutyl (R)-3-hydroxybutanoate, for example, and dissolved salts and other media.

A forced circulation (FC) crystallizer has been described, for example, in U.S. Pat. No. 3,976,430 which is incorporated by reference herein in its entirety. The FC crystallizer evaporates water resulting in an increased supersaturation of the salts in the compound-enriched (such as (R)-3-hydroxybutyl (R)-3-hydroxybutanoate) liquid fraction thus causing the salts to crystallize. The FC crystallizer is useful for achieving high evaporation rates. The FC crystallizer consists of four basic components: a crystallizer vessel with a conical bottom portion, a circulating pump, a heat exchanger, and vacuum equipment which handles the vapors generated in the crystallizer. Slurry from the crystallizer vessel is circulated through the heat exchanger, and returned to the crystallizer vessel again, where supersaturation is relieved by deposition of salts on the crystals present in the slurry. The evaporated water is conducted to the vacuum system, where it is condensed and recycled to the fermentation broth as desired. Although in some embodiments, there is a low vacuum, it is also possible to use the FC crystallizer at about atmospheric pressure as well. In some embodiments, the FC crystallizer utilizes adiabatic evaporative cooling to generate salt supersaturation. In such embodiments, the FC crystallizer need not be equipped with a heat exchanger.

In some embodiments, the FC crystallizer can be further equipped with internal baffles to handle overflow of the liquid phase and to reduce fines which can inhibit crystal growth. The salts generated in the FC crystallizer can also be size selected with the aid of an optional elutriation leg. This portion of the FC crystallizer appears at the bottom of the conical section of the crystallizer vessel. Size selection is achieved by providing a flow of fermentation fluid up the leg allowing only particles with a particular settling rate to move against this flow. The settling speed is related to the size and shape of the crystals as well as fluid viscosity. In further embodiments, the FC crystallizer can also be equipped with an internal scrubber to reduce product losses. This can assist in the recovery of volatile products.

The turbulence or draft tube and baffle “DTB” crystallizer provides two discharge streams, one of a slurry that contains crystals, and another that is the liquid phase with a small amount of fines. The configuration of the DTB crystallizer is such that it promotes crystal growth, and can generate crystals of a larger average size than those obtained with the FC crystallizer. In some embodiments, the DTB crystallizer operates under vacuum, or at slight superatmospheric pressure. In some embodiments, the DTB crystallizer uses vacuum for cooling.

In some embodiments, a DTB crystallizer operates at a low supersaturation. One skilled in the art will appreciate that large crystals can be obtained under this regime. The system can be optionally configured to dissolve fines to further increase crystal size. When the DTB crystallizer is used in fermentation media salt recovery, crystal size is not necessarily a priority.

The DTB crystallizer has been studied widely in crystallization, and can be modeled with accuracy. Its distinct zones of growth and clarified liquid phase facilitate defining kinetic parameters, and thus, the growth and nucleation rate can be readily calculated. These features make the DTB crystallizer suitable to mathematical description, and thus, subject to good operating control. The DTB crystallizer is an example of a mixed suspension mixed product removal (MSMPR) design, like the FC crystallizer.

The DTB crystallizer includes a baffled area, serving as a settling zone, which is peripheral to the active volume. This zone is used to further process the liquid phase and fines. In some embodiments, the baffled area is not present, as can be the case where further processing of fines is less important. Such a configuration is known in the art as a draft-tube crystallizer. A DTB crystallizer can be equipped with an agitator, usually at the bottom of the apparatus in the vicinity of the entry of the feed solution. Like the FC crystallizer, the DTB crystallizer is optionally equipped with an elutriation leg. In some embodiments, an optional external heating loop can be used to increase evaporation rates.

Yet another crystallizer configuration is the induced circulation crystallizer. This configuration provides additional agitation means for the active volume. The apparatus is similar to the DTB crystallizer with respect to the use of a draft tube. Unlike the DTB apparatus, there is no internal agitator. Instead, an inducer in the conical portion of the vessel introduces heated solution from a recirculation pump. As with other crystallization apparatus configurations, the induced circulation crystallizer is optionally equipped with an elutriation leg. Baffles can also be optionally employed with this type of crystallizer.

In still further embodiments, the crystallizer can be an Oslo-type crystallizer. This type of crystallizer is also referred to as “growth- ”, “fluid-bed-”, or “Krystal-” type crystallizer. The Oslo crystallizer allows the growth of crystals in a fluidized bed, which is not subject to mechanical circulation. A crystal in an Oslo unit will grow to a size proportional to its residence time in the fluid bed. The result is that an Oslo crystallizer can grow crystals larger than most other crystallizer types. The slurry can be removed from the crystallizer’s fluidized bed and sent to, for example, a centrifugation section. Clear liquid phase containing (R)-3-hydroxybutyl (R)-3-hydroxybutanoate can be purged from the crystallizer’s clarification zone.

The classifying crystallization chamber is the lower part of the unit. The upper part is the liquor-vapor separation area where supersaturation is developed by the removal of water. The slightly supersaturated liquid phase flows down through a central pipe and the supersaturation is relieved by contact with the fluidized bed of crystals. The desupersaturation occurs progressively as the circulating liquid phase moves upwards through the classifying bed before being collected in the top part of the chamber. The remaining liquid leaves via a circulating pipe and after addition of the fresh feed, it passes through the heat exchanger where heat make-up is provided. It is then recycled to the upper part.

In some embodiments, the Oslo type crystallizer can also be optionally equipped with baffles, an elutriation leg, and scrubber as described above. Since the growing crystals are not in contact with any agitation device, the amount of fines to be destroyed is generally lower. The Oslo type crystallizer allows long cycles of production between periods for crystal removal.

The Oslo-type crystallizer is useful for the separation-crystallization of several chemical species as would be found in fermentation media salts. In one embodiment, the Oslo type crystallization unit is of the “closed” type. In other embodiments the Oslo-type crystallizer is the “open” type. The latter configuration is useful when large settling areas are needed, for example.

Many of the foregoing evaporative crystallization apparatus allow for controlled crystal growth. In the recovery of fermentation media salts from the liquid portion after cell removal, the exact crystal morphology, size, and the like are generally inconsequential. Indeed, recovery of amorphous media salts can be sufficient in the purification of any compound of interest, including (R)-3-hydroxybutyl (R)-3-hydroxybutanoate. Thus, in some embodiments, other evaporation methods can be utilized that do not control crystal growth per se.

When salts are removed by nanofiltration and/or ion exchange, a reverse osmosis (RO) membrane filtration can be used to remove a portion of the water prior to evaporation. Water permeates the RO membrane while (R)-3-hydroxybutyl (R)-3-hydroxybutanoate is retained. In some embodiments, an RO membrane can concentrate a product, such as (R)-3-hydroxybutyl (R)-3-hydroxybutanoate to about 20%. One skilled in the art will recognize that the osmotic pressure from the product (R)-3-hydroxybutyl (R)-3-hydroxybutanoate increases to a point where further concentration using an RO membrane is no longer viable. Nonetheless, the use of an RO membrane is a useful low energy input method for concentrating the product of interest prior to the more energy intensive water evaporation process. Thus, on large scale, employing a RO membrane is particularly useful.

In some embodiments, substantially all of the salts are removed prior to removal of water. In other embodiments, substantially all of the salts are removed after removal of a portion of water. The portion of water removed can be any amount including 5%, 10%, 15%, 20%, 25%, 30%, 35%, 40%, 50%, 60%, 70%, 80%, and all values in between. In some embodiments, salts are removed after removal of substantially all of the water. Substantially all the water includes 95%, 96%, 97%, 98%, 99%, 99.9% and all values in between and including all the water.

There are many types and configurations of evaporators available for water removal. One consideration for designing an evaporation system is minimizing energy requirements. Evaporation configurations such as multiple effects or mechanical vapor recompression allow for reduced energy consumption. In some embodiments, removing water is accomplished by evaporation with an evaporator system which includes one or more effects. In some embodiments, a double- or triple-effect evaporator system can be used to separate water from a product of interest, such as (R)-3-hydroxybutyl (R)-3-hydroxybutanoate. Any number of multiple-effect evaporator systems can be used in the removal of water. These apparatus can also be applied to any fermentation product that having a boiling point higher than water. A triple effect evaporator, or other evaporative apparatus configuration, can include dedicated effects that are evaporative crystallizers for salt recovery, for example the final effect of a triple effect configuration.

An evaporator is a heat exchanger in which a liquid is boiled to give a vapor that is also a low pressure steam generator. This steam can be used for further heating in another evaporator called another “effect.” Thus, for example, two evaporators can be connected so that the vapor line from one is connected to the steam chest of the other providing a two, or double-effect evaporator. This configuration can be propagated to a third evaporator to create a triple-effect evaporator, for example.

Evaporators can therefore be classified by the number of effects. In a single-effect evaporator, steam provides energy for vaporization and the vapor product is condensed and removed from the system. In a double-effect evaporator, the vapor product off the first effect is used to provide energy for a second vaporization unit. The cascading of effects can continue for any number of stages. Multiple-effect evaporators can remove large amounts of solvent more efficiently relative to a single effect evaporator.

In a multiple effect arrangement, the latent heat of the vapor product off of an effect is used to heat the following effect. Effects are numbered beginning with the one heated by steam, Effect I. The first effect operates under the highest pressure. Vapor from Effect I is used to heat Effect II, which consequently operates at lower pressure. This continues through each addition effect, so that pressure drops through the sequence and the hot vapor will travel from one effect to the next.

In some embodiments, all effects in an evaporator can be physically similar in size, construction, and heat transfer area. Unless thermal losses are significant, they can also have the same capacity as well. Evaporator trains, the serially connected effects, can receive feed in several different ways. Forward Feed arrangements follow the pattern I, II, and III. These use a single feed pump. In this configuration the feed is raised to the highest operating temperature as used in Effect I. The lowest operating temperature is in the final effect, where the product is also most concentrated. Therefore, this configuration is useful for products that are heat sensitive or to reduce side reactions.

In other embodiments, Backward Feed arrangements, III, II, I can be used. In such a configuration multiple pumps are used to work against the pressure drop of the system, however, since the feed is gradually heated they can be more efficient than a forward feed configuration. This arrangement also reduces the viscosity differences through the system and is thus useful for viscous fermentation broths. In some embodiments, Mixed Feed arrangements can be utilized, with the feed entering in the middle of the system, or effects II, III, and I. The final evaporation is performed at the highest temperature. Additionally, fewer pumps are required than in a backward feed arrangement. In still further embodiments, a Parallel Feed system is used to split the feed stream and feed a portion to each effect. This configuration is common in crystallizing evaporators where the product is expected to be a slurry.

There are numerous evaporator designs. Any combination of designs can be used as an effect as described above. One evaporator design is the falling film evaporator. This apparatus includes a vertical shell-and-tube heat exchanger, with a laterally or concentrically arranged centrifugal separator.

The liquid to be evaporated is evenly distributed on the inner surface of a tube. The liquid flows downwards forming a thin film, from which evaporation takes place because of the heat applied by the steam. The steam condenses and flows downwards on the outer surface of the tube. A number of tubes are built together side by side. At each end the tubes are fixed to tube plates, and finally the tube bundle is enclosed by a jacket.

The steam is introduced through the jacket. The space between the tubes forms the heating section. The inner side of the tubes is called the boiling section. Together they form the calandria. The concentrated liquid and the vapor leave the calandria at the bottom part, from where the main proportion of the concentrated liquid is discharged. The remaining part enters the subsequent separator tangentially together with the vapor. The separated concentrate is discharged, usually be means of the same pump as for the major part of the concentrate from the calandria, and the vapor leaves the separator from the top. The heating steam, which condenses on the outer surface of the tubes, is collected as condensate at the bottom part of the heating section, from where it is discharged.

Falling film evaporators can be operated with very low temperature differences between the heating media and the boiling liquid, and they also have very short product contact times, typically just a few seconds per pass. These characteristics make the falling film evaporator particularly suitable for heat-sensitive products. Operation of falling film evaporators with small temperature differences facilitates their use in multiple effect configurations or in conjunction with mechanical vapor compression systems.

Sufficient wetting of the heating surface in tubes of the calandria helps avoid dry patches and incrustations which can clog the tubes. In some embodiments, the wetting rate can be increased by extending or dividing the evaporator effects. Falling film evaporators are highly responsive to alterations of parameters such as energy supply, vacuum, feed rate, and concentrations, for example. In some embodiments, a single, double, triple, or other multiple-effect falling film evaporator configuration can utilize fermentation feed that has been filtered through a nanofiltration process as detailed above. Reducing the salts prior to water evaporation can further help prevent incrustation in the tubes of the calandria.

In some embodiments, the falling film evaporator is a short path evaporator. In operation the liquid fraction is evenly distributed over the heating tubes of the calandria by means of a distribution system. The liquid fraction flows down in a thin film on the inside walls in a manner similar to the conventional falling film evaporator. The vapors formed in the in the calandria tubes are condensed as a distillate on external walls of condensate tubes and then flows downward. Water distillate and the enriched liquid fraction are separately discharged from the lower part of the evaporator.

Another evaporator configuration is the forced circulation evaporator. In this design a flash vessel or separator is disposed above a calandria and circulation pump. In operation, the liquid fraction is circulated through the calandria by means of a circulation pump. The liquid is superheated within the calandria at an elevated pressure higher than the normal boiling pressure. Upon entering the separator, the pressure is rapidly reduced resulting in flashing or rapid boiling of the liquid. The flow velocity, controlled by the circulation pump, and temperatures can be used to control the water removal process. This configuration is useful for avoiding fouling of the calandria tubes.

In some embodiments, multiple forced circulation evaporator effects can be used as described above. For example, in addition to a single effect forced circulation evaporator, double, triple, and multiple effect forced circulation evaporators can be used in the separation of water from the liquid fraction of the fermentation liquid. In some embodiments, one or more forced circulation evaporators can be used in conjunction with one or more falling film evaporators.

In still further embodiments, the evaporator can be a plate evaporator. This evaporator uses a plate heat exchanger and one or more separators. A plate-and-frame configuration uses plates with alternating channels to carry heating media and the liquid fraction of the fermentation broth. In operation, the liquid phase and heating media are passed through their respective channels in counterflow. Defined plate distances and shapes generate turbulence resulting in efficient heat transfer. The heat transfer to the channels with the liquid fraction causes water to boil. The vapor thus formed drives the residual liquid as a rising film into a vapor duct of the plate assembly. Residual liquid and vapors are separated in the downstream centrifugal separator. The wide inlet duct and the upward movement assist in good distribution over the cross-section of the heat exchanger. A plate evaporator can be usefully operated with a pre-filtration through a nanofiltration membrane to avoid fouling. Thus, similar considerations as the falling film evaporator with respect to incrustation are warranted.

In some embodiments, multiple-effect plate evaporation can be utilized in much the same manner as described above for falling film and forced circulation evaporators. When used in multiple effect configurations, one skilled in the art will recognize the benefit of using a forced circulation evaporator and/or a nanofiltration step prior to introduction of the liquid fraction to a plate evaporator. Thus, a separation scheme can include, for example, nanofiltration, followed by a multiple-effect evaporation configuration of one or more forced circulation evaporators, followed by one or more of a plate and/or falling film evaporator. In still further embodiments, any of the evaporative crystallizers described above can also be used in conjunction with a multiple-effect configuration.

In some embodiments, a circulation evaporator can be used to remove water from the liquid fraction. The circulation evaporator utilizes a vertical calandria with short tube length with a lateral separator disposed at the top of the heat exchanger. In operation the liquid fraction is supplied at the bottom of the calandria and rises to the top. During heating in the tubes of the calandria, the water begins to boil releasing vapor. The liquid is carried to the top of the calandria entrained by the upward moving vapors. The liquid is separated from the vapors as it enters the separator. The liquid flows back into the evaporator via a circulation pipe to allow continued circulation. The larger the temperature difference between the heating elements of the calandria and the separator chamber results in larger degree of water evaporation from the liquid fraction. When the liquid portion is sufficiently enriched in (R)-3-hydroxybutyl (R)-3-hydroxybutanoate, the salts will begin to precipitate from the liquid fraction.

In some embodiments, the separator of the circulation evaporator can be partitioned into several separation chambers each equipped with its own liquid circulation system. This can reduce the heating surface needed to remove water from the liquid fraction.

The fluidized bed evaporator is yet another configuration that can be used for water removal from the liquid fraction. Such a system is equipped with a vertical fluidized bed heat exchanger. On the tube side of the heat exchanger are solid particles such as glass or ceramic beads, or steel wire particles.

The fluidized bed evaporator operates in a similar manner to the forced circulation evaporator. The upward movement of the liquid entrains the solid particles which provides a scouring or cleaning action. Together with the liquid fraction they are transferred through the calandria tubes. At the head of the calandria, the solid particles are separated from the liquid and are recycled to the calandria inlet chamber. The superheated fluid is flashed to boiling temperature in the separator allowing removal of water through evaporation. The scouring action of the solids in the tubes of the calandria allow for prolonged operation times and further retard fouling of the tubes. This can be useful when the creation of fouling solids limits the use of conventional forced circulation evaporator systems.

The rising film evaporator is yet another type of evaporator useful in the removal of water from the liquid fraction collected from the fermentation broth. This system configuration has a top-mounted vapor separator on a vertical shell-and-tube heat exchanger (calandria). In operation, the liquid fraction at the bottom of the calandria rises to the top to the vapor separator. External heating causes the water in the liquid fraction to boil in the inside walls of the calandria tubes. The upward movement of the steam causes the liquid fraction to be carried to the top of the calandria. During ascent though the tube further vapor is formed. Upon entry into the separator vapors and liquid phases are separated. The rising film evaporator is particularly useful when used with viscous liquids and/or when large amounts of fouling solids are expected.

The counterflow-trickle evaporator is yet another evaporator that can be used for water removal from the liquid fraction of the fermentation broth. This apparatus has a shell-and-tube heat exchanger (calandria) with the lower part of the calandria larger than that of a rising film evaporator. Disposed on top of the calandria, like the rising film evaporator is a separator. In this evaporator the separator is further equipped with a liquid distribution system.

In operation, liquid is provided at the top of the evaporator like a falling film evaporator. The liquid is distributed over the evaporator tubes, but vapor flows to the top in counterflow to the liquid. In some embodiments, the process can also include a stream of an inert gas, for example, to enhance entrainment. This gas can be introduced in the lower portion of the calandria.

A stirrer evaporator is yet another type of evaporator that can be used for water removal from the liquid fraction of the fermentation broth. This apparatus includes an external, jacket-heated vessel equipped with a stirrer. In operation, the liquid fraction is placed in the vessel, optionally in batches. The water is evaporated off by boiling with continuous stirring to a desired concentration. This apparatus can increase its evaporation rate by increasing the heating surface by use of optional immersion heating coils. This type of evaporator is particularly useful when the fermentation is highly viscous.

Finally, the spiral tube evaporator is another type of evaporator that can be used for water removal from the liquid fraction of the fermentation broth. The design includes a heat exchanger equipped with spiral heating tubes and a bottom-mounted centrifugal separator. In operation, the liquid fraction flows a boiling film from top to bottom in parallel flow to the vapor. The expanding vapors produce a shear, or pushing effect on the liquid film. The curvature of the path of flow induces a secondary flow which interferes with the movement along the tube axis. This turbulence improves heat transfer and is particularly useful with viscous liquids. The spiral configuration of the heating tubes usefully provides a large heating surface area to height ratio relative to a non-spiral, straight tube design. This apparatus provides large evaporation ratios allowing single pass operation.

As described above, the use of multiple evaporators of any type described above in double, triple, and multi-effect configurations can increase the efficiency of evaporation. Other methods to improve efficiency of operation include, for example, thermal and mechanical vapor recompression. In some embodiments, any combination of multiple-effect configurations, thermal recompression, and mechanical recompression can be used to increase evaporation efficiency.

Thermal vapor recompression involves recompressing the vapor from a boiling chamber (or separator) to a higher pressure. The saturated steam temperature corresponding to the heating chamber pressure is higher so that vapor can be reused for heating. This is accomplished with a steam jet vapor recompressor which operates on the steam jet pump principle. Briefly, the steam jet principle utilizes the energy of steam to create vacuum and handle process gases. Steam under pressure enters a nozzle and produces a high velocity jet. This jet action creates a vacuum that draws in and entrains gas. The mixture of steam and gas is discharged at atmospheric pressure. A quantity of steam, called motive steam, is used to operate the thermal recompressor. The motive steam is transferred to the next effect or to a condenser. The energy of the excess vapor is approximately that of the motive steam quantity used.

In multiple-effect evaporators equipped with thermal vapor recompressors, the heating medium in the first calandria is the product vapor from one of the associated effects, compressed to a higher temperature level by means of a steam ejector. The heating medium in any subsequent effect is the vapor generated in the previous calandria. Vapor from the final effect is condensed with incoming product, optionally supplemented by cooling water as necessary. All recovered water is readily recycled to a fermentation broth.

Mechanical recompressors utilize all vapor leaving one evaporator. The vapor is recompressed to the pressure of the corresponding heating steam temperature of the evaporator. The operating principle is similar to a heat pump. The energy of the vapor condensate can be optionally used to pre-heat further portions of the liquid fraction of the fermentation broth. The mechanical recompression is supplied by use of a high pressure fans or turbocompressors. These fans operate a high velocity and are suited for large flow rates at vapor compression ratios of about 1:1.2 to about 1:2. Rational speeds can be between about 3,000 to about 18,000 rpm. In some embodiments, when particularly high pressures are useful, multiple stage compressors can be used.

In evaporators with equipped with mechanical vapor recompressors, the heating medium in the first effect is vapor developed in the same effect, compressed to a higher temperature by means of a high-pressure fan. Any excess vapor from the high heat section is optionally condensed or can be utilized in a high concentrator.

As described above there are many possible evaporation types that can be arranged in various energy efficient configurations including multiple effect, thermal vapor recompression, mechanical vapor recompression, or combinations of these. Optimal configurations depend on many factors, including, for example, whether media salts are removed prior to evaporation or via crystallization during the evaporation. For the case where salts are removed prior to evaporation, low cost configurations are useful. Exemplary configurations include a falling film triple effect evaporator system or mechanical vapor recompression system. The case where salts are crystallized during the evaporation is more complex due to the possibility of scaling of the heat exchanger surfaces by precipitation of the salts. An exemplary configuration for this case includes triple effect where the first two effects are falling film evaporators (before the onset of crystallization) and the final stage is a forced circulation evaporative crystallizer, for example.

(R)-3-hydroxybutyl (R)-3-hydroxybutanoate purification, in particular, can occur in a series of two distillation columns, although more can be used. A first column is used to separate water and other light components from (R)-3-hydroxybutyl (R)-3-hydroxybutanoate, while a second column is used to distill the (R)-3-hydroxybutyl (R)-3-hydroxybutanoate from any residual heavy components. The distillation columns can be operated under vacuum to reduce the required temperatures and reduce unwanted reactions, product degradation, and color formation. Pressure drop across the columns can be minimized to maintain low temperatures in the bottom reboiler. Residence time in the reboiler can be minimized to also prevent unwanted reactions, product degradation, and color formation, by using, for example, a falling film reboiler.

Those skilled in the art will recognize that various configurations of the enumerated centrifugation, filtration, ion exchange, evaporator crystallizer, evaporator, and distillation apparatus are useful in the purification of a compound of interest, including (R)-3-hydroxybutyl (R)-3-hydroxybutanoate. One exemplary configuration includes, for example, disc stack centrifugation, ultrafiltration, evaporative crystallization, ion exchange, and distillation. Thus, in some embodiments, the present disclosure provides a process of isolating (R)-3-hydroxybutyl (R)-3-hydroxybutanoate from a fermentation broth that includes removing a portion of solids by disc stack centrifugation to provide a liquid fraction, removing a further portion of solids from the liquid fraction by ultrafiltration, removing a portion of salts from the liquid fraction by evaporative crystallization, removing a further portion of salts from the liquid fraction by ion exchange, and distilling (R)-3-hydroxybutyl (R)-3-hydroxybutanoate.

Cells and solids are first removed by disc stack centrifugation. The cells can be optionally recycled back into fermentation. Ultrafiltration removes cell debris, DNA, and precipitated proteins. Evaporative crystallization removes a portion of the media salts and water, either of which can be optionally recycled back into fermentation. Following evaporative crystallization, the remaining liquid phase is passed through an ion exchange column to remove further salts. After ion exchange, a portion of the water can be evaporated in an evaporator system, as described above. Distillation of the light fraction, is followed by distillation of (R)-3-hydroxybutyl (R)-3-hydroxybutanoate to provide substantially pure (R)-3-hydroxybutyl (R)-3-hydroxybutanoate.

Another exemplary configuration includes disc stack centrifugation, ultrafiltration, nanofiltration, ion exchange, evaporation, and distillation. Thus, in some embodiments, the present disclosure provides a process of isolating (R)-3-hydroxybutyl (R)-3-hydroxybutanoate from a fermentation broth that includes removing a portion of solids by disc stack centrifugation to provide a liquid fraction, removing a further portion of solids from the liquid fraction by ultrafiltration, removing a portion of salts from the liquid fraction by nanofiltration, removing a further portion of salts from the liquid fraction by ion exchange, evaporating a portion of water, and distilling (R)-3-hydroxybutyl (R)-3-hydroxybutanoate.

Cells and solids are first removed by disc stack centrifugation. The cells can be optionally recycled back into fermentation. Ultrafiltration removes cell debris, DNA, and precipitated proteins. Nanofiltration removes a portion of the media salts, which can be optionally recycled back into fermentation. Following nanofiltration, the permeate is passed through an ion exchange column to remove further salts. After ion exchange, a portion of the water can be evaporated in an evaporator system, as described above. Distillation of the light fraction, is followed by distillation of (R)-3-hydroxybutyl (R)-3-hydroxybutanoate to provide substantially pure (R)-3-hydroxybutyl (R)-3-hydroxybutanoate.

The compound of interest can be any compound for which the product can be engineered for biosynthesis in a microorganism. The processes disclosed herein are applicable to compounds of interest that have boiling points higher than water. Specifically, compounds of interest can have a boiling point between about 120° C. and 400° C. Other properties include high solubility or miscibility in water and the inability to appreciably solubilize salts (when employing evaporative crystallization), and neutral compounds with molecular weights below about 100-150 Daltons (for suitability with nanofiltration).

The processes and principles described herein can be applied to isolate a compound of interest from a fermentation broth, where the compound of interest has the general properties described above. Such a process includes separating a liquid fraction enriched in the compound of interest from a solid fraction that includes the cell mass, followed by water and salt removal, followed by purification.

In some embodiments disclosed herein is a process for recycling components of a fermentation broth. The fermentation broth can include (R)-3-hydroxybutyl (R)-3-hydroxybutanoate or any compound of interest having a boiling point higher than water, cells capable of producing (R)-3-hydroxybutyl (R)-3-hydroxybutanoate or the compound of interest, media salts, and water. The process includes separating a liquid fraction enriched in (R)-3-hydroxybutyl (R)-3-hydroxybutanoate or the compound of interest from a solid fraction that includes the cells. The cells are then recycled into the fermentation broth. Water can be removed before or after separation of salts from the liquid fraction. Evaporated water from the liquid fraction is recycled into the fermentation broth. Salts from the liquid fraction can be removed and recycled into the fermentation broth either by removal of water from the liquid fraction, causing the salts to crystallize, or by nanofiltration and/or ion exchange. The separated salts from nanofiltration are then recycled into the fermentation broth. The process provides (R)-3-hydroxybutyl (R)-3-hydroxybutanoate or other compounds of interest which can be further purified by, for example, by distillation.

In some embodiments, a process for producing a compound of interest, such as (R)-3-hydroxybutyl (R)-3-hydroxybutanoate, includes culturing a compound-producing microorganism in a fermentor for a sufficient period of time to produce the compound of interest. The organism includes a microorganism having a compound pathway including one or more exogenous genes encoding a compound pathway enzyme and/or one or more gene disruptions. The process for producing the compound also includes isolating the compound by a process that includes separating a liquid fraction enriched in compound of interest from a solid fraction including cells, removing water from the liquid fraction, removing salts from the liquid fraction, and purifying the compound of interest. The compound of interest has a boiling point higher than water.

In a specific embodiment, a process for producing (R)-3-hydroxybutyl (R)-3-hydroxybutanoate includes culturing a (R)-3-hydroxybutyl (R)-3-hydroxybutanoate-producing microorganism in a fermentor for a sufficient period of time to produce (R)-3-hydroxybutyl (R)-3-hydroxybutanoate. The organism includes a microorganism having a (R)-3-hydroxybutyl (R)-3-hydroxybutanoate pathway including one or more exogenous genes encoding a compound pathway enzyme and/or one or more gene disruptions. The process for producing (R)-3-hydroxybutyl (R)-3-hydroxybutanoate also includes isolating the compound by a process that includes separating a liquid fraction enriched in compound of interest from a solid fraction including cells, removing water from the liquid fraction, removing salts from the liquid fraction, and purifying the compound of interest.

In particular embodiments where the product of interest is (R)-3-hydroxybutyl (R)-3-hydroxybutanoate, production begins with the culturing of a microbial organism capable of producing (R)-3-hydroxybutyl (R)-3-hydroxybutanoate via a set of (R)-3-hydroxybutyl (R)-3-hydroxybutanoate pathway enzymes. Exemplary microbial organisms include, without limitation, those described in U.S. 2009/0075351 and U.S. 2009/0047719, both of which are incorporated herein by reference in their entirety.

Organisms can be provided that incorporate one or more exogenous nucleic acids that encode enzymes in a (R)-3-hydroxybutyl (R)-3-hydroxybutanoate pathway. Such organisms include, for example, non-naturally occurring microbial organisms engineered to have a complete (R)-3-hydroxybutyl (R)-3-hydroxybutanoate biosynthetic pathway. Such pathways can include enzymes encoded by both endogenous and exogenous nucleic acids. Enzymes not normally present in a microbial host can add in functionality to complete a pathways by including one or more exogenous nucleic acids, for example. One such (R)-3-hydroxybutyl (R)-3-hydroxybutanoate pathway includes enyzmes encoding a 4-hydroxybutanoate dehydrogenase, a succinyl-CoA synthetase, a CoA-dependent succinic semialdehyde dehydrogenase, a 4-hydroxybutyrate:CoA transferase, a 4-butyrate kinase, a phosphotransbutyrylase, an α-ketoglutarate decarboxylase, an aldehyde dehydrogenase, an alcohol dehydrogenase or an aldehyde/alcohol dehydrogenase.

Prior to culturing the compound-producing or (R)-3-hydroxybutyl (R)-3-hydroxybutanoate-producing organisms, the raw materials feedstock such as sucrose syrup and media components can be treated, for example, by heat sterilization prior to addition to the production bioreactor to eliminate any biological contaminants. In accordance with some embodiments, the feedstock can include, for example, sucrose or glucose for the fermentation of (R)-3-hydroxybutyl (R)-3-hydroxybutanoate. In some embodiments, the feedstock can include syngas. Additional media components used to support growth of the microorganisms include, for example, salts, nitrogen sources, buffers, trace metals, and a base for pH control. The major components of an exemplary media package, expressed in g/L of fermentation broth, are shown below in Table 1.

TABLE 1

| Category | Concentration (g/L) |
|---|---|
| N-Source | 3 |
| Buffer | 5 |
| Salts | 0.65 |
| Base | 1.4 |
| Carbon Source | 10.1 |

The type of carbon source can vary considerably and can include glucose, fructose, lactose, sucrose, maltodextrins, starch, inulin, glycerol, vegetable oils such as soybean oil, hydrocarbons, alcohols such as methanol and ethanol, organic acids such as acetate, syngas, and similar combinations of CO, CO₂, and H₂. The term “glucose” includes glucose syrups, i.e. glucose compositions including glucose oligomers. Plant and plant-derived biomass material can be a source of low cost feedstock. Such feedstock can include, for example, corn, soybeans, cotton, flaxseed, rapeseed, sugar cane and palm oil. Biomass can undergo enzyme or chemical mediated hydrolysis to liberate substrates which can be further processed via biocatalysis to produce chemical products of interest. These substrates include mixtures of carbohydrates, as well as aromatic compounds and other products that are collectively derived from the cellulosic, hemicellulosic, and lignin portions of the biomass. The carbohydrates generated from the biomass are a rich mixture of 5 and 6 carbon sugars that include, for example, sucrose, glucose, xylose, arabinose, galactose, mannose, and fructose.

The carbon source can be added to the culture as a solid, liquid, or gas. The carbon source can be added in a controlled manner to avoid stress on the cells due to overfeeding. In this respect, fed-batch and continuous culturing are useful culturing modes as further discussed below.

The type of nitrogen source can vary considerably and can include urea, ammonium hydroxide, ammonium salts, such as ammonium sulphate, ammonium phosphate, ammonium chloride and ammonium nitrate, other nitrates, amino acids such as glutamate and lysine, yeast extract, yeast autolysates, yeast nitrogen base, protein hydrolysates (including, but not limited to, peptones, casein hydrolysates such as tryptone and casamino acids), soybean meal, Hy-Soy, tryptic soy broth, cotton seed meal, malt extract, corn steep liquor and molasses.

The pH of the culture can be controlled by the addition of acid or alkali. Because pH can drop during culture, alkali can be added as necessary. Examples of suitable alkalis include NaOH and NH₄OH.

Exemplary cell growth procedures used in the production of a compound of interest, such as (R)-3-hydroxybutyl (R)-3-hydroxybutanoate, include, batch fermentation, fed-batch fermentation with batch separation; fed-batch fermentation with continuous separation, and continuous fermentation with continuous separation. All of these processes are well known in the art. Depending on the organism design, the fermentations can be carried out under aerobic or anaerobic conditions. In some embodiments, the temperature of the cultures kept between about 30 and about 45° C., including 31, 32, 33, 34, 35, 36, 37, 38, 39, 40, 41, 42, 43, and 44° C.

In batch fermentation, a tank fermenter (or bioreactor) is filled with the prepared media to support growth. The temperature and pH for microbial fermentation is properly adjusted, and any additional supplements are added. An inoculum of a (R)-3-hydroxybutyl (R)-3-hydroxybutanoate-producing organism is added to the fermenter. In batch fermentation the fermentation will generally run for a fixed period and then the products from the fermentation are isolated. The process can be repeated in batch runs.

In fed-batch fermentation fresh media is continuously or periodically added to the fermentation bioreactor. Fixed-volume fed-batch fermentation is a type of fed-batch fermentation in which a carbon source is fed without diluting the culture. The culture volume can also be maintained nearly constant by feeding the growth carbon source as a concentrated liquid or gas. In another type of fixed-volume fed-batch culture, sometimes called a cyclic fed-batch culture, a portion of the culture is periodically withdrawn and used as the starting point for a further fed-batch process. Once the fermentation reaches a certain stage, the culture is removed and the biomass is diluted to the original volume with sterile water or medium containing the carbon feed substrate. The dilution decreases the biomass concentration and results in an increase in the specific growth rate. Subsequently, as feeding continues, the growth rate will decline gradually as biomass increases and approaches the maximum sustainable in the vessel once more, at which point the culture can be diluted again. Alternatively, a fed-batch fermentation can be variable volume. In variable-volume mode the volume of the fermentation broth changes with the fermentation time as nutrient and media are continually added to the culture without removal of a portion of the fermentation broth.

In a continuous fermentation, fresh media is generally continually added with continuous separation of spent medium, which can include the product of interest, such as (R)-3-hydroxybutyl (R)-3-hydroxybutanoate, when the product is secreted. One feature of the continuous culture is that a time-independent steady-state can be obtained which enables one to determine the relations between microbial behavior and the environmental conditions. Achieving this steady-state is accomplished by means of a chemostat, or similar bioreactor. A chemostat allows for the continual addition of fresh medium while culture liquid is continuously removed to keep the culture volume constant. By altering the rate at which medium is added to the chemostat, the growth rate of the microorganism can be controlled.

The continuous and/or near-continuous production of a compound of interest, such as (R)-3-hydroxybutyl (R)-3-hydroxybutanoate can include culturing a compound-producing organism in sufficient nutrients and medium to sustain and/or nearly sustain growth in an exponential phase. Continuous culture under such conditions can include, for example, 1 day, 2, 3, 4, 5, 6 or 7 days or more. Additionally, continuous culture can include 1 week, 2, 3, 4 or 5 or more weeks and up to several months. Alternatively, organisms that produce a compound of interest can be cultured for hours, if suitable for a particular application. It is to be understood that the continuous and/or near-continuous culture conditions also can include all time intervals in between these exemplary periods. It is further understood that the time of culturing the compound-producing microbial organism is for a sufficient period of time to produce a sufficient amount of product for a desired purpose.

In some embodiments, the culture can be conducted under aerobic conditions. An oxygen feed to the culture can be controlled. Oxygen can be supplied as air, enriched oxygen, pure oxygen or any combination thereof. Methods of monitoring oxygen concentration are known in the art. Oxygen can be delivered at a certain feed rate or can be delivered on demand by measuring the dissolved oxygen content of the culture and feeding accordingly with the intention of maintaining a constant dissolved oxygen content. In other embodiments, the culture can be conducted under substantially anaerobic conditions. Substantially anaerobic means that the amount of oxygen is less than about 10% of saturation for dissolved oxygen in liquid media. Anaerobic conditions include sealed chambers of liquid or solid medium maintained with an atmosphere of less than about 1% oxygen.

Fermentations can be performed under anaerobic conditions. For example, the culture can be rendered substantially free of oxygen by first sparging the medium with nitrogen and then sealing culture vessel (e.g., flasks can be sealed with a septum and crimp-cap). Microaerobic conditions also can be utilized by providing a small hole for limited aeration. On a commercial scale, microaerobic conditions are achieved by sparging a fermentor with air or oxygen as in the aerobic case, but at a much lower rate and with tightly controlled agitation.

In some embodiments, the compound of interest, including (R)-3-hydroxybutyl (R)-3-hydroxybutanoate, can be produced in an anaerobic batch fermentation using genetically modified E. Coli. In fermentation, a portion of the feedstock substrate is used for cell growth and additional substrate is converted to other fermentation byproducts. Media components such as salts, buffer, nitrogen, etc can be added in excess to the fermentation to support cell growth. The fermentation broth is thus a complex mixture of water, the compound of interest, byproducts, residual media, residual substrate, and feedstock/media impurities. It is from this fermentation broth that the compound of interest is isolated and purified. An exemplary fermentation broth composition is shown below in Table 2.

TABLE 2

| | |
|---|---|
| Quantity | Component |
| ~100 g/L | (R)-3-hydroxybutyl (R)-3-hydroxybutanoate |
| ~5 g/L | cell mass |
| ~10 g/L | byproducts (ethanol, acetic acid, 4-hydroxybutyric acid, GBL, proteins) |
| <10 g/L | residual media/salts |
| <1 g/L | residual sucrose/glucose |
| <2 g/L | “unfermentables” (feedstock/impurities) |

*Balance water

A product concentration of about 5-15% by weight of (R)-3-hydroxybutyl (R)-3-hydroxybutanoate can be achieved through fermentation based biosynthetic production processes.

It is understood that modifications which do not substantially affect the activity of the various embodiments of this disclosure are also included within the definition of the disclosure provided herein. Accordingly, the following examples are intended to illustrate but not limit the present disclosure.

### Example 1

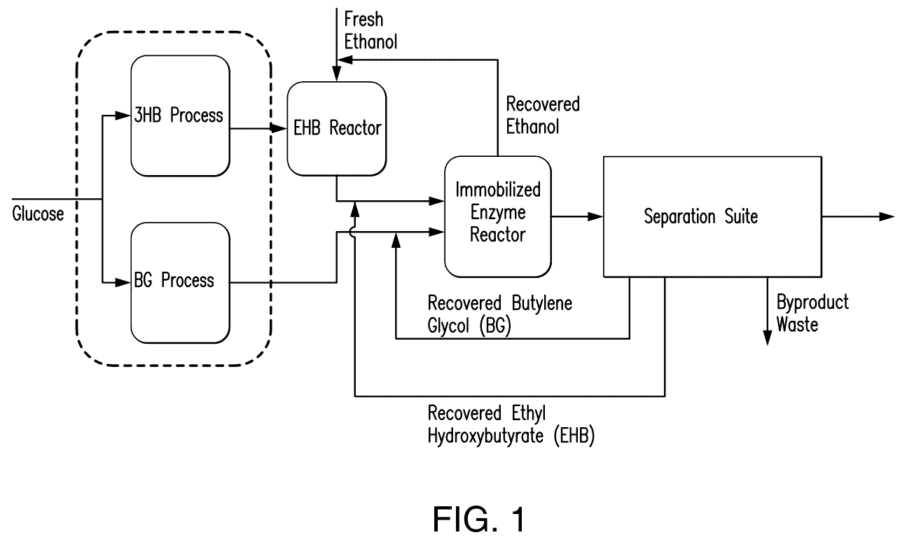

This examples shows a process for the production and purification of (3R)-hydroxybutyl (3R)-hydroxybutyrate (Ketone Ester) from sugar. In this process sugar and makeup ethanol are provided as the feedstock to the unit and Ketone Ester is produced as the product. The overall process consists of five major steps shown in FIG. 1:
1. Fermentation of glucose to (R)-1,3-butanediol (BG);
2. Fermentation of glucose to (R)-3-hydroxybutyric acid (3HB);
3. Fischer Esterification 3HB and ethanol to (R)-ethyl-3-hydroxybutyrate (E3HB) in the presence of a strong acid;
4. Enzymatic transesterification of E3HB and BG to Ketone Ester in presence of an immobilized enzyme (i.e. Lipase or esterase); and
5. Separation and purification of the Ketone Ester product.

The two fermentation processes (steps 1 and 2) can be designed and operated to utilize the same fermentation and ancillary equipment when possible to maximize capital efficiency. Esterification of (R)-3-hydroxybutanoic acid (3HB) to ethyl (R)-3-hydroxybutanoate (E3HB) takes place according to the following reaction:

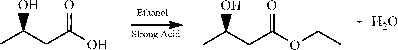

To help drive this reaction toward completion, excess reactant 3HB is introduced to the reactor. Excess 3HB is recovered and recycled to the reactor in order to minimize the loss of 3HB. The required ethanol for this reaction is supplied by recycling ethanol that is generated from transesterification of 3HB with BG to E3HB:

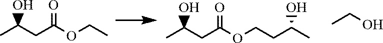

The stoichiometry of the reactions suggests that produced ethanol in this step is sufficient to complete the Fischer Esterification reaction. Makeup ethanol, however, may be required to compensate for the ethanol losses during the separation processes. In order to improve the conversion, transesterification reaction is also carried out with excess amount of the reactant, E3HB. Excess E3HB and unreacted (R)-butane-1,3-diol (BG) are recovered in downstream separation units and recycled to the reactor to improve the conversion.

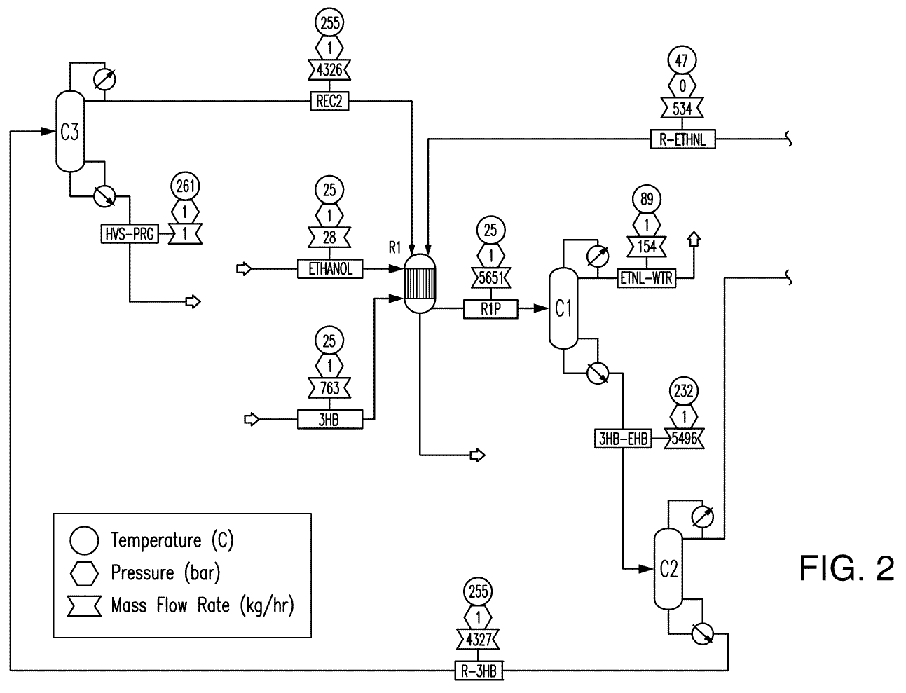

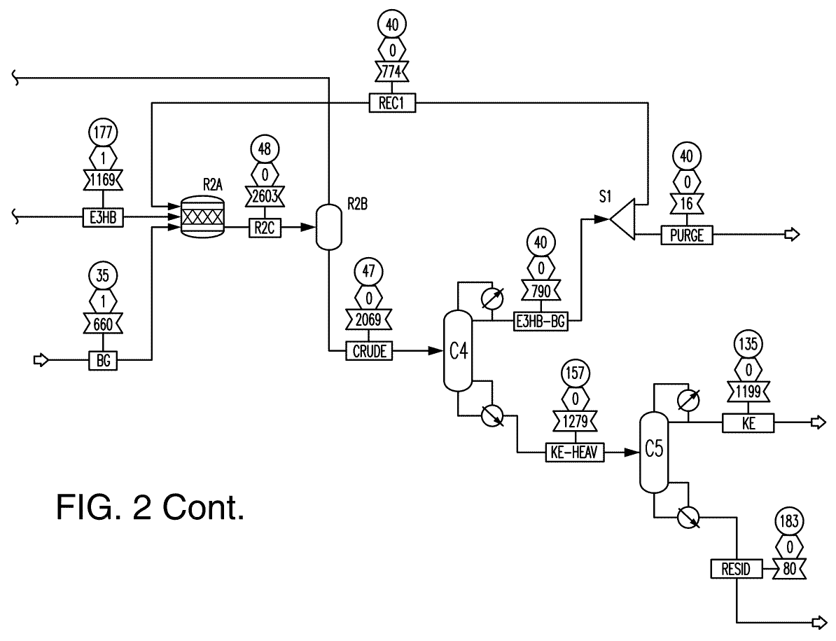

FIG. 2 shows the schematic of an integrated process of the Fischer Esterification reaction, Enzymatic Transesterification reaction, and downstream separation units. Through this integrated system, 3HB, BG, and makeup ethanol are provided as feedstock to the unit and Ketone Ester is produced and purified as the final product. Conversion of excess 3HB and ethanol to E3HB takes place in R1. Column C1 is utilized to separate and remove the produce water out of the system. Water removal involves in some loss of ethanol which is compensated by the makeup steam. E3HB product is recovered through the distillate stream of column C2 and is sent to R2A for Ketone Ester production. Unreacted 3HB is recovered through the bottom stream of column C2 and recycled to R1 reactor. Column C3 provides an opportunity to purge the heavy boilers out of the 3HB recycle loop. This column can be replaced with a regular purge stream in the cost of losing some 3HB along with the heavy boilers.

Transesterification of E3HB to Ketone Ester takes place in R2A. The ethanol produced by this reaction is recovered by a regular flash separator and recycled to the Fischer Esterification reactor, R1. 0.93 moles of Ketone Ester are produced per 1 mole of BG and 1 mole of 3HB fed to the system. Assuming 80% yield for fermentation and purification of BG and 3HB from Glucose, the overall yield of Ketone Ester production from glucose can be estimated as 0.36 kg Ketone Ester per 1 kg of glucose.

Conceptually steps 3 and 4 can be considered to be combined in one reactor, where 3HB reacts with BG to produce KE and water. The reaction may need ethanol present in the beginning to initiate the reaction. And it may need acid and other catalytic components to accelerate the production of KE. If it is feasible, this option will reduce two reactions to one reaction, remove the need of ethanol recycling and all the capital equipment associated with separation and recycling.

3HB and BG can be converted to Ketone Ester by the following chemical reaction in presence of an acid catalyst.

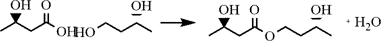

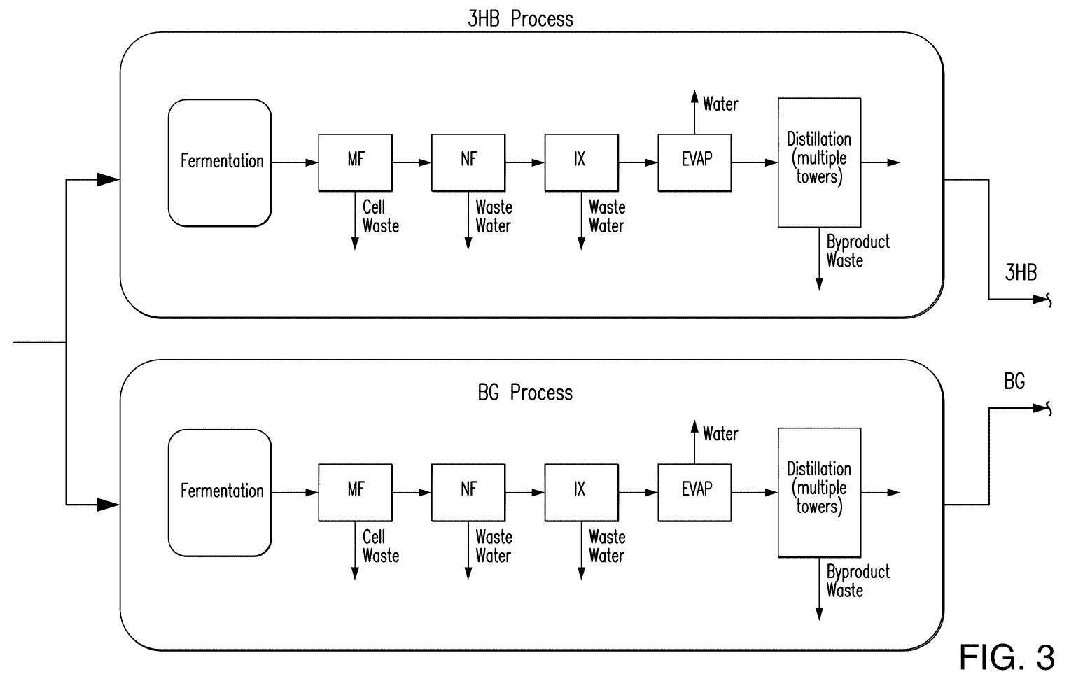

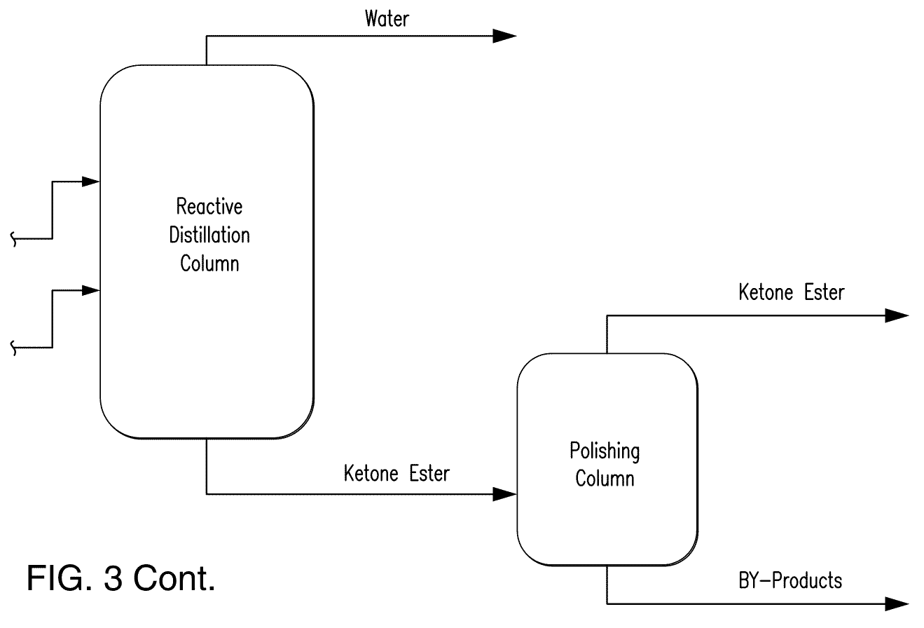

The equilibrium constant of this reaction is about 0.5 at 25° C. That would lead to only 33% conversion of 3HB to KE at 25° C. Conversion of this reaction, however, can be significantly improved by continuous withdrawal of water from the product mixture as it forms. The chemical reaction and separation and removal of water can be combined in a reactive distillation column as shown in FIG. 3. Aspen plus simulation models shows that 99% conversion of 3HB to KE can be achieved by equimolar feeding of 3HB and BG and proper design and operation of the column.

In this process reaction takes places on the stage of the column in liquid phase. Produced water is vaporized as soon as it forms and leaves the liquid phase which is the reaction phase. Also, liquid phase on each stage of the column acts as one reactor. The combined effects of continuous water removal and multiple stages of reaction lead to the significantly higher yield and rate of the reaction. As a result, required residence time for the reaction significantly decreases. It would also eliminate the requirements of separating and recycling the unreacted reactants from the product.

As shown in FIG. 3, water is removed from the top of the column and pure Ketone Ester is recovered from the bottom of the column. With proper design, operation, and control of the column no unreacted BG and 3HB will be produced. However, a polishing column might be needed to remove unwanted by products, other impurities, or dissolved acid if liquid acid has been used. Recovered acid in polishing column can be recycled to the column for re-use as the acid catalyst. Alternatively, reactive distillation column can be packed with solid acid catalysts in order to avoid using liquid acid and consequent recovery in polishing column.

### Example 2

This examples shows a process for production and purification of (3R)-hydroxybutyl (3R)-hydroxybutyrate (Ketone Ester) with high quality and at a high yield.

Ketone Ester is produced by enzymatic transesterification of (R)-Ethy-3Hydroxybutyrate (E3HB) and (R)-1,3-butanediol (BG) in presence of immobilized enzyme (i.e. Lipase or esterase) as the catalyst.

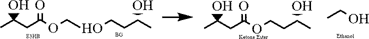

Purification of the product is achieved through series of distillation columns and wiped film evaporators.

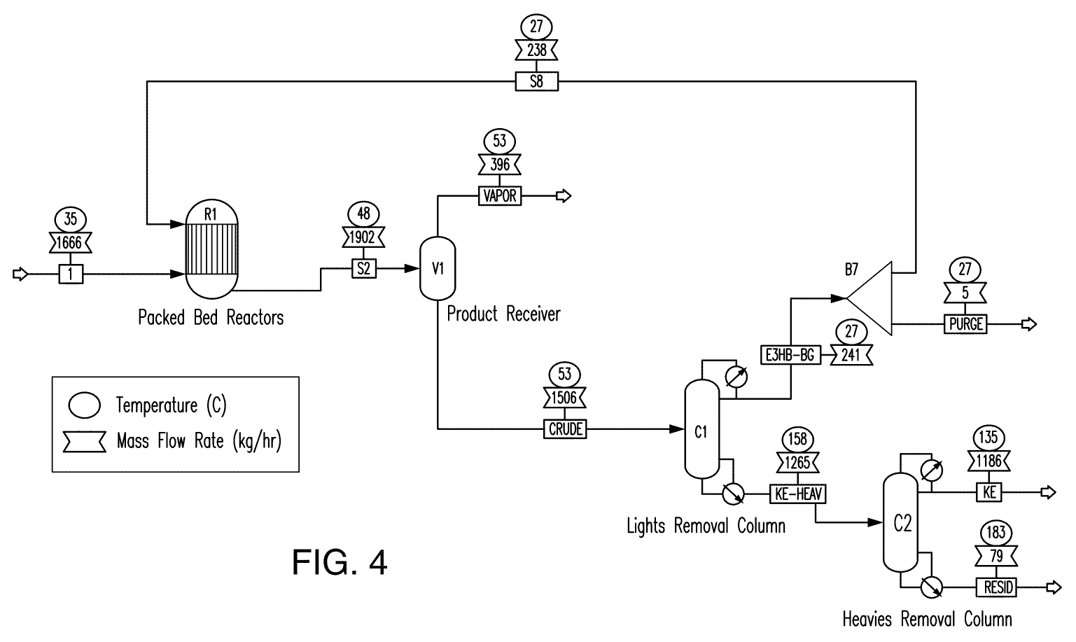

FIG. 4 shows the overall process for production and purification of Ketone Ester. In this process BG and E3HB are provided as feed to the reactor while produced ethanol is purged through the gas phase. The bottom product of the reactor which is a mixture of Ketone Ester and unreacted BG and E3HB is sent to the separation units for Ketone Ester recovery.

The reactor can be designed and optimized to maximize performance and reduce cost. For example a CSTR type of reactor can be designed where ethanol is purged from the top of the reactor and liquid product is recovered from the bottom. Alternatively, the reaction system can be designed as two or more parallel packed bed reactors and a receiving tank to collect the reaction product and separate the ethanol from the liquid mixture.

The reaction operation condition can be optimized as well. Operating under vacuum condition (i.e. 10 to 20 Torr), for example can help increasing the efficiency of ethanol removal and therefore prevent the reverse reaction to take place. Also, running the reactor under the vacuum condition can help to the lower operating temperature of the reactor to decrease the production of impurities under high temperature. On the other hand running the reactor at lower temperature can adversely impact the reaction rate, and so increase the reactor volume and corresponding capital equipment cost.

Column C1 is utilized to recover the unreacted BG and E3HB from the reaction product and recirculate them back to the reactor. With proper design of the distillation column (i.e. temperature, pressure, number of stages, reflux, reboiler, etc) 97% or above of BG and E3HB in reaction product can be recovered. Since some heavy boilers are carried over with the distillate product of the column C1 a small purge stream is required to purge these heavy boilers. This purge stream can be minimized at as low as 0.3% of the reaction product.

Column C2 is utilized to separate Ketone Ester from heavy boilers. Polished Ketone Ester product is recovered through the distillate stream and components with higher boiling points are separated through the bottom product. Vacuum condition may be desired to prevent discoloration and change in quality of Ketone Ester product.

With proper design of the distillation section 99% of Ketone Ester in reaction product can be recovered. When combined with the yield from the reaction section, 91.3% of the theoretical yield (kg of Ketone Ester per kg of BG) can be achieved. In this process the purity of final Ketone Ester product is as high as 99.4%.

### Example 3

This examples shows the production of KE from E3HB and BG. Produced E3HB then can be further processed and purified through downstream separation units

Similarly, BG can be produced through the metabolic pathway inside the cells and purified through downstream separation units.

Ketone Ester is then produced by enzymatic transesterification of (R)-Ethyl-3-hydroxybutyrate (E3HB) and (R)-1,3-butanediol (BG) in the presence of immobilized enzyme (i.e. Lipase or esterase) as the catalyst.

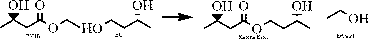

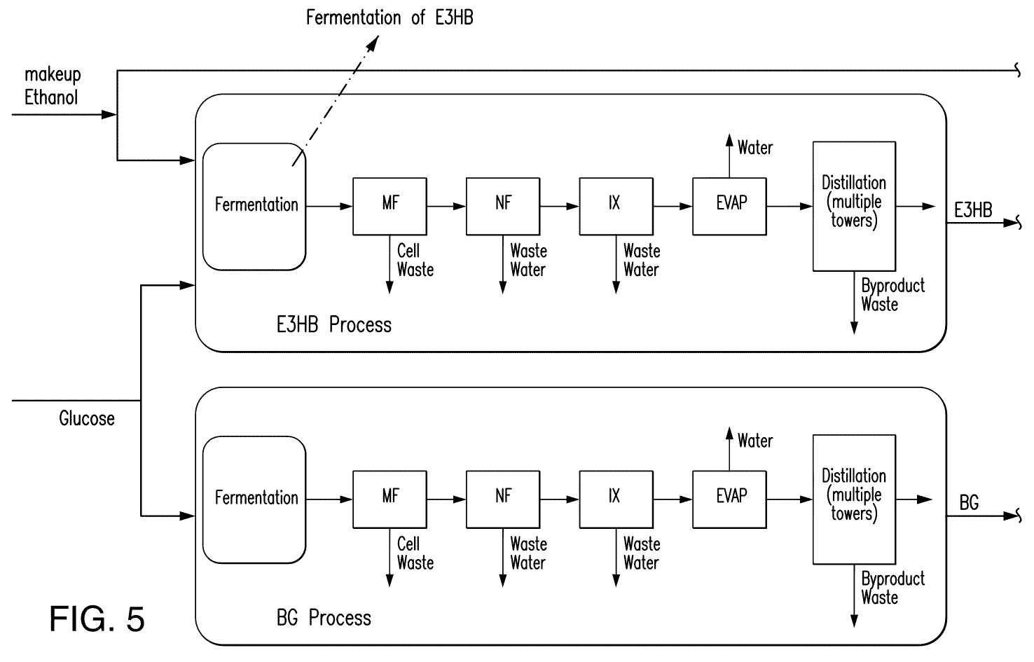

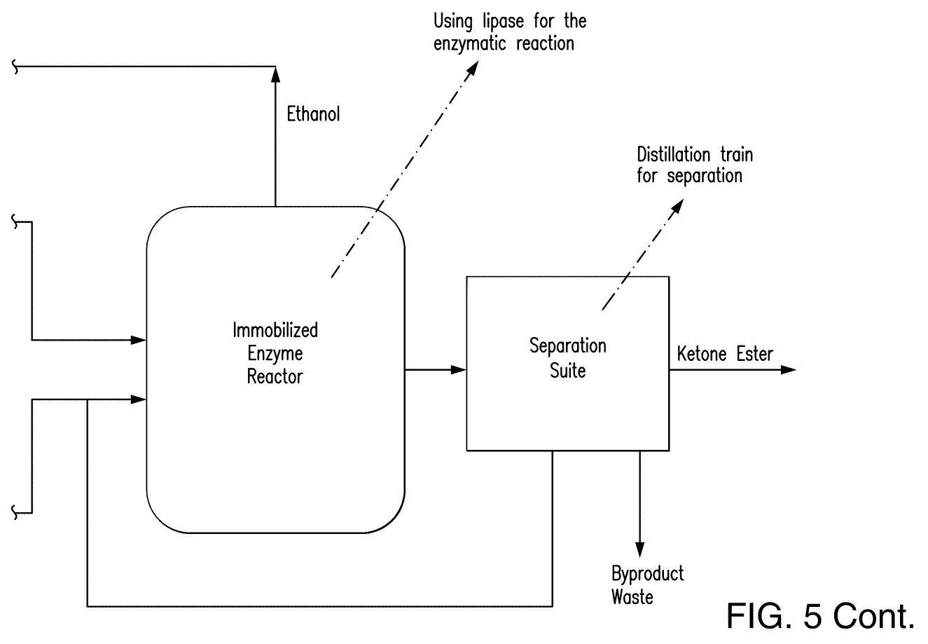

FIG. 5 shows the overall process for production and purification of Ketone Ester through this process. In this process glucose and ethanol are introduced as feed to the fermenter where selected organism converts glucose and ethanol to E3HB and transport out of the cells. Fermentation broth would be a mixture of water, E3HB, unfermented sugar(s) and ethanol, cells, nutrient, organic acids, as well as macromolecules, and other by products. Fermentation broth which contains certain concentration of E3HB, and water is sent to downstream separation units for product purification and recovery. Downstream separation (DSP) units for E3HB would be similar to the DSP units of direct KE fermentation. BG is also produced and purified.

Purified E3HB and BG are converted to Ketone Ester by enzymatic transesterification reaction in presence of immobilized enzyme (i.e. Lipase or esterase) as the catalyst. As shown in FIG. 5, recovered ethanol in this step can be recycled to the fermenter for E3HB production. Make-up ethanol is required to compensate for the ethanol losses though the process.

Alternatively, chemical conversion of E3HB and BG to Ketone Ester in a presence of a proper catalyst can be carried out in a reactive distillation column:

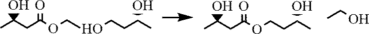

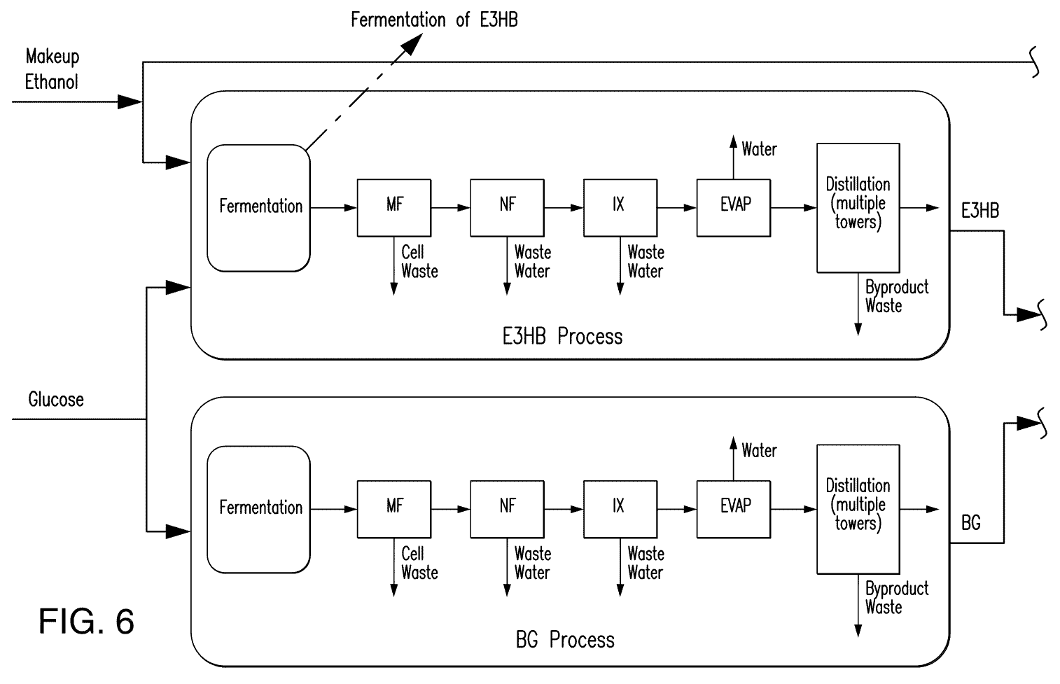

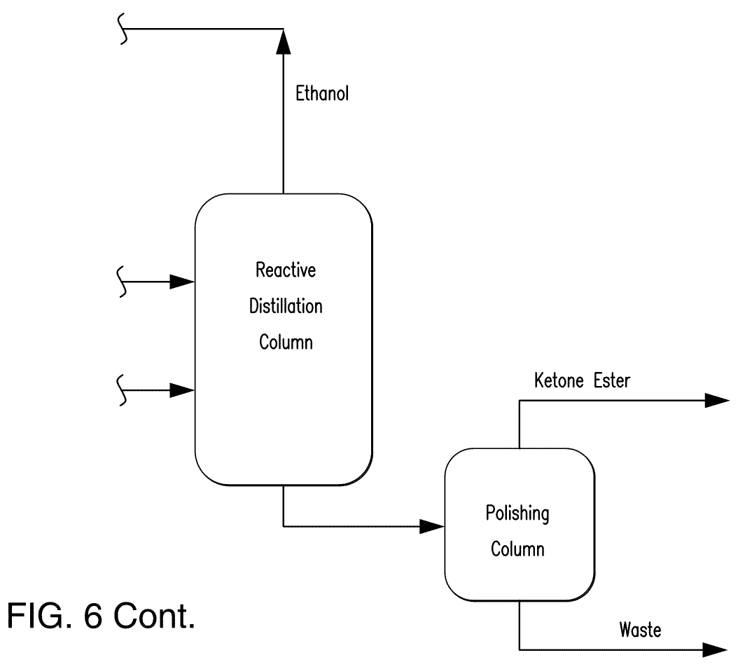

The low equilibrium constant of this reaction leads to a lower conversion and therefore, a bigger reactor along with separation and recycle of unreacted reactant is needed. Conversion of this reaction, however, can be significantly improved by continuous withdrawal of ethanol from the product mixture as it forms. The chemical reaction and separation and removal of ethanol can be combined in a reactive distillation column as shown in FIG. 6.

In this the process reaction takes places on the stage of the column in liquid phase. The produced ethanol is vaporized as soon as it forms and leaves the liquid phase which is the reaction phase. Also, the liquid phase on each stage of the column acts as one reactor. The combined effect of continuous ethanol removal and multiple stages of reaction leads to the significantly higher yield and rate of the reaction. As a result, required residence time for the reaction significantly decreases. It would also eliminate the requirements of separating and recycling the unreacted reactants from the product.

As shown in FIG. 6, ethanol is removed from the top of the column and pure Ketone Ester is recovered from the bottom of the column. With proper design, operation, and control of the column no unreacted BG and E3HB will be produced. However, a polishing column might be needed to remove unwanted by products, other impurities.

### Example 4

This example shows a process for the production and purification of (3R)-hydroxybutyl (3R)-hydroxybutyrate (Ketone Ester, KE) from sugar fermentation.

(3R)-hydroxybutyl (3R)-hydroxybutyrate (Ketone Ester) can be produced through the metabolic pathway inside the cells with consumption of glucose and (R)-1,3-butanediol (BG). In this pathway, glucose will be converted to (R)-ethy-3-hydroxybutyrateCoA plus an esterase inside the cells. Then, cells take up the BG and combined that with (R)-Ethy-3Hydroxybutyrate (E3HB) to produce the Ketone Ester.

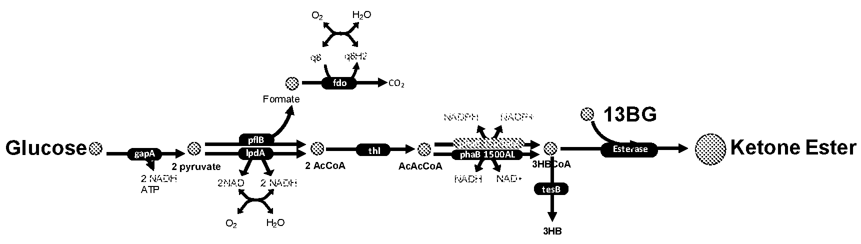

The produced Ketone Ester then can be further processed and purified through downstream separation units.

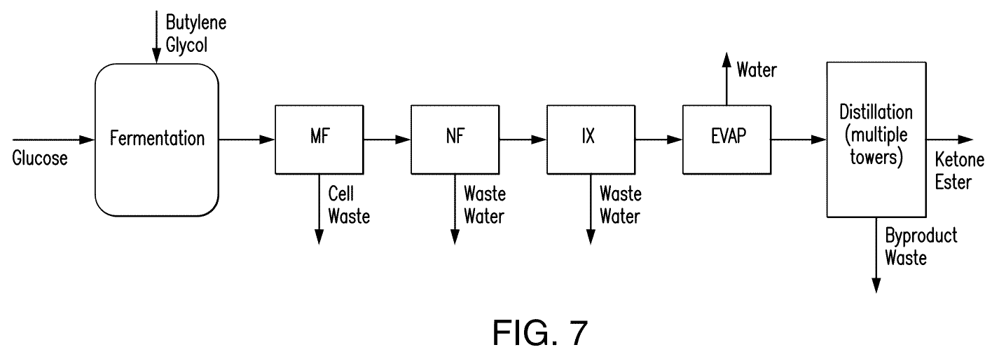

FIG. 7 shows the overall process for production and purification of Ketone Ester through direct fermentation. In this process glucose and BG are introduced as feed to the fermenter where a selected organism converts glucose and BG to Ketone Ester. The fermentation broth would be a mixture of water, ketone ester, unfermented sugar(s) and BG, cells, nutrient, organic acids, as well as macromolecules, and other by products.

Fermentation broth which contains certain concentrations of KE (ie. 5 to 10 wt%) and water (i.e. 85-90 wt%) is sent to downstream separation units for product purification and recovery.

In the first step, Micro and Nano filtrations (MF and NF), or alternatively, Ultra and Nano filtrations, are utilized to remove the cells and macromolecules. In the second step, cell free product is processed through the ion exchange units to remove the ionic species (i.e. Ca⁺, Mg²⁺, PO₄³⁻, SO₄²⁻, Fe²⁺ and trace metals). Next, a film evaporator is utilized to evaporate the water and decrease the water content of the solution from about 85% to ~15% wt. Steam generated in this unit can be used elsewhere. At this point, the solution is sent through several distillation columns to remove the remaining water, lighter components and heavier components.

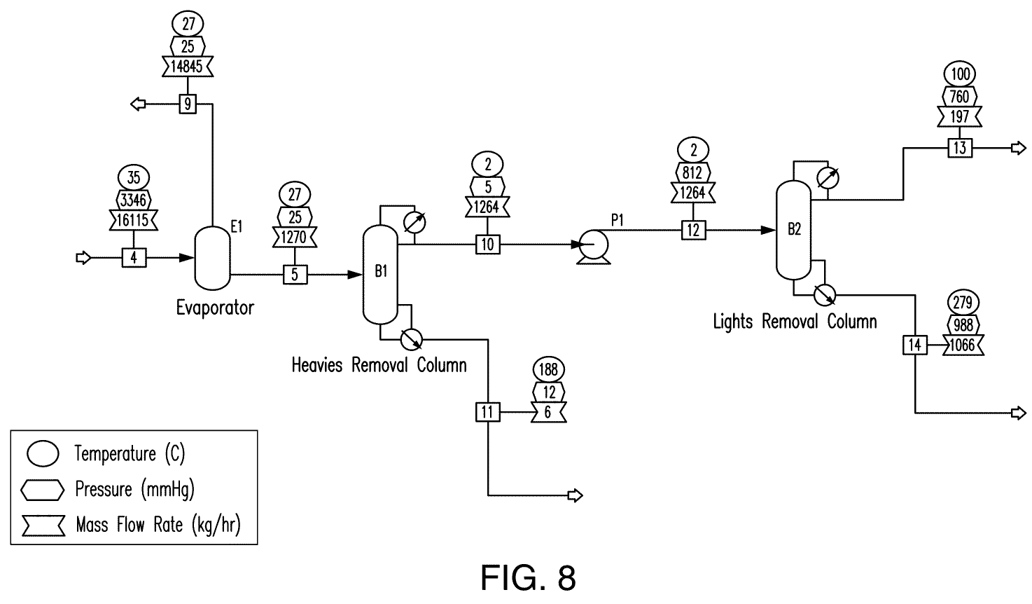

FIG. 8 shows a schematic of the distillation units. Column C1 is utilized to separate Ketone Ester from the heavy boilers. The Ketone Ester product and some lighter materials are recovered through the distillate stream while components with higher boiling points are separated through the bottom product. Column C2 is utilized to separate Ketone Ester from lighter components. The polished Ketone Ester product is recovered through the bottom stream and components with lower boiling points are separated through the distillate product. Distillation columns can be operated under vacuum condition to prevent discoloration and change in quality of Ketone Ester product.

With proper design of the downstream separation units 98.0% or higher percentage of the Ketone Ester in fermentation broth can be recovered. The theoretical yield of the fermentation is 1 mole Ketone Ester per 1 mole of glucose and 1 mole of BG (BG can be produced from fermentation of sugar at a theoretical yield of 1 mole BG per 1 mole of glucose). Therefore, theoretical yield of fermentation can be presented as 1 mole Ketone Ester per 2 moles of glucose.

### Example 5

This example shows a process for purification and separation of (3R)-hydroxybutyl (3R)-hydroxybutyrate (Ketone Ester) from fermentation broth by utilization of a liquid-liquid extraction technique.

(3R)-hydroxybutyl (3R)-hydroxybutyrate (Ketone Ester) is a synthetic chemical compound that also can be produced by biomass fermentation. However, the recovery of a component from its fermentation broth is a challenge due to the low concentration of the KE and limitation of the product solubility in water and other components. Conventional recovery of a product from fermentation broth involves utilization of multiple filtration, ion exchange, and distillation units, as well as evaporation of large amounts of water. This disclosure provides a process for recovery and purification of Ketone Ester from its fermentation broth by applying liquid-liquid extraction (LLE) technique.

Among the list of commercially available solvents there are few solvents that have the right properties for liquid-liquid extraction of KE from the fermentation broth which is dominated by the water phase. These potential solvents are: 1-butanol, 1-hexanol, and tributyl phosphate (TBP). The boiling points of 1-butanol and 1-hexanol are lower than the boiling point of KE while the boiling point of TBP is higher than the boiling point of KE. Therefore, the design of the downstream separation units is different when applying these two groups of the solvents.

A study to determine the boiling point of (3R)-hydroxybutyl (3R)-hydroxybutyrate was performed. The results are displayed in Table 3.

TABLE 3

| Pressure (mbar) | Measured Boiling Point (°C) | Color Change |
|---|---|---|
| 7 | 155-159 | no color change after 2 h of boiling |
| 15 | 165-169 | no color change after 2 h of boiling |
| 30 | 174-179 | no color change after 2 h of boiling |
| 60 | 186-191 | no color change after 2 h of boiling |
| 80 | 197-204 | no color change after 2 h of boiling |
| 120 | 208-212 | no color change after 1 h of boiling, very slight yellow tint is present after 1 h and 30 min of boiling |
| 240 | 215-219 | no color change after 1 h of boiling, very slight yellow tint is present after 1 h and 30 min of boiling |
| 500 | 224-229 | no color change after 1 h of boiling, very slight yellow tint is present after 1 h and 30 min of boiling |
| 1000 | 274-280 | slight yellow tint is present after 1 hour of boiling |

A study to determine the optimal solvents for the liquid-liquid extraction was performed. The following solvents were tested: N-methyl-2-piperidone, trifluoroethanol, N,N-diethylacetamide, 5-(hydroxymethyl)furfural, dimethylacetamide, DMSO, furfurylalcohol, N-methylacetamide, ethyl lactate, N-methylpyrrolidinone, methanol, tetrahydrofurfurylic alcohol, 2-pyrrolidone, DMF, ethanol/water(90:10) vol, 1,2-propyleneglycol, ethanol/water(80:20) vol, glycerol-1,3-diethylether, ethanol, acetic acid, ethanol/water(70:30) vol, glycerol-1,2-diethylether, N-ethylacetamide, Solketal, dipropyleneglycol, N-ethylformamide, glycerol-1,3-dimethylether, 2-butoxy-1,3-propanediol, tributyl phosphate, benzylalcohol, 3-methyl-1-butanol, glycofurol(n=2), glycerol-1-ethylmonoether, glycerol-2-ethylmonoether, glycerol, glycerol-1,2-dimethylether, glycerol-1-methylmonoether, glycerolcarbonate, 2-propanol, Propionic acid, 1-propanol, ethylene, ethanol/water(60:40) vol, glycerol-2-methylmonoether, 1,3-dioxan-5-ol, PEG200, N-methylformamide, 2-pentanol, 2-butanol, 2-methyl-1-propanol, 2-methyl-2-propanol, 1,3-dioxolane-4-methanol, Caprylicaciddiethanolamide, glycerol-1,3-Dibutylether, THF, PEG600, 1-pentanol, Trimethyleneglycol, 1-butanol, 3-hydroxypropionicacid, ethanol/water(50:50) vol, N,N-diethylolcapramide, 3-methoxy-3-methyl-1-butanol, N-formylmorpholine, 1-hexanol, triethylcitrate, dibutylformamide, formamide, glycerol-1,2-dibutylether, 2-furfuraldehyde, Ethylhexyllactate, ethanol/water(40:60) vol, cyclopentylmethyl ether, 1,4-dioxane, Nopol, 1-heptanol, gamma-valerolactone, methyl5-(dimethylamino)2-methyl-oxopentanoate, alpha-terpisneol, beta-terpineol, acetone, dimethylisosorbide, butanone, 1-octanol, glycerol-1,2,3-trimethylether, geraniol, cyclohexanone, methyl acetate, dimethyl succinate, 2-methyltetrahydrofuran, 1-decanol, 1,3-dioxolane, Dihydromyrcenol, ethanol/water(30:70) vol, cyclademol, menthanol, tributylcitrate, sulfolane, N,N-dimethyloctanamide, Isopropylacetate, dimethyl2-methylglutarate, oleic acid, ethyl acetate, ethanol/water(20:80) vol, methylricinoleate, octadecanol, isobutylacetate, ricinoleicacid, glyceroltriacetate, dimethylglutarate, dimethylphthalate, ethanol/water(10:90) vol, diethylsuccinate, glycerol-1,2,3-triethylether, diethyl ether, water, dimethyladipate, isoamylacetate, N,N-dimethyldecanamide, chloroform, butyl, nitromethane, diethylglutarate, n-propyl acetate, methyl tert-butyl ether, diisobutylsuccinate, benzonitrile, diethylphthalate, diisobutylglutarate, 1,8-cineol, Diethyladipate, 1,4-cineol, Diisobutyladipate, acetonitrile, propylene, decamethylcyclo-pentasiloxane, diisoamylsuccinate, dichloromethane, glycerol-1,2,3-tributylether, menthanylacetate, benzylbenzoate, methylabietate, terpineolacetate, menthylacetate, diisooctylsuccinate, isosorbidedioctanoate, methyllinolenate, nitrobenzene, ethyllinolenate, ethyllinoleate, methyllinoleate, acetyltributylcitrate, 1,2-dichloroethane, Methyloleate, dioctylsuccinate, ethyloleate, dibutylsebacate, geranylacetate, methylstearate, ethylpalmitate, isopropylpalmitate, butylmyristate, methylpalmitate, butyl stearate, butylpalmitate, ethylmyristate, ethyllaurate, methyllaurate, butyllaurate, methylmyristate, fluorobenzene, peanut, benzene, dibutyl, chlorobenzene, isopropyl, bromobenzene, perfluorooctane, toluene, 1-chlorobutane, Iodobenzene, o-xylene, m-xylene, p-xylene, ethylbenzene, carbon, 1,9-decadiene, p-cymene, alpha-pinene, beta-pinene, terpinolene, methylcyclohexane, d-limonene, beta-myrcene, beta-farnesen, carbon, 1-hexadecene, hexane, cyclohexane, nonane, heptane, isododecane, 2,2,4-trimethylpentane, decane, pentane, Octane, hexadecane, endecane, and dodecane.

Of these, 2-butoxy-1,3-propanediol, tributyl phosphate, 1-pentanol, 1-hexanol, and triethylcitrate had the appropriate features of partition coefficient and low solubility in water.

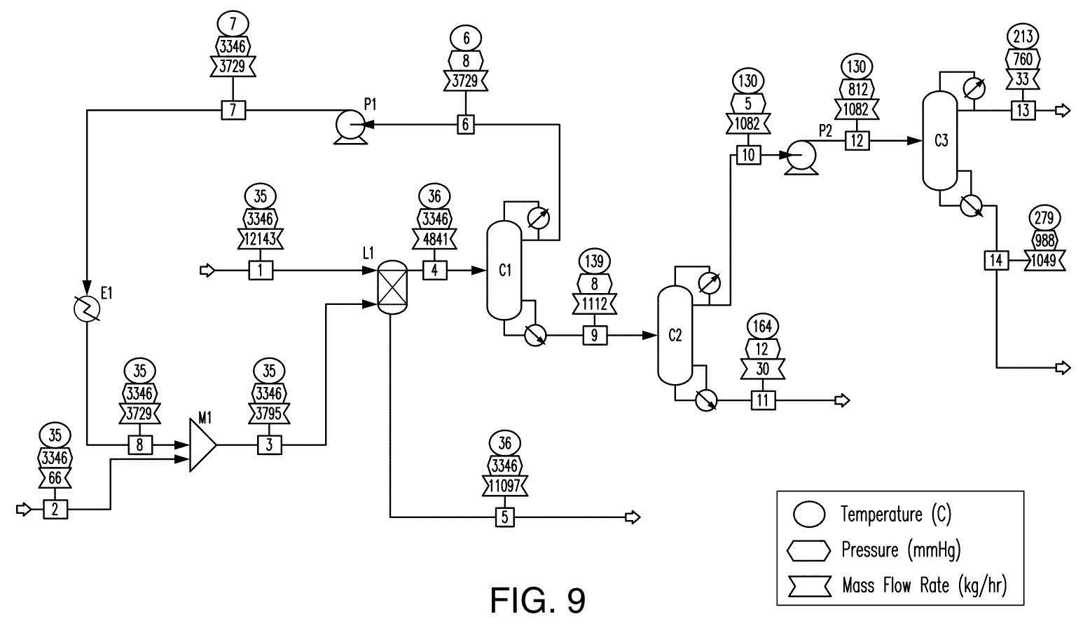

FIG. 9 shows the process for recovery of KE from the fermentation broth when a lower boiling point solvent (i.e. 1-hexanol) is used. In this process cell free fermentation broth (stream 1) is brought in contact with a recirculating solvent through the solvent contact column L1. The solvent contact column can operate under ambient temperature and atmospheric pressure. With proper design of the contact column high purity (e.g. 99.99%) KE product is recovered through the solvent phase in the top liquid stream while greater than 90% of the water is removed through the water phase in the bottom liquid stream. Also, small amounts of solvent is lost through the water phase due to the miscibility of the solvent in the water phase. Column C1 is the solvent recovery column. Ketone Ester and some impurities are recovered from the bottom while water and solvent are recovered through the overhead and recycled back to the contact column. A small makeup stream is required to compensate for the solvent loss in contact and solvent recovery columns. Due to the large flow of the water, contact and solvent recovery columns are relatively large diameter columns. Columns C1 and C2 are utilized to remove the heavy boilers and light boilers and therefore, polish the final product.

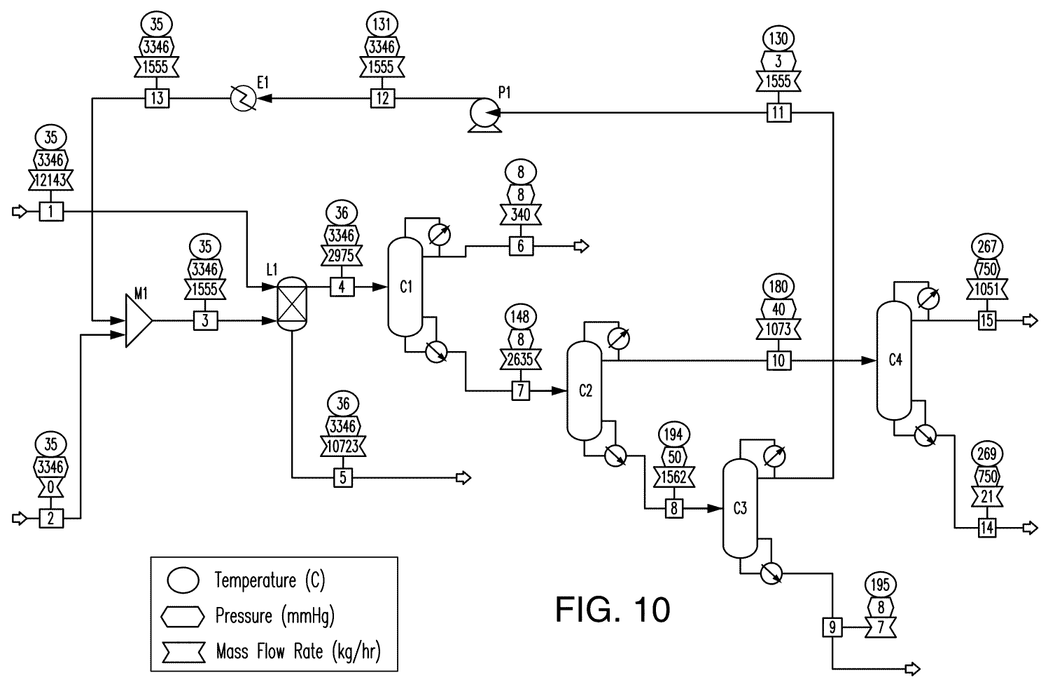

FIG. 10 shows a similar process when a higher boiling point solvent (i.e. TBP) is used. In this process since the boiling point of the solvent is higher, light ends removal and product recovery should be carried out before solvent is recovered. In this process light end materials are removed by column C1, and column C2 is used to separate the KE product from the solvent and other heavy boilers. Solvent is recovered through the overhead of the solvent recovery column C3 and is recycled back to the solvent contact column. Column C4 is an optional polishing column to further purify the KE product.

Throughout this application various publications have been referenced within parentheses. The disclosures of these publications in their entireties are hereby incorporated by reference in this application in order to more fully describe the state of the art to which this disclosure pertains.

Although the disclosure has been described with reference to the disclosed embodiments, those skilled in the art will readily appreciate that the specific examples and studies detailed above are only illustrative of the disclosure. It should be understood that various modifications can be made without departing from the spirit of the disclosure. Accordingly, the disclosure is limited only by the following claims.

What is claimed is:

1. A process for preparing (3R)-hydroxybutyl (3R)-hydroxybutyrate, comprising the steps of:
(a) performing a first esterification between a C₁-C₃ alcohol and (R)-3-hydroxybutyric acid to form a first esterification product and water in a first esterification product stream
(b) subjecting the first esterification product stream to distillation to remove water to form a concentrated first esterification product stream;
(c) subjecting the concentrated first esterification product stream to distillation to form an enriched first esterification product stream and a heavies stream comprising (R)-3-hydroxybutyric acid
(d) subjecting the enriched first esterification product stream to a second esterification with (R)-1,3-butanediol to produce (3R)-hydroxybutyl (3R)-hydroxybutyrate and the C₁-C₃ alcohol in a second esterification product stream.

2. The process of claim 1, wherein the heavies stream comprising C₁-C₃ alcohol is recycled into the first esterification.

3. The process of claim 1 or claim 2, further comprising subjecting the second esterification product stream to a purification procedure.

4. The process of claim 3, wherein the purification procedure comprises distillation.

5. The process of claim 4, wherein distillation comprises:
(a) subjecting the second esterification product stream to a first column distillation procedure to remove materials with a boiling point lower than (3R)-hydroxybutyl (3R)-hydroxybutyrate from the second esterification product stream to produce a first (3R)-hydroxybutyl (3R)-hydroxybutyrate-containing product stream; and
(b) subjecting the first (3R)-hydroxybutyl (3R)-hydroxybutyrate-containing product stream to a second column distillation procedure to remove materials with boiling points higher than (3R)-hydroxybutyl (3R)-hydroxybutyrate as a first high-boilers stream, to produce a purified (3R)-hydroxybutyl (3R)-hydroxybutyrate product.

6. The process of claim 5, further comprising:
(c) subjecting the first high-boilers stream to wiped-film evaporation (WFE) to produce a first WFE distillate and subjecting the first WFE distillate to step (b).

7. The process of claim 5 or claim 6, further comprising:
(d) subjecting the first (3R)-hydroxybutyl (3R)-hydroxybutyrate-containing product stream, prior to performing step (b), to an intermediate column distillation procedure to remove materials with boiling points higher than (3R)-hydroxybutyl (3R)-hydroxybutyrate as a second high-boilers stream; and
(e) subjecting the second high-boilers stream to wiped-film evaporation (WFE) producing a second WFE distillate and subjecting the second WFE distillate to step (d).

8. The process of any one of claims 1-7, wherein the C₁-C₃ alcohol generated during the second esterification is recovered and recycled.

9. The process of any one of claims 1-8, wherein the C₁-C₃ alcohol generated during the second esterification is aqueous.

10. The process of any one of claims 1-9, wherein the first column distillation procedure and second column distillation procedures are each performed at pressures equal to or less than atmospheric pressure.

11. The process of any one of claims 1-10, wherein the pressure of the first column distillation procedure differs from the pressure of the second distillation procedure.

12. The process of any one of claims 1-11, wherein the process further comprises subjecting the purified (3R)-hydroxybutyl (3R)-hydroxybutyrate product to a polishing column.

13. The process of claim 12, wherein the polishing column is an ion exchange column.

14. The process of claim 13, wherein the ion exchange column uses an exchange resin that is an anion exchange resin.

15. The process of claim 13, wherein the ion exchange column uses an exchange resin that is a cation exchange resin.

16. The process of any one of claims 1-15, wherein the purified (3R)-hydroxybutyl (3R)-hydroxybutyrate product is at least 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% pure.

17. The process of any one of claims 1-16, wherein the first esterification is promoted with an acid.

18. The process of claim 17, wherein the acid is selected from sulfuric acid, hydrochloric acid, acetic acid, benzoic acid, tosylic acid, candium(III) triflate, trifluoroacetic acid, phosphoric acid nitric acid, sulfamic acid, sulfonic acids, formic acid, acetic acid, lactic acid, propionic acid, oxalic acid, malonic acid, succinic acid, glutaric acid, or adipic acid.

19. The process of any one of claims 1-18, wherein the first esterification is promoted with an immobilized enzyme.

20. The process of claim 19, wherein the immobilized enzyme is a lipase.

21. The process of claim 20, wherein the lipase is selected from Novozyme 435, patatin, or Candida.

22. The process of claim 19, wherein the immobilized enzyme is an esterase.

23. The process of claim 22, wherein the esterase is a carboxylesterase.

24. The process of any one of claims 1-23, wherein the second esterification is promoted with an immobilized enzyme.

25. The process of claim 24, wherein the immobilized enzyme is a lipase.

26. The process of claim 25, wherein the lipase is selected from Novozyme 435, patatin, or Candida.

27. The process of claim 24, wherein the immobilized enzyme is an esterase.

28. The process of claim 27, wherein the esterase is a carboxylesterase.

29. The process of any one of claims 1-28, wherein the second esterification is promoted with an acid.

30. The process of claim 29, wherein the acid is selected from sulfuric acid, hydrochloric acid, acetic acid, benzoic acid, tosylic acid, candium(III) triflate, trifluoroacetic acid, phosphoric acid nitric acid, sulfamic acid, sulfonic acids, formic acid, acetic acid, lactic acid, propionic acid, oxalic acid, malonic acid, succinic acid, glutaric acid, or adipic acid.

31. The process of any one of claims 1-30, wherein water is removed during the esterification reaction.

32. The process of claim 31, wherein the water removal during the esterification reaction is accomplished with reactive distillation.

33. A process for preparing (3R)-hydroxybutyl (3R)-hydroxybutyrate, the process comprising:
(a) isolating (R)-3-hydroxybutyric acid from a fermentation broth;
(b) reacting (R)-3-hydroxybutyric acid with a C₁-C₃ alcohol to form a first esterification product stream;
(c) isolating (R)-1,3-butanediol from a fermentation broth to form a (R)-1,3-butanediol containing stream;
(d) combining the first esterification product stream with the (R)-1,3-butanediol containing stream in the presence of an esterifying agent to produce a (3R)-hydroxybutyl (3R)-hydroxybutyrate product stream; and
(e) purifying the (3R)-hydroxybutyl (3R)-hydroxybutyrate product stream.

34. The process of claim 33, wherein the (R)-3-hydroxybutyric acid from a fermentation broth is made by culturing a non-naturally occurring microbial organism.

35. The process of claim 34, wherein the non-naturally occurring microbial organism comprises a (3R)-hydroxybutyrate pathway.

36. The process of claim 35, wherein the (3R)-hydroxybutyrate pathway comprises a pathway selected from:
(1) 2B, 2C, and 2I;
(2) 2B, and 2H;
(3) 2J, 2K, 2C, and 2I;
(4) 2J, 2K, and 2H;
(5) 2A, 2B, 2C, and 2I;
(6) 2A, 2B, and 2H;
(7) 2A, 2J, 2K, 2C, and 2I;
(8) 2A, 2J, 2K, and 2H;
(9) 2E, 2F, 2B, 2C, and 2I;
(10) 2E, 2F, 2B, and 2H;
(11) 2E, 2F, 2J, 2K, 2C, and 2I;
(12) 2E, 2F, 2J, 2K, and 2H;
(13) 3A, 3B, and 3G;
(14) 3A, 3C, 2B, and 2H;
(15) 3A, 3C, 2B, 2C, and 2I;
(16) 3A, 3C, 2J, 2K, and 2H; and
(17) 3A, 3C, 2J, 2K, 2C, and 2I,
wherein 2A is an acetoacetyl-CoA thiolase, wherein 2B is a (3R)-hydroxybutyryl-CoA dehydrogenase, wherein 2C is a (3R)-hydroxybutyryl-CoA reductase, wherein 2E is an acetyl-CoA carboxylase, wherein 2F is an acetoacetyl-CoA synthase, wherein 2G is an acetoacetyl-CoA transferase, an acetoacetyl-CoA synthetase or an acetoacetyl-CoA hydrolase, wherein 2H is a (3R)-hydroxybutyryl-CoA transferase, a (3R)-hydroxybutyryl-CoA synthetase, or a (3R)-hydroxybutyryl-CoA hydrolase, wherein 2I is a (3R)-hydroxybutyraldehyde dehydrogenase, a (3R)-hydroxybutyraldehyde oxidase or a (3R)-hydroxybutyrate reductase, wherein 2J is a (3S)-hydroxybutyryl-CoA dehydrogenase, wherein 2K is a 3-hydroxybutyryl-CoA epimerase, wherein 3A is a 3-ketoacyl-ACP synthase, wherein 3B is an acetoacetyl-ACP reductase, wherein 3C is an acetoacetyl-CoA:ACP transferase, wherein 3G is an (3R)-hydroxybutyryl-ACP thioesterase.

37. The process of any one of claims 33-36, wherein the (R)-1,3-butanediol from a fermentation broth is made by culturing a non-naturally occurring microbial organism.

38. The process of one of claims 33-37, wherein the non-naturally occurring microbial organism comprises a (R)-1,3-butanediol pathway.

39. The process of claim 38, wherein the (R)-1,3-butanediol pathway comprises a pathway selected from:
(1) 2B, 2C, and 2D;
(2) 2B, 2H, 2I,and 2D;
(3) 2J, 2K, 2C, and 2D;
(4) 2J, 2K, 2H, 2I,and 2D;
(5) 2A, 2B, 2C, and 2D;
(6) 2A, 2B, 2H, 2I,and 2D;
(7) 2A, 2J, 2K, 2C, and 2D;
(8) 2A, 2J, 2K, 2H, 2I,and 2D;
(9) 2E, 2F, 2B, 2C, and 2D;
(10) 2E, 2F, 2B, 2H, 2I,and 2D;
(11) 2E, 2F, 2J, 2K, 2C, and 2D;
(12) 2E, 2F, 2J, 2K, 2H, 2I,and 2D;
(13) 3A, 3B, and 3E;
(14) 3A, 3C, 2B, 2C, and 2D;
(15) 3A, 3C, 2B, 2H, 2I,and 2D;
(16) 3A, 3C, 2J, 2K, 2C, and 2D;
(17) 3A, 3C, 2J, 2K, 2H, 2I,and 2D;
(18) 3A, 3B, 3D, 2C, and 2D;
(19) 3A, 3B, 3D, 2H, 2I,and 2D;
(20) 3A, 3B, 3G, 2I,and 2D; and
(21) 3A, 3B, 3F, and 2D,
wherein 2A is an acetoacetyl-CoA thiolase, wherein 2B is a (3R)-hydroxybutyryl-CoA dehydrogenase, wherein 2C is a (3R)-hydroxybutyryl-CoA reductase, wherein 2D is a (3R)-hydroxybutyraldehyde reductase, wherein 2E is an acetyl-CoA carboxylase, wherein 2F is an acetoacetyl-CoA synthase, wherein 2H is a (3R)-hydroxybutyryl-CoA transferase, a (3R)-hydroxybutyryl-CoA synthetase, or a (3R)-hydroxybutyryl-CoA hydrolase, wherein 2I is a (3R)-hydroxybutyraldehyde dehydrogenase, (3R)-hydroxybutyraldehyde oxidase or (3R)-hydroxybutyrate reductase, wherein 2J is a (3S)-hydroxybutyryl-CoA dehydrogenase, wherein 2K is a 3-hydroxybutyryl-CoA epimerase, wherein 3A is a 3-ketoacyl-ACP synthase, wherein 3B is an acetoacetyl-ACP reductase, wherein 3C is an acetoacetyl-CoA:ACP transferase, wherein 3D is a (3R)-hydroxybutyryl-CoA:ACP transferase, wherein 3E is a (3R)-hydroxybutyryl-ACP reductase (alcohol forming), wherein 3F is a (3R)-hydroxybutyryl-ACP reductase (aldehyde forming), wherein 3G is a (3R)-hydroxybutyryl-ACP thioesterase.

40. The process of any one of claims 33-39, wherein (R)-3-hydroxybutyric acid is produced from glucose, xylose, arabinose, galactose, mannose, fructose, sucrose or starch according to a fermentation process.

41. The process of any one of claims 33-40, wherein (R)-1,3-butanediol is produced from glucose, xylose, arabinose, galactose, mannose, fructose, sucrose or starch according to a fermentation process.

42. The process of any one of claims 33-41, wherein the esterifying agent is an acid.

43. The process of claim 42, wherein the acid is selected from sulfuric acid, hydrochloric acid, acetic acid, benzoic acid, tosylic acid, candium(III) triflate, trifluoroacetic acid, phosphoric acid nitric acid, sulfamic acid, sulfonic acids, formic acid, acetic acid, lactic acid, propionic acid, oxalic acid, malonic acid, succinic acid, glutaric acid, or adipic acid.

44. The process of any one of claims 33-43, wherein the esterifying agent is an immobilized enzyme.

45. The process of claim 44, wherein the immobilized enzyme is a lipase.

46. The process of claim 45, wherein the lipase is Novozyme 435, patatin, or Candida.

47. The process of claim 44, wherein the immobilized enzyme is an esterase.

48. The process of claim 47, wherein the esterase is a carboxylesterase.

49. The process of any one of claims 33-48, wherein the purification comprises a liquid-liquid extraction, distillation, filtration, or a combination thereof.

50. The process of claim 49, wherein the filtration is a microfiltration, nanofiltration, an ultrafiltration, or a combination thereof.

51. The process of claim 49, wherein the distillation comprises:
(a) subjecting the (3R)-hydroxybutyl (3R)-hydroxybutyrate product stream to a first column distillation procedure to remove materials with a boiling point lower than (3R)-hydroxybutyl (3R)-hydroxybutyrate from the (3R)-hydroxybutyl (3R)-hydroxybutyrate product stream to produce a first (3R)-hydroxybutyl (3R)-hydroxybutyrate-containing product stream; and
(b) subjecting the first (3R)-hydroxybutyl (3R)-hydroxybutyrate-containing product stream to a second column distillation procedure to remove materials with boiling points higher than (3R)-hydroxybutyl (3R)-hydroxybutyrate as a first high-boilers stream, to produce a purified (3R)-hydroxybutyl (3R)-hydroxybutyrate product.

52. The process of claim 51, further comprising:
(c) subjecting the first high-boilers stream to wiped-film evaporation (WFE) to produce a first WFE distillate and subjecting the first WFE distillate to step (b).

53. The process of claim 51 or claim 52, further comprising:
(d) subjecting the first (3R)-hydroxybutyl (3R)-hydroxybutyrate-containing product stream, prior to performing step (b), to an intermediate column distillation procedure to remove materials with boiling points higher than (3R)-hydroxybutyl (3R)-hydroxybutyrate as a second high-boilers stream; and
(e) subjecting the second high-boilers stream to wiped-film evaporation (WFE) producing a second WFE distillate and subjecting the second WFE distillate to step (d).

54. The process of any one of claims 51-53, wherein the first column distillation procedure and second column distillation procedures are each performed at pressures equal to or less than atmospheric pressure.

55. The process of any one of claims 51-54, wherein the pressure of the first column distillation procedure differs from the pressure of the second distillation procedure.

56. The process of any one of claims 51-55, wherein the process further comprises subjecting the purified (3R)-hydroxybutyl (3R)-hydroxybutyrate product to a polishing column.

57. The process of claim 56, wherein the polishing column is an ion exchange column.

58. The process of claim 57, wherein the ion exchange column uses an exchange resin that is an anion exchange resin.

59. The process of claim 57, wherein the ion exchange column uses an exchange resin that is a cation exchange resin.

60. The process of any one of claims 51-59, wherein the purified (3R)-hydroxybutyl (3R)-hydroxybutyrate is at least 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% pure.

61. The process of any one of claims 33-49, wherein step (d) is accomplished with reactive distillation.

62. The process of any one of claims 33-49 or 61, wherein the C₁-C₃ alcohol generated in the (3R)-hydroxybutyl (3R)-hydroxybutyrate product stream is recovered and recycled.

63. The process of claims 62, wherein the C₁-C₃ alcohol generated in the (3R)-hydroxybutyl (3R)-hydroxybutyrate product stream is aqueous.

64. The process of any one of claims 33-49, wherein isolating (R)-3-hydroxybutyric acid from a fermentation broth comprises:
separating a liquid fraction enriched in (R)-3-hydroxybutyric acid from a solid fraction comprising cells, wherein said step of separating said liquid fraction comprises one or more processes selected from the group consisting of microfiltration, ultrafiltration and nanofiltration;
removing salts from said liquid fraction, wherein salts are removed by ion exchange;
reducing water from said liquid fraction, wherein removing water is accomplished by evaporation; and
purifying (R)-3-hydroxybutyric acid from said liquid fraction.

65. The process of any one of claims 33-49, wherein isolating (R)-1,3-butanediol from a fermentation broth comprises
separating a liquid fraction enriched in (R)-1,3-butanediol from a solid fraction comprising cells, wherein said step of separating said liquid fraction comprises one or more processes selected from the group consisting of microfiltration, ultrafiltration and nanofiltration;
removing salts from said liquid fraction, wherein salts are removed by ion exchange;
reducing water from said liquid fraction, wherein removing water is accomplished by evaporation; and
purifying (R)-1,3-butanediol from said liquid fraction.

66. The process of any one of claims 33-49, wherein purifying (3R)-hydroxybutyl (3R)-hydroxybutyrate comprises:
contacting the (3R)-hydroxybutyl (3R)-hydroxybutyrate product stream with an extraction solvent in a solvent contact column to make an extraction solvent enriched in (3R)-hydroxybutyl (3R)-hydroxybutyrate;
removing the extraction solvent enriched in (3R)-hydroxybutyl (3R)-hydroxybutyrate; and
subjecting the extraction solvent enriched in (3R)-hydroxybutyl (3R)-hydroxybutyrate to a purification process.

67. The process of claim 66, wherein the extraction solvent has a boiling point lower than (3R)-hydroxybutyl (3R)-hydroxybutyrate.

68. The process of claim 66 or claim 67, wherein the extraction solvent is 1-hexanol or 1-butanol.

69. The process of any one of claims 66-68, wherein the purification process comprises distillation.

70. The process of claim 69, wherein distillation comprises:
(a) subjecting the extraction solvent enriched in (3R)-hydroxybutyl (3R)-hydroxybutyrate to a first column distillation procedure to remove materials with a boiling point lower than (3R)-hydroxybutyl (3R)-hydroxybutyrate from the extraction solvent enriched in (3R)-hydroxybutyl (3R)-hydroxybutyrate to produce a first (3R)-hydroxybutyl (3R)-hydroxybutyrate-containing product stream and a recovered extraction solvent stream; and
(b) subjecting the first (3R)-hydroxybutyl (3R)-hydroxybutyrate-containing product stream to a second column distillation procedure to remove materials with boiling points higher than (3R)-hydroxybutyl (3R)-hydroxybutyrate as a first high-boilers stream, to produce a purified (3R)-hydroxybutyl (3R)-hydroxybutyrate product.

71. The process of claim 70, further comprising:
(c) subjecting the first high-boilers stream to wiped-film evaporation (WFE) to produce a first WFE distillate and subjecting the first WFE distillate to step (b).

72. The process of claim 70 or claim 71, further comprising:
(d) subjecting the first (3R)-hydroxybutyl (3R)-hydroxybutyrate-containing product stream, prior to performing step (b), to an intermediate column distillation procedure to remove materials with boiling points higher than (3R)-hydroxybutyl (3R)-hydroxybutyrate as a second high-boilers stream; and
(e) subjecting the second high-boilers stream to wiped-film evaporation (WFE) producing a second WFE distillate and subjecting the second WFE distillate to step (d).

73. The process of any one of claims 70-72, wherein the (3R)-hydroxybutyl (3R)-hydroxybutyrate is bioderived.

74. The process of any one of claims 70-73, wherein the first column distillation procedure and second column distillation procedures are each performed at pressures equal to or less than atmospheric pressure.

75. The process of any one of claims 70-74, wherein the pressure of the first column distillation procedure differs from the pressure of the second distillation procedure.

76. The process of any one of claims 70-75, wherein the process further comprises subjecting the purified (3R)-hydroxybutyl (3R)-hydroxybutyrate product to a polishing column.

77. The process of claim 76, wherein the polishing column is an ion exchange column.

78. The process of claim 77, wherein the ion exchange column uses an exchange resin that is an anion exchange resin.

79. The process of claim 77, wherein the ion exchange column uses an exchange resin that is a cation exchange resin.

80. The process of any one of claims 70-79, wherein the recovered extraction solvent stream is recycled to the solvent contact column.

81. The process of any one of claims 70-80, wherein the purified (3R)-hydroxybutyl (3R)-hydroxybutyrate product is greater than 90% (w/w), 91% (w/w), 92% (w/w), 93% (w/w), 94% (w/w), 95% (w/w), 96% (w/w), 97% (w/w), 98% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w) or 99.9% (w/w), (3R)-hydroxybutyl (3R)-hydroxybutyrate.

82. The process of any one of claims 70-81, wherein recovery of (3R)-hydroxybutyl (3R)-hydroxybutyrate in the purified (3R)-hydroxybutyl (3R)-hydroxybutyrate product from the crude (3R)-hydroxybutyl (3R)-hydroxybutyrate mixture is greater than 40%, 50%, 60%, 70%, 80%, 90%, 95%, 96%, 97%, 98% or 99%.

83. The process of any one of claims 70-82, wherein the purified (3R)-hydroxybutyl (3R)-hydroxybutyrate is enantiopure.

84. The process of any one of claims 66-68, wherein the diameter of the solvent contact column is 1 cm to 10 m.

85. The process of any one of claims 66-68 or 84, wherein the solvent contact column is static.

86. The process of claim 85, wherein the static solvent contact column is a structured packing column, random packing column, or a column comprising a sieve tray.

87. The process of any one of claims 66-68 or 84, wherein the solvent contact column is agitated.

88. The process of claim 87, wherein the solvent contact column is agitated for a period of time.

89. The process of claim 88, wherein the agitation period is 1 second to 10 hours.

90. The process of claim 87, wherein the agitated solvent contact column is a rotating disc contactor or a pulsed column.

91. The process of claim 87, wherein the agitated solvent contact column is a Karr® column.

92. The process of claim 87, wherein the agitated solvent contact column is a Scheibel® column.

93. The process of any one of claims 66-68 or 84, wherein the solvent contact column is a mixer-settler.

94. The process of any one of claims 66-68 or 84-93, wherein the extraction solvent has a boiling point higher than (3R)-hydroxybutyl (3R)-hydroxybutyrate.

95. The process of claim 94, wherein the extraction solvent is tributyl phosphate.

96. The process of claim 94 or claim 95, wherein the purification process comprises distillation.

97. The process of claim 96, wherein distillation comprises:
(a) subjecting the extraction solvent enriched in (3R)-hydroxybutyl (3R)-hydroxybutyrate to a first column distillation procedure to remove materials with a boiling point lower than (3R)-hydroxybutyl (3R)-hydroxybutyrate from the extraction solvent enriched in (3R)-hydroxybutyl (3R)-hydroxybutyrate to produce a first (3R)-hydroxybutyl (3R)-hydroxybutyrate-containing product stream; and
(b) subjecting the first (3R)-hydroxybutyl (3R)-hydroxybutyrate-containing product stream to a second column distillation procedure to remove materials with boiling points higher than (3R)-hydroxybutyl (3R)-hydroxybutyrate as a first high-boilers stream, to produce a purified (3R)-hydroxybutyl (3R)-hydroxybutyrate product and a recovered extraction solvent stream.

98. The process of claim 97, further comprising:
(c) subjecting the first high-boilers stream to wiped-film evaporation (WFE) to produce a first WFE distillate and subjecting the first WFE distillate to step (b).

99. The process of claim 97 or claim 98, further comprising:
(d) subjecting the first (3R)-hydroxybutyl (3R)-hydroxybutyrate-containing product stream, prior to performing step (b), to an intermediate column distillation procedure to remove materials with boiling points higher than (3R)-hydroxybutyl (3R)-hydroxybutyrate as a second high-boilers stream; and
(e) subjecting the second high-boilers stream to wiped-film evaporation (WFE) producing a second WFE distillate and subjecting the second WFE distillate to step (d).

100. The process of any one of claims 97-99, wherein the first column distillation procedure and second column distillation procedures are each performed at pressures equal to or less than atmospheric pressure.

101. The process of any one of claims 97-100, wherein the pressure of the first column distillation procedure differs from the pressure of the second distillation procedure.

102. The process of any one of claims 97-101, wherein the recovered extraction solvent stream is recycled to the solvent contact column.

103. The process of any one of claims 97-102, wherein the process further comprises subjecting the purified (3R)-hydroxybutyl (3R)-hydroxybutyrate product to a polishing column.

104. The process of claim 103, wherein the polishing column is an ion exchange column.

105. The process of claim 104, wherein the ion exchange column uses an exchange resin that is an anion exchange resin.

106. The process of claim 104, wherein the ion exchange column uses an exchange resin that is a cation exchange resin.

107. A process for preparing (3R)-hydroxybutyl (3R)-hydroxybutyrate, comprising the steps of
(a) performing an esterification reaction between ethyl (R)-3-hydroxybutanoate and (R)-1,3-butanediol in a reactor to form a product stream comprising (3R)-hydroxybutyl (3R)-hydroxybutyrate and ethanol;
(b) subjecting the product stream comprising (3R)-hydroxybutyl (3R)-hydroxybutyrate and ethanol to a first column distillation procedure to remove materials with a boiling point lower than (3R)-hydroxybutyl (3R)-hydroxybutyrate from the product stream to produce a first (3R)-hydroxybutyl (3R)-hydroxybutyrate-containing product stream;
(c) subjecting the first (3R)-hydroxybutyl (3R)-hydroxybutyrate-containing product stream to a second column distillation procedure to remove materials with boiling points higher than (3R)-hydroxybutyl (3R)-hydroxybutyrate as a first high-boilers stream, to produce a purified (3R)-hydroxybutyl (3R)-hydroxybutyrate product; and
(d) subjecting the first high-boilers stream to wiped-film evaporation (WFE) to produce a first WFE distillate and subjecting the first WFE distillate to step (c).

108. The process of claim 107, wherein the esterification reaction is promoted with an acid.

109. The process of claim 108, wherein the acid is selected from sulfuric acid, hydrochloric acid, acetic acid, benzoic acid, tosylic acid, candium(III) triflate, trifluoroacetic acid, phosphoric acid nitric acid, sulfamic acid, sulfonic acids, formic acid, acetic acid, lactic acid, propionic acid, oxalic acid, malonic acid, succinic acid, glutaric acid, or adipic acid.

110. The process of any one of claims 107-109, wherein the second esterification is promoted with an immobilized enzyme.

111. The process of claim 110, wherein the immobilized enzyme is a lipase.

112. The process of claim 111, wherein the lipase is selected from Novozyme 435, patatin, or Candida.

113. The process of claim 110, wherein the immobilized enzyme is an esterase.

114. The process of claim 113, wherein the esterase is a carboxylesterase.

115. The process of any one of claims 107-114, wherein the ethanol generated during the second esterification is recovered and recycled.

116. The process of claim 115, wherein the ethanol generated during the second esterification is aqueous.

117. The process of any one of claims 107-116, wherein the reactor operates at a temperature of 0° C. to 120° C.

118. The process of any one of claims 107-117, wherein the reactor operates at a temperature of 10° C. to 50° C.

119. The process of any one of claims 107-118, wherein the reactor operates under reduced pressure.

120. The process of claim 119, wherein the pressure is between 5 and 400 mmHg.

121. The process of any one of claims 107-120, wherein the reactor operates under positive pressure.

122. The process of claim 121, wherein the pressure is between 1 and 2 atmospheres.

123. The process of any one of claims 107-122, further comprising:
(e) subjecting the first (3R)-hydroxybutyl (3R)-hydroxybutyrate-containing product stream, prior to performing step (c), to an intermediate column distillation procedure to remove materials with boiling points higher than (3R)-hydroxybutyl (3R)-hydroxybutyrate as a second high-boilers stream; and
(f) subjecting the second high-boilers stream to wiped-film evaporation (WFE) producing a second WFE distillate and subjecting the second WFE distillate to step (e).

124. The process of any one of claims 107-123, wherein the purified (3R)-hydroxybutyl (3R)-hydroxybutyrate is at least 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% pure.

125. The process of any one of claims 107-124, wherein the first column distillation procedure and second column distillation procedures are each performed at pressures equal to or less than atmospheric pressure.

126. The process of any one of claims 107-125, wherein the pressure of the first column distillation procedure differs from the pressure of the second distillation procedure.

127. The process of any one of claims 107-126, wherein the process further comprises subjecting the purified (3R)-hydroxybutyl (3R)-hydroxybutyrate product to a polishing column.

128. The process of claim 127, wherein the polishing column is an ion exchange column.

129. The process of claim 128, wherein the ion exchange column uses an exchange resin that is an anion exchange resin.

130. The process of claim 128, wherein the ion exchange column uses an exchange resin that is a cation exchange resin.

131. The process of any one of claims 107-130, wherein the materials with a boiling point lower than (3R)-hydroxybutyl (3R)-hydroxybutyrate include (R)-3-hydroxybutanoate and (R)-1,3-butanediol.

132. The process of claim 131, wherein the (R)-3-hydroxybutanoate and (R)-1,3-butanediol are recycled back into the reactor.

133. The process of any one of claims 107-132, wherein the ethanol is removed during the esterification reaction.

134. The process of any one of claims 107-133, wherein ethanol removal during the esterification reaction is accomplished with reactive distillation.

135. A process for preparing (3R)-hydroxybutyl (3R)-hydroxybutyrate, comprising the steps of
(a) isolating (R)-3-hydroxybutanoic acid from a fermentation broth;
(b) performing an esterification reaction between (R)-3-hydroxybutanoic acid and (R)-1,3-butanediol in a reactor to form a product stream comprising (3R)-hydroxybutyl (3R)-hydroxybutyrate; and
(c) subjecting the product stream comprising (3R)-hydroxybutyl (3R)-hydroxybutyrate to a first column distillation procedure to remove materials with a boiling point lower than (3R)-hydroxybutyl (3R)-hydroxybutyrate from the product stream to produce a first (3R)-hydroxybutyl (3R)-hydroxybutyrate-containing product stream;
(d) subjecting the first (3R)-hydroxybutyl (3R)-hydroxybutyrate-containing product stream to a second column distillation procedure to remove materials with boiling points higher than (3R)-hydroxybutyl (3R)-hydroxybutyrate as a first high-boilers stream, to produce a purified (3R)-hydroxybutyl (3R)-hydroxybutyrate product; and
(e) subjecting the first high-boilers stream to wiped-film evaporation (WFE) to produce a first WFE distillate and subjecting the first WFE distillate to step (d).

136. The process of claim 135, wherein the esterification reaction is promoted with an acid.

137. The process of claim 136, wherein the acid is selected from sulfuric acid, hydrochloric acid, acetic acid, benzoic acid, tosylic acid, candium(III) triflate, trifluoroacetic acid, phosphoric acid nitric acid, sulfamic acid, sulfonic acids, formic acid, acetic acid, lactic acid, propionic acid, oxalic acid, malonic acid, succinic acid, glutaric acid, or adipic acid.

138. The process of any one of claims 135-137, wherein the second esterification is promoted with an immobilized enzyme.

139. The process of claim 138, wherein the immobilized enzyme is a lipase.

140. The process of claim 139, wherein the lipase is selected from Novozyme 435, patatin, or Candida.

141. The process of claim 138, wherein the immobilized enzyme is an esterase.

142. The process of claim 141, wherein the esterase is a carboxylesterase.

143. The process of any one of claims 135-142, wherein the reactor operates at a temperature of 0° C. to 120° C.

144. The process of any one of claims 135-143, wherein the reactor operates at a temperature of 10° C. to 50° C.

145. The process of any one of claims 135-144, wherein the reactor operates under reduced pressure.

146. The process of claim 145, wherein the pressure is between 5 and 400 mmHg.

147. The process of any one of claims 135-146, wherein the reactor operates under positive pressure.

148. The process of claim 147, wherein the pressure is between 1 and 2 atmospheres.

149. The process of any one of claims 135-148, further comprising:
(f) subjecting the first (3R)-hydroxybutyl (3R)-hydroxybutyrate-containing product stream, prior to performing step (d), to an intermediate column distillation procedure to remove materials with boiling points higher than (3R)-hydroxybutyl (3R)-hydroxybutyrate as a second high-boilers stream; and
(g) subjecting the second high-boilers stream to wiped-film evaporation (WFE) producing a second WFE distillate and subjecting the second WFE distillate to step (f).

150. The process of any one of claims 135-149, wherein the purified (3R)-hydroxybutyl (3R)-hydroxybutyrate is at least 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% pure.

151. The process of any one of claims 135-150, wherein the first column distillation procedure and second column distillation procedures are each performed at pressures equal to or less than atmospheric pressure.

152. The process of any one of claims 135-151, wherein the pressure of the first column distillation procedure differs from the pressure of the second distillation procedure.

153. The process of any one of claims 135-152, wherein the process further comprises subjecting the purified (3R)-hydroxybutyl (3R)-hydroxybutyrate product to a polishing column.

154. The process of claim 153, wherein the polishing column is an ion exchange column.

155. The process of claim 154, wherein the ion exchange column uses an exchange resin that is an anion exchange resin.

156. The process of claim 154, wherein the ion exchange column uses an exchange resin that is a cation exchange resin.

157. The process of any one of claims 135-156, wherein the materials with a boiling point lower than (3R)-hydroxybutyl (3R)-hydroxybutyrate include (R)-3-hydroxybutanoate and (R)-1,3-butanediol.

158. The process of claim 157, wherein the (R)-3-hydroxybutanoate and (R)-1,3-butanediol are recycled back into the reactor.

159. The process of any one of claims 135-158, wherein the water is removed during the esterification reaction.

160. The process of claim 159, wherein the water removal during the esterification reaction is accomplished with reactive distillation.

161. A process of isolating (3R)-hydroxybutyl (3R)-hydroxybutyrate from a fermentation broth comprising
(a) separating a liquid fraction enriched in (3R)-hydroxybutyl (3R)-hydroxybutyrate from a solid fraction comprising cells, wherein said step of separating said liquid fraction comprises one or more processes selected from the group consisting of microfiltration, ultrafiltration and nanofiltration;
(b) removing salts from said liquid fraction, wherein salts are removed by ion exchange;
(c) reducing water from said liquid fraction, wherein removing water is accomplished by evaporation, to form a concentrated liquid fraction;
(d) subjecting the concentrated liquid fraction to a first column distillation procedure to remove materials with boiling points higher than (3R)-hydroxybutyl (3R)-hydroxybutyrate from the concentrated liquid fraction containing (3R)-hydroxybutyl (3R)-hydroxybutyrate to produce a first (3R)-hydroxybutyl (3R)-hydroxybutyrate-containing product stream and a high-boilers stream;
(e) subjecting the first (3R)-hydroxybutyl (3R)-hydroxybutyrate-containing product stream to a second column distillation procedure to remove materials with boiling points lower than (3R)-hydroxybutyl (3R)-hydroxybutyrate as a first low-boilers stream, to produce a purified (3R)-hydroxybutyl (3R)-hydroxybutyrate product; and
(f) subjecting the high-boilers stream to wiped-film evaporation (WFE) to produce a first WFE distillate and subjecting the first WFE distillate to step (d).

162. The process of claim 161, wherein microfiltration comprises filtering through a membrane having a pore size from about 0.1 microns to about 5.0 microns.

163. The process of claim 161 or claim 162, wherein ultrafiltration comprises filtering through a membrane having a pore size from about 0.005 to about 0.1 microns.

164. The process of any one of claims 161-163, wherein nanofiltration comprises filtering through a membrane having a pore size from about 0.0005 microns to about 0.005 microns.

165. The process of any one of claims 161-164, wherein the evaporation is accomplished with an evaporator system.

166. The process of claim 165, wherein said evaporator system comprises an evaporator selected from the group consisting of a falling film evaporator, a short path falling film evaporator, a forced circulation evaporator, a plate evaporator, a circulation evaporator, a fluidized bed evaporator, a rising film evaporator, a counterflow-trickle evaporator, a stirrer evaporator, and a spiral tube evaporator.

167. The process of any one of claims 161-166, wherein the reduction of water is from about 85% by weight to about 15% by weight.

168. The process of any one of claims 161-167, wherein the (3R)-hydroxybutyl (3R)-hydroxybutyrate is bioderived.

169. The process of any one of claims 161-168, wherein the first column distillation procedure and second column distillation procedures are each performed at pressures equal to or less than atmospheric pressure.

170. The process of any one of claims 161-169, wherein the pressure of the first column distillation procedure differs from the pressure of the second distillation procedure.

171. The process of any one of claims 161-170, wherein the purified (3R)-hydroxybutyl (3R)-hydroxybutyrate product is greater than 90% (w/w), 91% (w/w), 92% (w/w), 93% (w/w), 94% (w/w), 95% (w/w), 96% (w/w), 97%, (w/w) 98% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w) or 99.9% (w/w), (3R)-hydroxybutyl (3R)-hydroxybutyrate.

172. The process of any one of claims 161-171, wherein recovery of (3R)-hydroxybutyl (3R)-hydroxybutyrate in the purified (3R)-hydroxybutyl (3R)-hydroxybutyrate product (3R)-hydroxybutyl (3R)-hydroxybutyrate is greater than 40%, 50%, 60%, 70%, 80%, 90%, 95%, 96%, 97%, 98% or 99%.

173. The process of any one of claims 161-172, wherein the fermentation broth comprises (3R)-hydroxybutyl (3R)-hydroxybutyrate at a concentration of about 5%-15% by weight of (3R)-hydroxybutyl (3R)-hydroxybutyrate.
